SEQUENCE LISTING (1 sequence)

SEQ ID NO: 1 - DNA, 60 bp, Homo sapiens
tggtggcggcggttggggctcggcgctcgctcgctcgctcgctgggcgggcggtgcgatg

(19) United States
(12) Patent Application Publication
(10) Pub. No.: US 2020/0390072 A1
(43) Pub. Date: Dec. 17, 2020

(54) IDENTIFYING AND CHARACTERIZING GENOMIC SAFE HARBORS (GSH) IN HUMANS AND MURINE GENOMES, AND VIRAL AND NON-VIRAL VECTOR COMPOSITIONS FOR TARGETED INTEGRATION AT AN IDENTIFIED GSH LOCI

(71) Applicants: Generation Bio Co., Cambridge, MA (US); University of Massachusetts, Boston, MA (US)

(72) Inventors: Robert M. Kotin, Cambridge, MA (US); Evin Hildebrandt, Boston, MA (US)

(21) Appl. No.: 16/977,517

(22) PCT Filed: Mar. 1, 2019
(86) PCT No.: PCT/US2019/020224

§ 371 Date: Sep. 2, 2020

Related U.S. Application Data
(60) Provisional application No. 62/743,811, filed on Oct. 10, 2018.
(60) Provisional application No. 62/716,421, filed on Aug. 9, 2018.
(60) Provisional application No. 62/637,583, filed on Mar. 2, 2018.

(51) Int. Cl. A01K 67/027; C07K 16/28; C12N 15/861; C12N 9/22; G16B 25/20

(52) U.S. Cl. CPC A01K 67/0278; C07K 16/2896; C12N 15/861; C12N 9/22; C12N 2506/02; A01K 2217/072; A01K 2227/105; A01K 2267/03; G16B 25/20

(57) ABSTRACT

The technology described herein relates to methods, compositions and in silico screening approaches for identifying and validating genomic safe harbors (GSHs) in mammalian genomes, including human genomes. Another aspects relates to recombinant nucleic acid vectors, including non-viral and viral vectors comprising a portion of the GSH loci, or gRNA sequences specific to a GSH loci, and methods for use of the vectors for insertion of a gene of interest into a GSH loci.

### CROSS REFERENCE TO RELATED APPLICATIONS

This application claims benefit under 35 U.S.C. § 119(e) of U.S. Provisional Application Nos. 62/637,586, filed Mar. 3, 2018 and 62/716,421, filed on Aug. 9, 2018, and 62/743,811, filed on Oct. 10, 2018, the content of each of which is incorporated herein by reference in its entirety.

### TECHNICAL FIELD

The present disclosure relates to the field of gene therapy, including identification, characterizing and validating genomic safe harbor (GSH) loci in mammalian, including human genomes. The disclosure relates to a method to identify the GSH, methods to validate the GSH, and recombinant nucleic acid constructs comprising nucleic acids complementary to regions of the GSH that guides homologous recombination with regions of the GSH, as well as cells, kits and transgenic animals comprising recombinant nucleic acid constructs.

### BACKGROUND

The modification of the human genome by the stable insertion of functional transgenes and other genetic elements is of great value in biomedical research and medicine. Several diseases have now been successfully treated with gene therapy. Genetically modified human cells are also valuable for the study of gene function, and for tracking and lineage analyses using reporter systems. All these applications depend on the reliable function of the introduced genes in their new environments. However, randomly inserted genes are subject to position effects and silencing, making their expression unreliable and unpredictable. Centromeres and sub-telomeric regions are particularly prone to transgene silencing. Reciprocally, newly integrated genes may affect the surrounding endogenous genes and chromatin, potentially altering cell behavior or favoring cellular transformation. Despite the successes of therapeutic gene transfer, there have been several cases of malignant transformation associated with insertional activation of oncogenes following stem cell gene therapy, emphasizing the importance of where newly integrated DNA locates.

Despite this, the gene editing field has evolved from classical but inefficient homologous recombination, to more specific and efficient DNA nuclease mediated recombination using zinc finger nuclease and TALENS, to widely used CRISPR/Cas9 nuclease technology. Because of the robustness of the CRISPR/Cas9 methodologies, gene editing has become routine for non-specialized research groups. However, the insertion of foreign DNA into the genome of progenitor cells may adversely affect terminal differentiation into specific cell types. A genomic safe harbor (GSH) refers to a genetic locus that accommodates the insertion of exogenous DNA with either constitutive or conditional expression activity without significantly affecting the viability of somatic cells, progenitor cells, or germ line cells and ontogeny.

The availability of such GSH loci would be extremely useful to express reporter genes, suicide genes, selectable genes or therapeutic genes. Three intragenic sites have been proposed as GSHs (AAV51, CCRS and ROSA26 and albumin in murine cells) (see, e.g., U.S. Pat. Nos. 7,951,925; 8,771,985; 8,110,379; 7,951,925; U.S. Publication Nos. 20100218264; 20110265198; 20130137104; 20130122591; 20130177983; 20130177960; 20150056705 and 20150159172). However, these proposed GSHs are in relatively gene-rich regions and are near genes that have been implicated in cancer. Genes that are adjacent to AAV51 may be spared by some promoters, but safety validation in multiple tissues remains to be carried out. Also, the dispensability of the disrupted gene, especially after biallelic disruption, as is often the case with endonuclease-mediated targeting, remains to be investigated further.

Therefore, the identification of more sites would be highly valuable, especially at extragenic or intergenic regions. There is also a need to identify, qualify and validate candidate GSH loci for research and potential therapeutic applications, in particular, because transgene expression may vary by GSH loci, developmental stage, and tissue type. In addition, the targeted cell “potency” may be affected in a GSH-dependent manner, for example, hematopoietic stem cells (HSC) and embryonic stem cells (ESC). Therefore, identifying multiple GSH loci in the human and mouse genomes may provide a catalog of sites for different applications, including e.g., expression of a nucleic acid of interest, such as, e.g., therapeutic RNA, miRNAs, therapeutic proteins and nucleic acids, and suicide genes and the like.

### SUMMARY

The disclosure herein relates to screening assays, including in silico approaches to identify genomic safe harbor loci in mammalian genomes, including human genomes, as well as methodological principles for selecting and validating GSHs, including use of any of: bioinformatics, expression arrays and transcriptome analyses (e.g., RNAseq) to query nearby genes, in vitro expression assays of inserted genes into the GSH, in vitro-directed differentiation or in vivo reconstitution assays in vitro and in xenogeneic transplant models, transgenesis in syntenic regions and analyses of patient and non-human genomic databases from individuals harboring integrated provirus sequences.

The technology described herein relates to methods, compositions and in silico screening approaches for identifying and validating genomic safe harbors (GSHs). GSHs are intragenic, intergenic, or extragenic regions of the human and model species genomes that are able to accommodate the predictable expression of newly integrated DNA without significant adverse effects on the host cell or organism. While not being limited to theory, a useful safe harbor must permit sufficient transgene expression to yield desired levels of the vector-encoded protein or non-coding RNA. A GSH also should not predispose cells to malignant transformation nor significantly alter normal cellular functions. What distinguishes a GSH from a fortuitous good integration event is the predictability of outcome, which is based on prior knowledge and validation of the GSH.

The discovery and validation of GSHs in the human genome will ultimately benefit human cell engineering and especially stem cell and gene therapy, and validation of true GSHs is important enabling safe clinical development and advancement of technologies and tools for targeted integration at a GSH loci, including targeting the GSH with nucleases specific for the safe harbor genes such that the transgene construct is inserted for example, by either homology direct repair (HDR) or non-homologous end-joining (NHEJ)-driven processes, where such technologies have preceded the identification of appropriate target sites.

One aspect of the technology disclosed herein relates to the identification of genomic safe harbors based on provirus insertions in germlines of related species within a taxonomic rank. The inventors have discovered that evolutionary conserved heritable endogenous virus elements (EVEs) effectively denote genomic loci that are tolerant of insertions in the germline. Species within a taxonomic rank with an EVE sequence at the same genomic locus confirm infection of an individual animal that was the common ancestor to species that radiated into the individual, thus defining that lineage as an EVE-positive clade. The persistence of the EVE allele(s) through multiple epochs of the Cenozoic Era can be attributed to a single individual infected with the virus either a population bottleneck or that the EVE provided a positive selective advantage (or less likely resulted from a random integration event into a benign locus resulting in neutrality, i.e., neither acts positively nor negatively, thereby is neutral and provides no selection benefits either way. However, the probability of stabilizing an allele within population is influenced by (i) Fitness conferred and (ii) the effective population of the species, i.e., the population of breeding animals within the group.

Another aspect of the technology described herein relates to a method to identify genomic safe harbors using comparative genomic approaches. In particular, one embodiment relates to a method to identify a GSH in a mammalian genome comprising comparing interspecific introns of collinearly organized and/or synteny organized genes to identify an enlarged intron in one species relative to another species, where the enlarged intron identifies a potential genomic safe. In another embodiment, a method to identify a GSH in a mammalian genome comprises comparing the intergenic distance (or space) between selected genes or adjacent genes of collinearly organized or synteny organized genes in different species to identify large variations in the intergenic spaces between the two selected genes in different species, and where there is a large variation in the intergenic space, it identifies a potential genomic safe harbor.

The disclosure herein relates to methods to identify GSH loci in a mammalian genome, including a human genome, as well as methods to validate the GSH loci. Other aspects of the technology relate to modifying the identified GSH loci and generation of GSH intermediates, e.g., a GSH that has been modified to comprise a multiple cloning site (MCS), or the like for insertion of a transgene at the identified GSH loci. GSH intermediates also refer to cells with partial recombination (i.e., where the site is nicked and recombined partially with a transgene to be inserted).

In some embodiments, the disclosure also relates to nucleic acid vector compositions, e.g., viral and non-viral vectors comprising at least a portion or region of the GSH identified using the methods disclosed herein. The portion or region of the GSH that can be modified, e.g., insertion of a transgene or alternatively, introduction of a point mutation (e.g., insertion, deletion, any disruption of the gene), or a stop codon to disrupt or knock-out the gene function of a GSH gene identified herein, which is useful for example, to validate and/or characterize the identified GSH loci. In other embodiments, the portion or region of the GSH in the vector can be modified to comprise a guide RNA (gRNA) inserted, e.g., a guide RNA for a nuclease as disclosed herein. In some embodiments, the GSH vector can comprise a target site for a guide RNA (gRNA) as disclosed herein, or alternatively, a restriction cloning site for introduction of a nucleic acid of interest as disclosed herein.

In alternative embodiments, the disclosure herein also relates to nucleic acid vector compositions comprising at GSH 5′-homology arm, and a GSH 3′-homology arm flanking a nucleic acid comprising a restriction cloning site, where the vector can be used to integrate the flanked nucleic acid into the genome at a GSH by homologous recombination. In all aspects as disclosed herein, the nucleic acid vector compositions can be a plasmid, cosmid, or artificial chromosome (e.g., BAC), minicircle nucleic acid, or recombinant viral vector (e.g., rAd, rAAV, rHSV, BEV or variants thereof).

Other aspects of the invention relate to methods to integrate a nucleic acid of interest into a genome at a GSH identified herein using the methods and vector compositions as disclosed herein. Other aspects relate to a cell, or transgenic animal with a nucleic acid of interest integrated into the genome using the methods and vector compositions as disclosed herein.

Yet other aspects of the invention relate to applications of the sequences present at the identified GSH sites in construction of variant viral capsids. The EVEs and other identified sequences located at the GSH of the invention (the “GSH sequence” or “GSH nucleic acid”) may represent ancient AAV capsid sequences that are no longer present in modern-day dependoparvovirus capsids. Such sequences may have useful properties, for example enhancement of dependoparvovirus stability and/or activity when combined with modern-day dependoparvovirus capsid sequences. In one embodiment, a modified dependoparvovirus is provided wherein a GSH sequence of the invention is inserted into the surface-exposed region (e.g., a variable region) of the dependoparvovirus capsid. In one aspect, the variable region of the dependoparvovirus capsid is selected from the variable region of AAV I, II, III, IV, V, VI, VII, VIII, and IX. In another aspect, the GSH sequence is an EVE. In another embodiment, a modified dependoparvovirus is provided wherein a GSH sequence of the invention is used as a short linear sequence inserted into a tertiary structural element of the dependoparvovirus. In one aspect, the tertiary structural element is a 3-fold axis of symmetry. In another aspect, the GSH sequence is an EVE. In another embodiment, the invention provides a method of constructing a modified dependoparvovirus comprising a variant capsid wherein the capsid comprises a GSH sequence of the invention. In one aspect, the GSH sequence is comprised in the variable region of the dependoparvovirus capsid. In another aspect, the GSH sequence is comprised in a tertiary structural element of the dependoparvovirus. In another aspect, the GSH sequence is an EVE.

The methods and compositions described herein can be used in methods comprising homology recombination, for example, as described in Rouet et al. Proc Natl Acad Sci 91:6064-6068 (1994); Chu et al. Nat Biotechnol 33:543-548 (2015); Richardson et al. Nat Biotechnol 33:339-344 (2016); Komor et al. Nature 533:420-424 (2016); the contents of each of which are incorporated by reference herein in their entirety.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a schematic representation of the PAX5 gene located on Chromosome 9: 36,833,275-37,034,185 reverse strand (GRCh38: CM000671.2), and neighboring/surrounding genes or RNA sequences, such as those listed in Table 1A.

FIG. 2 shows Table 1A listing candidate GSH regions or genes identified using the methods disclosed herein.

FIG. 3 shows Table 1B listing of intergenic loci and intragenic loci candidate GSH regions or genes identified using the methods disclosed herein.

FIG. 4A shows Table 2 of Endogenous viral elements (EVE) related to single stranded DNA viruses (reproduced from Supplemental Table S6 from Katzourakis A, Gifford R J (2010) Endogenous Viral Elements in Animal Genomes. PLoS Genet 6(11): e1001191, which is incorporated herein in its entirety by reference). ¹ Common name of host species. Numbers in parentheses indicate the total number of matches identified where only a subset are shown. ²GenBank accession number of the contig containing the EVE sequence. ³Location of EVE sequence within contig. ⁴EVE orientation relative to contig. ⁵Accession number and ⁶e-value of best matching of best matching viral sequence, based on tBLASTn search against Genbank with putative EVE peptides (see methods section). ⁷e-value of putative EVE peptide sequence to top-scoring PFAM database viral match (a removed stop codons). ⁸Location of EVE nucleotide sequence relative to type species virus of the most closely related virus genus, based on pairwise tBLASTn with EVE peptide. ⁹Element names are shown for elements that were orthologous across one or more host taxa (see methods section). Names follow the convention of Horie et al for Bornavirus-related elements). Abbreviations: AAV=adeno-associated virus; MVM=minute virus of mice; AMDV=Aleutian mink disease virus; PCV-1=porcine circovirus type—

FIG. 4B shows Table 4A of the Dependovirus sequence information. Legend: Complete gene (F), Partial gene (P), * This dataset is from metagenomic study from Brazil.

FIG. 5 shows Table 3 listing exemplary genes for nucleic acid of interest.

FIG. 6 shows Table 6 listing exemplary genetic diseases for treatment using the vector compositions.

FIG. 7 provides an MDS plot comparing the transcriptional profiles of cells comprising GFP inserts in one of five loci: AAVs1, Kif6, Pax5, SRF, or DCTN, in comparison with wild-type cells, as described in Example 1.

FIG. 8 provides a graph showing the relative ratio of expression of GFP inserted at a target locus in HEK293 cells normalized to the expression of GAPDH in that cell, as described in Example 1.

### DETAILED DESCRIPTION

The technology described herein relates to methods, compositions, and in silco screening approaches for identifying, characterizing and validating genomic safe harbor (GSH) loci in mammalian, including human genomes. Embodiments of the invention also relate to method to identify the GSH, methods to validate the GSH, and recombinant nucleic acid constructs comprising nucleic acids complementary to regions of the GSH that guide homologous recombination with regions of the GSH, as well as modified AAV incorporating one or more GSH sequence in their capsid, and cells, kits and transgenic animals comprising recombinant nucleic acid constructs.

### I. Identifying Genomic Safe Harbors Using EVEs of Proto-Species or Related Species in a Taxonomic Order

### I. Identifying Genomic Safe Harbors Using EVEs of Proto-Species or Related Species in a Taxonomic Order

One aspect of the technology described herein provide methods to identify genomic safe harbors using evolutionary biology to identify AAV- and parvovirus or provirus remnants, referred to as endogenous virus elements (EVEs), in related species within a taxonomic rank. The results described herein demonstrate that EVEs can be acquired into the germline of a usually extinct ur-species prior to the radiation of the species, such that all evolved or descendent species retain the EVE allele. Whereas closely related species that evolved or radiated prior to the “endogenization” event retain empty loci. That is, the speciation occurred subsequent to EVE acquisition are therefore is monophyletic. As an illustrative example only, the locus occupied by intergenic EVE in the Macropodidae (kangaroos and related species) is identifiable in other marsupials, including Didelphis virgiana (North American opossum). These unoccupied loci are identifiable in other taxonomic families and although the EVE open reading frames are disrupted, the virus sequence represents foreign DNA inserted into the genome of the totipotent germ cell, thus identifying candidate genomic safe-harbor loci.

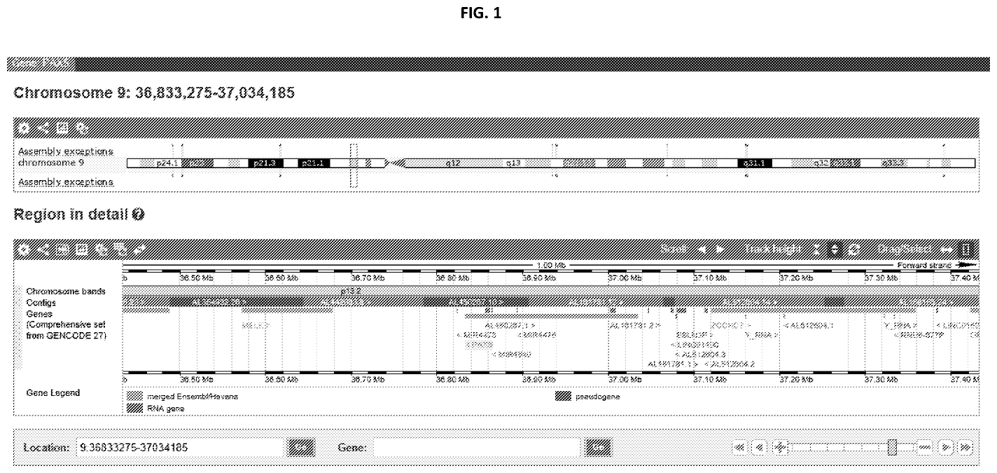

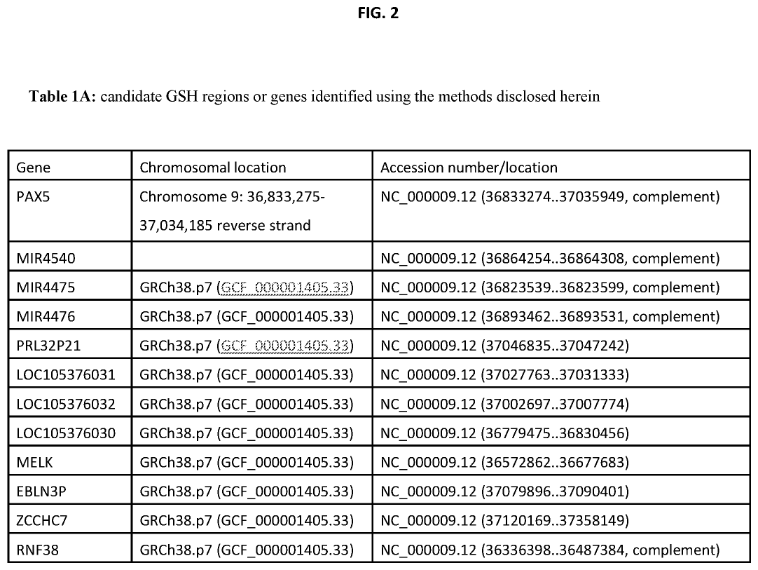

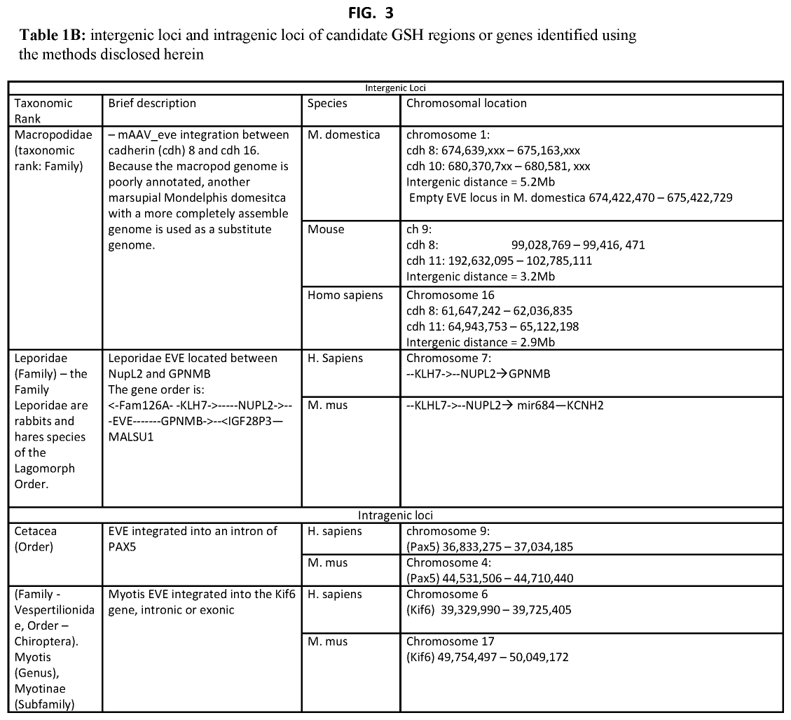

In some embodiments, the method utilizes interspecific synteny to identify orthologous safe-harbors in the murine and human genomes with potential usefulness in genome editing techniques, such as with mega-nucleases or CRISPR/Cas9 approaches. For example, all Cetacea have an intronic AAV EVE in the PAX5 gene. PAX5 gene (also known as “B-cell lineage specific activator” or BSAP). The homeodomain transcription factor, PAX5 is conserved in vertebrates, for example, human, chimp, macaque, mouse, rat, dog, horse, cow, pig, opossum, platypus, chicken, lizard, xenopus, c. elegans, drosphila and zebrafish. In humans, the PAX5 gene is located on human chromosome 9 at positions: 36,833,275-37,034,185 reverse strand (GRCh38: CM000671.2) or 36,833,272-37,034,182 in GRCh37 coordinates (see FIG. 1) also referred to as 9p13.2.

As an exemplary Example, the inventors assessed if this EVE locus, e.g., the PAX5 gene is a safe-harbor by inserting a reporter gene into the orthologous region in human progenitor cells. In some embodiments, mouse and human lymphomyeloid stem cells are used, which can be manipulated ex vivo and then engrafted into immune-cell depleted mice. The lymphomyeloid repopulate the lineages which are easily characterized with cell surface markers. Transgenic mice can also be used to test of the breadth of the safe-harbor into other tissues and systems.

In some embodiments, the method to identify a GSH in a mammalian genome comprises an initial sequencing and/or in silico analysis of the sequence of genomic DNA inferred from an ur-species by multiple species within a taxonomic rank to identify endogenous virus element (EVE) or provirus nucleic acid insertions in the genomic DNA.

In some embodiments, the method as disclosed herein to identify genomic safe harbor (GSH) regions in a mammalian genome, comprises (a) identifying the loci of the endogenous virus element (EVE) in the genomes of related species within taxonomic rank; (b) identifying the interspecific conserved loci in the human or mouse genome based on gene conservation or synteny; and functional validation of the candidate loci as a genomic safe harbor, e.g., functional validation in human and mouse progenitor and somatic cells (e.g., any of satellite cells, airway epithelial cells, any stem cell, induced pluripotent stem cells) using at least one or more in vitro or in vivo assays as disclosed herein. In some embodiments, functional validation of the candidate loci as a genomic safe harbor can be assessed in germline cells only in animal models and mice models at least one or more in vitro or in vivo assays as disclosed herein

In some embodiments, the functional assays are selected from any one or more of: (a) insertion of a marker gene into the loci in human cells and measure marker gene expression in vitro; (b) insertion of marker gene into orthologous loci in progenitor cells or stem cells and engraft the cells into immune-depleted mice and/or assess marker gene expression in all developmental lineages; (c) insertion of the marker gene into the GSH of undifferentiated hematopoietic CD34+ cells followed by applying cytokines to induce differentiation into terminally differentiated cell types, wherein the hematopoietic CD34+ cells have a marker gene inserted into the candidate GSH loci; or (d) generate transgenic knock-in mouse wherein the genomic DNA of the mouse has a marker gene inserted in the candidate GSH loci, wherein the marker gene is operatively linked to a tissue specific or inducible promoter.

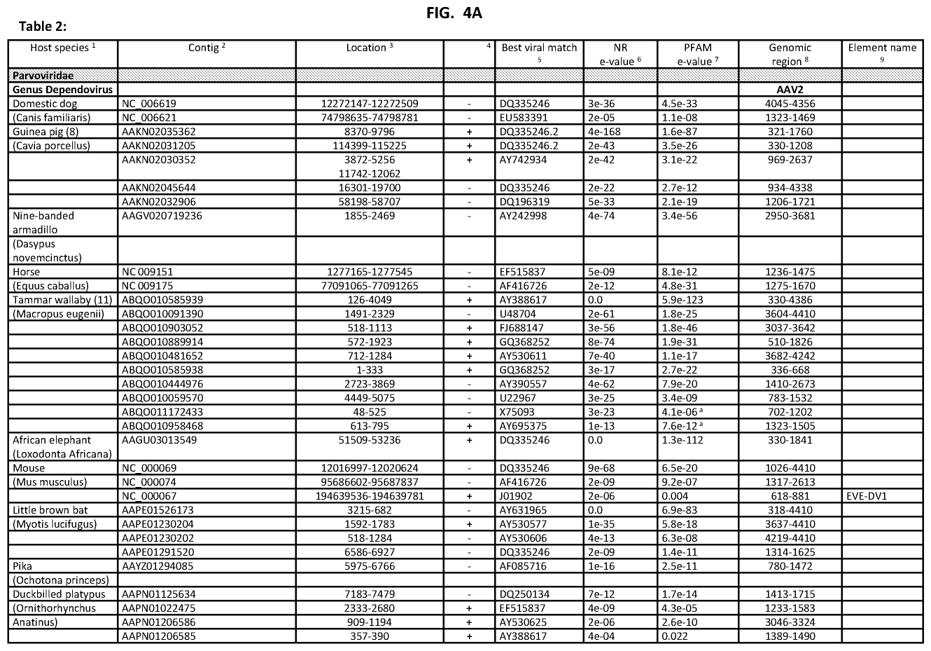

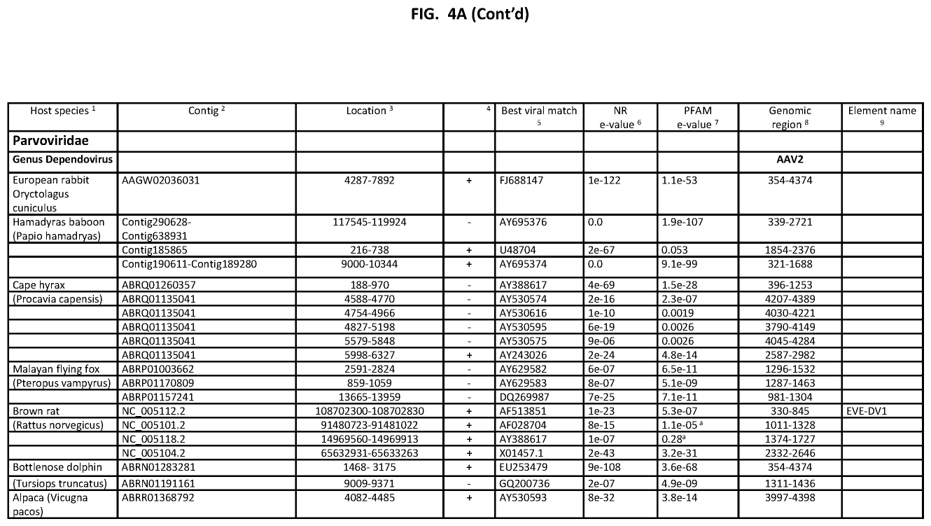

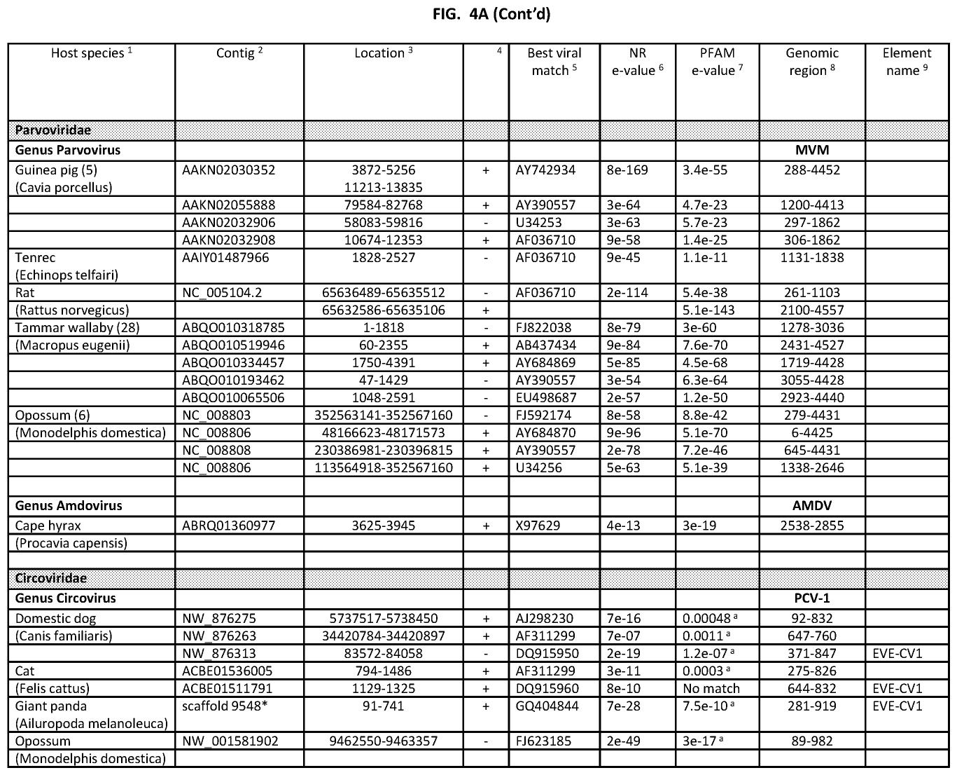

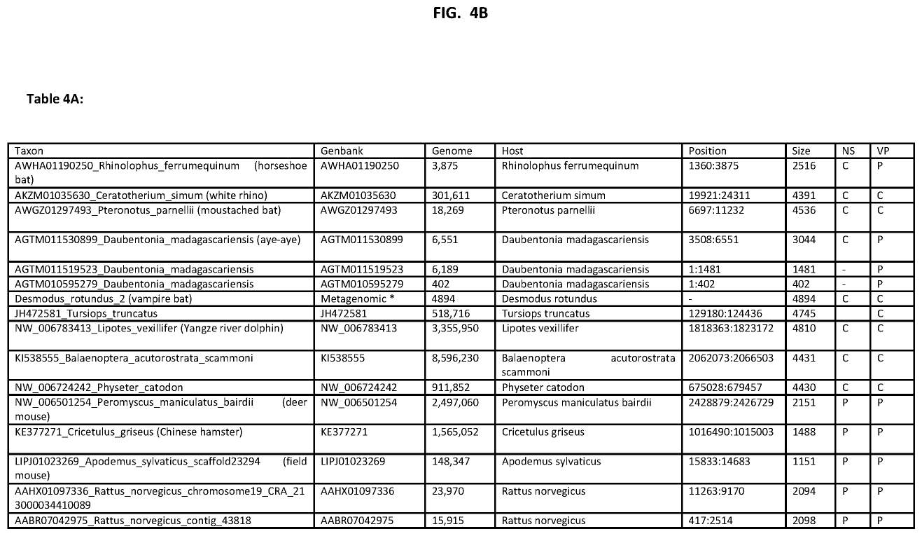

In some embodiments, the genome sequence of a model species is analyzed for the presence of the EVE. The model species can be from any phylogenetic taxa including, but not limited to: catacea, chiroptera, Lagomorpha, Macropodidae. Other model species be assessed, for example, rodentia, primates (except humans), monotremata. Other species can be used, for example, as listed in FIG. 4A, 4B of Lui et al., J Virology 2011; 9863-9876 which is incorporated herein in its entirety by reference.

In some embodiments, the EVE is a nucleic acid comprising intronic or exonic or intergenic viral nucleic acid, viral DNA, viral DNA or DNA copies of viral RNA. In some embodiments, the EVE comprises a region of viral nucleic acid from a non-retrovirus, i.e., the viral nucleic acid is non-retroviral viral nucleic acid.

In some embodiments, the EVE is a provirus, which is the virus genome integrated into the DNA of a non-virus host cell. In some embodiments, the EVE is a portion or fragment of the virus genome. In some embodiments, the EVE is a provirus from a retrovirus. In some embodiments, the EVE is not from a retrovirus. In some embodiments, the EVE is a provirus or fragment of a viral genome from a non-retrovirus.

In some embodiments, the EVE is nucleic acid from a parvovirus. The parvovirus family contains two subfamilies; Parvovirinae, which infect vertebrate hosts and Densovirinae, which infect invertebrate hosts. Each subfamily has been subdivided into several genera. In some embodiments, the EVE is a nucleic acid from a Densovirinae, from any of the following genus, densovirus, iteravirus, and contravirus.

In some embodiments, the EVE is a nucleic acid from a parvovirinae, from any of the following genera; Parvovirus, Erythrovirus, Dependovirus.

In some embodiments, the EVE is from the subfamily of Parvovirinae include the following genera:
a. Genus Amdoparvovirus: type species: Carnivore amdoparvovirus 1. Genus includes 2 recognized species, infecting mink and fox
b. Genus Aveparvovirus: type species: Galliform aveparvovirus 1. Genus includes a single species, infecting turkeys and chickens
c. Genus Bocaparvovirus: type species: Ungulate bocaparvovirus 1. Genus includes 12 recognized species, infecting mammals from multiple orders, including primates
d. Genus Copiparvovirus: type species: Ungulate copiparvovirus 1. Genus includes 2 recognized species, infecting pigs and cows
e. Genus Dependoparvovirus: type species: Adeno-associated dependoparvovirus A. Genus includes 7 recognized species, infecting mammals, birds or reptiles
f. Genus Erythroparvovirus: type species: Primate erythroparvovirus 1. Genus includes 6 recognized species, infecting mammals, specifically primates, chipmunk or cows
g. Genus Protoparvovirus: type species: Rodent protoparvovirus 1. Genus includes 5 recognized species, infecting mammals from multiple orders, including primates
h. Genus Tetraparvovirus: type species: Primate tetraparvovirus 1. Genus includes 6 recognized species, infecting primates, bats, pigs, cows and sheep

The Parvovirus subfamily is associated with mainly warm-blooded animal hosts. Of these, the RA-1 virus of the parvovirus genus, the B19 virus of the erythrovirus genus, and the adeno-associated viruses (AAV) 1-9 of the dependovirus genus are human viruses. In some embodiments, the EVE is from a virus that can infect humans, which are recognized in 5 genera: Bocaparvovirus (human bocavirus 1-4, HboV1-4), Dependoparvovirus (adeno-associated virus; at least 12 serotypes have been identified), Erythroparvovirus (parvovirus B19, B19), Protoparvovirus (Bufavirus 1-2, BuV1-2) and Tetraparvovirus (human parvovirus 4 G1-3, PARV4 G1-3).

In some embodiments, the EVE is from a parvovirus, and in some embodiments the EVE is nucleic acid from an AAV (adeno-associated virus). Adeno-associated virus (AAV), a member of the Parvovirus family, is a small nonenveloped, icosahedral virus with single-stranded linear DNA genomes of 4.7 kilobases (kb) to 6 kb. AAV is assigned to the genus, Dependoparvovirus, because the virus was discovered as a contaminant in purified adenovirus stocks, was originally designated as adenovirus associated (or satellite) virus. AAV's life cycle includes a latent phase at which AAV genomes, after infection, may integrate into host cell chromosomal DNA frequently at a defined locus, such as, e.g., AAV51, and a lytic phase in which, in which cells are co-infected with either adenovirus or herpes simplex virus and AAV, or superinfecting latent infected cells, the integrated genomes are subsequently rescued, replicated, and packaged into infectious viruses. Based on serological surveillance analyses, exposure to AAV is highly prevalent in humans and other primates and several serotypes have been isolated from various tissue samples. Serotypes 2, 3, and 6 were discovered in cultured human cells, and AAV5 was isolated from a clinical specimen, whereas AAV serotypes 1, 4, and 7-11 were isolated from nonhuman primate (NHP) tissue samples or cells. As of 2006 there have been 11 AAV serotypes described. Weitzman, et al., (2011). “Adeno-Associated Virus Biology”. In Snyder, R. O.; Moullier, P. Adeno-associated virus methods and protocols. Totowa, N.J.: Humana Press. ISBN 978-1-61779-370-7; Mori S, et al., (2004). “Two novel adeno-associated viruses from cynomolgus monkey: pseudotyping characterization of capsid protein”. Virology. 330 (2): 375-83).

In some embodiments, the EVE is a nucleic acid sequence, or part of a nucleic acid from any of the parvoviruses listed in Table 2 or Table 4A or Table 4B.

TABLE 4B
List of viruses in the parvovirinae
genus, and their accession numbers

| Parvovirinae | | Accession |
|---|---|---|
| Genus | Virus species or variant | number |
| Amdoparvovirus | Aleutian mink disease virus | JN040434 |
| | Gray fox amdovirus | JN202450 |
| Aveparvovirus | Aveparvovirus Turkey parvovirus | JN202450 |
| Bocaparvovirus | California sea lion bocavirus 1 | JN202450 |
| | Canine bocavirus 1 | JN648103 |
| | Canine minute virus | FJ214110 |
| | Feline bocavirus | JQ692585 |
| | Human bocavirus 1 | JQ692585 |
| | Human bocavirus 4 | FJ973561 |
| | Porcine bocavirus 1 | HM053693 |
| | Porcine bocavirus 3 | JF429834 |
| | Porcine bocavirus 5 | HQ223038 |
| Copiparvovirus | Bovine parvovirus 2 | AF406966 |
| | Porcine parvovirus 4 | GQ387499 |
| Dependoparvovirus | Adeno-associated virus 1 | GQ387499 |
| | Adeno-associated virus 2 | NC_001401 |
| | Adeno-associated virus 3 | NC001729 |
| | Adeno-associated virus 3B | NC_001863 |
| | Adeno-associated virus 4 | NC_001829 |
| | Adeno-associated virus 5 | AF085716 |
| | Adeno-associated virus 6 | NC_001862 |
| | Adeno-associated virus 7 | AF513851 |
| | Adeno-associated virus 8 | AF513852 |
| | Avian-AAV ATCC VR-865 | NC_004828 |
| | Avian-AAV ATCC DA-1 | NC_006263 |
| | Bat adeno-associated virus | GU226971 |
| | California sea lion adeno- | JN420372 |
| | associated virus 1 | |
| | Bovine AAV | NC_005889 |
| | Goose parvovirus | U25749 |
| Erythroparvovirus | Erythroparvovirus Human | M13178 |
| | parvovirus B19 | |
| Protoparvovirus | Bufavirus 1 | JX027296 |
| | Canine parvovirus | M19296 |
| | Mouse parvovirus 1 | U12469 |
| | Mouse parvovirus 3 | DQ196318 |
| | Porcine parvovirus PT4 | U44978 |
| | Rat parvovirus NTU1 | AF036710 |
| Tetraparvovirus | Bovine hokovirus | EU200669 |
| | Eidolon helvum parvovirus 1 | JQ037753 |
| | Human parvovirus 4 | AY622943 |
| | Porcine hokovirus | EU200677 |

In some embodiments, the EVE is nucleic acid from any serotype of AAV, including but not limited to AAV serotypes AAV1, AAV2, AAV3, AAV4, AAV5, AAV6, AAV7, AAV8, AAV9, AAV10 or AAV11 or AAV12.

In some embodiments, the EVE is a nucleic acid sequence from any of the group selected from: B19, minute virus of mice (MVM), RA-1, AAV, bufavirus, hokovirus, bocovirus, or any of the viruses listed in Table 2 or Table 4A or Table 4B, or variants thereof, that is, virus with 95%, 90%, 85%, or 80% nucleic acid or amino acid sequence identity.

In some embodiments, the EVE encodes the Rep and assembly activating non-structural (NS) proteins and structural (S) viral proteins (VP), for example, replication, capsid assembly, and capsid proteins, respectively. Such proteins include, but are not limited to, Rep (replication) proteins, including but not limited to Rep78, Rep68, Rep52, Rep40, and Cap (capsid) proteins, including but not limited to VP1, VP2 and VP3, e.g., from AAV. Structural proteins also include but are not limited to structural proteins A, B and C, for example, from AAV. In some embodiments, the EVE is a nucleic acid encoding all, or part of a non-structural (NS) protein or a structural (S) protein disclosed in Supplemental Table S2 in Francois, et al. “Discovery of parvovirus-related sequences in an unexpected broad range of animals.” Nature Scientific reports 6 (2016).

### B. Identifying Genomic Safe Harbors Using Comparative Genomic Approaches.

Another aspect of the technology described herein relates to a method to identify genomic safe harbors using comparative genomic approaches.

In particular, among evolutionary diverse species, the subchromosomal arrangement of genes often occur in a similar order (e.g., have collinearly) or as clustered loci (e.g., synteny). Analyzing the genomic collinearly and syntenic blocks can be used to determine whether sequence/gene loss or gain occurred within that region. Disrupting the genomic organization by the addition or loss of sequences or genes suggests a degree of flexibility in that subchromosomal region without affecting viability, cellular potency, ontogeny, etc.

Accordingly, in some embodiments, this approach may be applied to intergenic regions that lack coding sequences. By way of a non-limiting example, several cadherin genes are collinear in marsupial, rodent, and human species and the intergenic distance between the cadherin 8 and cadherin 11 genes are about 5.2 Mbp, 3.5 Mbp, and 2.9 Mbp, respectively. The interspecific sequence identity is limited to relatively short patches that may serve as genomic “bar-codes” to establish equivalent positions between species, within the intergenic space.

Phylogenetically, intronic sequences and spacing are more similar than intergenic sequences and spacing. Point mutations within introns are unlikely to affect genic functions except when occurring within several well characterized cis acting splicing elements within the intron, e.g., polypyrimidine tract or splice donor and acceptor signals. As a result of being embedded in genes, extensive perturbations of introns may disrupt transcript processing and translation efficiency, thus creating selective pressure for maintaining genic function.

Thus, a similar approach can be applied to interspecific intron comparison, where an enlarged intron in one species relative to another species identifies a potential genomic safe harbor.

Accordingly, one embodiment relates to a method to identify a GSH in a mammalian genome comprising comparing interspecific introns of collinearly organized or synteny organized genes to identify an enlarged intron in one species relative to another species. In some embodiments, an enlarged intron is identified as being an intron that larger by at least one sigma (σ) statistical difference, or preferably, at least two sigma (σ) or more statistical difference than the same intron in the gene of different species. As an exemplary example only, in an analysis of the introns of a selected gene in three different species, e.g., human, marsupial, and rodent species (where the selected gene is collinearly organized and/or synteny organized genes between the species), if the intron is larger (i.e., longer) in one species by at least one sigma statistical difference, or at least two statistically difference as compared to the same intron in the other species, it identified an enlarged intron and a potential site as a GSH.

By way of a non-limiting an example only, if an intron “al” of gene “A” in three different species, e.g., human, marsupial, or rodent species, is larger (i.e., longer) in one of the species by at least one sigma (σ) statistical difference or at least two sigma (σ) statistically difference, as compared to the same intron “al” in the other species, it identifies the intron “al” in gene “A” as enlarged intron and a potential site as a GSH.

In some embodiments, an enlarged intron is at least 20%, or at least 30%, or at least 40%, or at least 50%, or at least 60%, or at least 70%, or at least 80%, or at least 90%, or at least 100% larger, or between 20-50%, or between 50-80%, or between 80-100% larger than the comparative or corresponding intron in other species. In alternative embodiments, an enlarged intron is at least 1.2-fold, or at least about 1.4-fold, or at least about 1.5-fold, or at least about 1.6-fold, or at least about 1.8-fold, or at least about 2.0-fold, or at least about 2.2-fold, or at least about 2.4-fold, or at least about 2.5-fold or more than 2.5-fold larger (i.e., longer) than the comparative or corresponding intron in other species.

In another embodiment, a method to identify a GSH in a mammalian genome comprises comparing the intergenic distance (or space) between selected adjacent genes of collinearly organized or synteny organized genes in different species to identify large variations in the intergenic spaces between two genes in different species, and where there is a large variation in the intergenic space, it identifies a potential genomic safe harbor. Stated differently, if there is hypervariability between the distances (e.g., intergenic spaces) between two selected genes that are collinearly organized and/or synteny organized, it identifies a potential GSH. A hypervariable region is best described in that a region between genes selected genes “A” and “B” in different species varies greatly, where genes “A” and “B” are collinearly organized and/or synteny organized between species.

As an exemplary example, a large variation in the intergenic space or distance between two selected genes is at least 20%, or at least 30%, or at least 40%, or at least 50%, or at least 60%, or at least 70%, or at least 80%, or at least 90%, or at least 100% variability between different species. In some embodiments, a large variation in the intergenic space between two selected genes of collinearly organized and/or synteny organized genes between species, or a hypervariable region between genes is identified as a region that differs in size (e.g., length) by at least one sigma (σ) statistical difference, or preferably, at least two sigma (σ) or more statistical difference in three or more different species. As an exemplary example only, in an analysis of the intergenic space between to selected genes in three different species, e.g., human, marsupial, and rodent species (where the two selected genes that are collinearly organized and/or synteny organized genes between the species), if there is variation between the size (i.e., length) between the two selected genes in one species by at least one sigma (σ) statistical difference, or at least two statistically difference as compared to the size (i.e., length) between the same genes in at least one of other species, it identifies a large variation in intergenic space and a potential site as a GSH.

By way of a non-limiting example only, if genes A, B, C, D, E are collinearly organized and/or synteny organized genes between species, if one were to compare the distance between genes D and E, and the distances between A and B in different species, and if the distances between A and B are, for example, 10 kb, 50 kb and 45 kb in three different species, and the distances between gene D and E are, e.g., 1 kb, 1.5 kb and 1.2 kb in different species, it identified the intergenic distance or space between genes A and B as hypervariable and therefore, a potential GSH. In this example, the difference between the distance between genes A and B is 5-fold (e.g., 10 kb and 50 kb), whereas the difference between genes C and D is 1.5-fold (e.g., 1 kb and 1.5 kb), and the two-tailed P value between the distance between genes A-B and genes C-D is 0.0550, thus identifying the region between gene A and B having a large variation in intergenic space and a potential region as a GSH.

Preferably, one will preferably compare at least two intergenic spaces or distances between species of selected genes that are collinearly organized and/or synteny organized genes between species. For example, in the Example above, the intergenic space between genes A and B are compared with the intergenic space D and E, however, alternatively, one can compare the intergenic space between genes A and B, with the intergenic space between genes B and C etc. In some embodiments, a comparison of at least 2, or at least 3, or at least 4 intergenic spaces between genes in one will preferably compare at least two intergenic spaces that are collinearly organized and/or synteny organized between species is envisioned.

In another exemplary example, if genes A and B are collinearly organized and/or synteny organized genes between species, if one were to compare the distance between genes A and B in three or more different species (e.g., using ANOVA or other comparison methodology), and if the distance between A and B are statistically different, e.g., by at least one sigma statistical difference, or preferably, at least two sigma, in one species as compared to at least one other species, or both species, it identifies a large variation in intergenic space and a potential region as a GSH. In some embodiments, the intergenic spaces or distances between two selected genes of collinearly organized and/or synteny organized genes is assessed in at least 3, or at least 4, or at least 5, or at least 6 or at least 7 or at least 8 different species.

Accordingly, in some embodiments, the method as disclosed herein to identify genomic safe harbor (GSH) regions in a mammalian genome, comprises (a) comparative genomic approaches using (i) interspecific intron comparison to identify an enlarged intron between different species of a collinearly organized or synteny organized gene and/or (ii) intergenic space comparison to identify a large variation in the intergenic spaces between adjacent genes that are collinearly organized or synteny organized; (b) identifying the enlarged intron or variant intergenic space; and functional validation of the identified enlarge intron and/or variant intergenic space as a genomic safe harbor, e.g., functional validation in human and mouse progenitor and somatic cells (e.g., any of satellite cells, airway epithelial cells, any stem cell, induced pluripotent stem cells) using at least one or more in vitro or in vivo assays as disclosed herein. In some embodiments, functional validation of the identified enlarge intro and/or variant intergenic space as a genomic safe harbor can be assessed in germline cells only in animal models and mice models at least one or more in vitro or in vivo assays as disclosed herein.

### C. Optional Criteria for Selecting a GSH Loci or a Nucleic Acid Region of the GSH

In some embodiments, a GSH identified according to embodiments herein is an extragenic site that is remote from a known gene or a genomic regulatory sequence, or an intragenic site (within a gene) whose disruption is deemed to be tolerable.

In some embodiments, the GSH comprises may genes, including intragenic DNA comprising intronic and extronic gene sequences as well as intergenic or extragenic material.

In some embodiments, in addition to validating the identified GSH using functional in vitro and in vivo analysis as disclosed herein, a candidate GSH can be optionally assessed using bioinformatics, e.g., determining if the candidate GSH meets certain criteria, for example, but not limited to assessing for any one or more of the following: proximity to cancer genes or proto-oncogenes, location in a gene or location near the 5′ end of a gene, location in selected housekeeping genes, location in extragenic regions, proximity to mRNA, proximity to ultra-conserved regions and proximitiy to long noncoding RNAs and other such genomic regions.

By way of Example, the previously identified GSH AAV51 (adeno-associated virus integration site 1), was identified as the adeno-associated virus common integration site on chromosome 19 and is located in chromosome 19 (position 19q13.42) and was primarily identified as a repeatedly recovered site of integration of wild-type AAV in the genome of cultured human cell lines that have been infected with AAV in vitro. Integration in the AAV51 locus interrupts the gene phosphatase 1 regulatory subunit 12C (PPP1R12C; also known as MBS85), which encodes a protein with a function that is not clearly delineated. The organismal consequences of disrupting one or both alleles of PPP1R12C are currently unknown. No gross abnormalities or differentiation deficits were observed in human and mouse pluripotent stem cells harboring transgenes targeted in AAV51. Previous assessment of the AAV51 site typically used Rep-mediated targeting which preserved the functionality of the targeted allele and maintained the expression of PPP1R12C at levels that are comparable to those in non-targeted cells. AAV51 was also assessed using ZFN-mediated recombination into iPSCs or CD34+ cells.

As originally characterized, the AAV51 locus is >4 kb and is identified as chromosome 19 nucleotides 55,113,873-55,117,983 (human genome assembly GRCh38/hg38) and overlaps with exon 1 of the PPP1R12C gene that encodes protein phosphatase 1 regulatory subunit 12C. This >4 kb region is extremely G+C nucleotide content rich and is a gene-rich region of particularly gene-rich chromosome 19 (see FIG. 1A of Sadelain et al., Nature Revs Cancer, 2012; 12; 51-58), and some integrated promoters can indeed activate or cis-activate neighboring genes, the consequence of which in different tissues is presently unknown.

AAV51 GSH was identified by characterizing the AAV provirus structure in latently infected human cell lines with recombinant bacteriophage genomic libraries generated from latently infected clonal cell lines (Detroit 6 clone 7374 IIID5) (Kotin and Berns 1989), Kotin et al., isolated non-viral, cellular DNA flanking the provirus and used a subset of “left” and “right” flanking DNA fragments as probes to screen panels of independently derived latently infected clonal cell lines. In approximately 70% of the clonal isolates, AAV DNA was detected with the cell-specific probe (Kotin et al. 1991; Kotin et al. 1990). Sequence analysis of the pre-integration site identified near homology to a portion of the AAV inverted terminal repeat (Kotin, Linden, and Berns 1992). Although lacking the characteristic interrupted palindrome, the AAV51 locus retained the p5 Rep proteins binding and nicking, also referred to as the terminal resolution sites (Chiorini et al. 1994; Chiorini et al. 1995; Im and Muzyczka 1989, 1990, 1992). Interestingly, the human orthologue functioned as a p5 Rep in vitro origin of DNA synthesis, thus supporting the early conjecture that AAV51 integration is a Rep-dependent process (Kotin et al., 1990; Kotin et al., 1992; Urcelay et al. 1995; Weitzman et al. 1994). The Rep binding elements in cis were shown to be required for AAV integration and providing additional support for Rep protein involvement in the targeted, non-homolgous recombination process (Urabe, et al., Linden . . . Berns). These elements define the minimum origin of Rep-mediated DNA synthesis as the arrangement of Rep binding and nicking sites that allow RNA-primer independent strand-displacement DNA (leading strand) synthesis.

The wild-type adeno-associated virus may cause either a productive or latent infection, where the wild-type virus genome integrates frequently in the AAV51 locus on human chromosome 19 in cultured cells (Kotin and Berns 1989; Kotin et al. 1990). This unique aspect of AAV has been exploited as one of the first so-called “safe-harbors” for iPSC genetic modification. AAV51, as originally defined (Kotin et al., 1991) is situated on chromosome 19 between nucleotides 55,113,873-55,117,983 (human genome assembly GRCh38/hg38) and overlaps with exon 1 of the PPP1R12C gene that encodes protein phosphatase 1 regulatory subunit 12C. Interesting, PPP1R12C exon 1 5′untranslated region contains a functional AAV origin of DNA synthesis indicated within the following sequences (Urcelay et al. 1995): The initiation methionine codon is underlined, the GCTC Rep-binding motifs and terminal resolution site (GGTTGG) are indicated with bold font: 55,117,600-TGGTGGCGGCGGTTGGGGCTCGGCGCTCGCTCGCTCGCTCGCTGGGCGGGCGGTGCGATG-55,117,540.

Surprisingly, the human chromosome 19 AAV51 safe-harbor is within an exonic region of PPP1R12C, the gene encoding protein phosphatase regulatory 1 regulatory subunit 12C. The selection of the exonic integration site is non-obvious, and perhaps counter-intuitive, since insertion and expression of foreign DNA will likely disrupt the expression of the endogenous genes. Apparently, insertion of the AAV genome into this locus does not adversely affect cell viability or iPSC differentiation (DeKelver et al. 2010; Wang et al. 2012; Zou et al. 2011). Integration occurs by non-homologous recombination that requires the presence of AAV Rep proteins in trans and the minimum origin of AAV DNA synthesis in cis on both recombination substrates which then permits Rep-protein mediated juxtapositioning of the AAV and genomic DNAs (Weitzman et al. 1994).

The Rep-dependent minimum origin of DNA synthesis consists of the p5 Rep protein binding elements (RBE) and properly positioned terminal resolution site (trs) as exemplified by the AAV2 trs AGT1TGG and the AAV5 trs AGTG1TGG (the vertical line indicates the nicking position). In addition, the involvement of cell protein complexes has been inferred, but not yet identified or characterized.

These virus replication elements must function very efficiently or the virus would become extinct due to lack of replicative fitness, whereas, the small, non-coding, ca. 35 bp element in AAV51 may have no function in the host. However, the AAV51 locus has been established as a somatic cell safe harbor and disruption of the locus in totipotent or germline cells may interfere with ontogeny.

The AAV51 locus is within the 5′ UTR of the highly conserved PPP1R12C gene. The Rep-dependent minimal origin of DNA synthesis is conserved in the 5′UTR of the human, chimapanzee, and gorilla PPP1R12C gene. However, in rodent species (mouse and rat), substitutions occur with increased frequency within the preferred terminal resolution site compared to adjacent non-coding DNA. The incidental rather than selected or acquired genotype may affect the efficiency of the other species the specific sequences in the 5′ UTR.

In some embodiments, a candidate GSH identified according to embodiments herein is identified to meet the criteria of a GSH if it is safe and targeted gene delivery can be achieved that has limited off-target activity and minimal risk of genotoxicity, or causing insertional oncogenesis upon integration of foreign DNA, while being accessible to highly specific nucleases with minimal off-target activity.

While the GSH is validated based on in vitro and in vivo assays as described herein, in some embodiments, additional selection can be used based on determining whether the GSH falls into a particular criterion. For example, in some embodiments, a GSH loci identified herein is located in an exon, intron or untranslated region of a dispensable gene. Analysis shows that integration sites of provirus in tumors commonly lie near the starting point of transcription, either upstream or just within the transcription unit, often within a 5′ intron. Proviruses at these locations have a tendency to dysregulate expression by increasing the rate of transcription either via virus promoter or via virus enhancer insertions. Accordingly, in some embodiments, a GSH locus identified herein is selected based on not being proximal to a cancer gene. In some embodiments, a GSH does not have an integration site located near the starting point of transcription of a cancer gene, e.g. upstream or in the 5′ intron of a cancer gene or proto-oncogene. Such cancer genes are well known to one of ordinary skill in the art, and are disclosed in Table 1 in Sadelain et al., Nature Revs Cancer, 2012; 12; 51-58, which is incorporated herein in its entirety. Exemplary databases of genes implicated in cancer are well known, e.g., Atlas gene set, CAN gene sets, CIS (RTCGD) gene set, and described in Table 5 below:

TABLE 5

| | Number | | | |
|---|---|---|---|---|
| Gene set* | of genes | Species | Description | Refs |
| Atlas | 999 | human | This gene set is from the Atlas of genetics and cytogenetics in | 41 |
| | | | oncology and hematology. | |
| | | | It lists both hybrid genes found in at least one cancer case and | |
| | | | gene amplifications or | |
| | | | homozygous deletions found in a significant subset of cases in a | |
| | | | given cancer type | |
| Miscellaneous | 187 | Multiple | This gene set is from Retroviruses (Cold Spring Harbor | 35 |
| | | | Laboratory Press), an early version of the CIS database, a list | |
| | | | from T. Hunter, The Salk Institute. La Jolla, California, USA, and | |
| | | | miscellaneous additions from the scientific literature | |
| CAN genes | 192 | | This gene set includes 192 common genes that were mutated at | 42 |
| | | | significant frequency in all tumors of human breast and | |
| | | | colorectal cancers | |
| CIS | 593 | Mouse | This gene set is from the Mouse Variation Resource and lists | 36 |
| (RTCGD) | | | retroviral insertional mutagenesis in mouse hematopoietic tumors | |
| Human | 38 | Human | This gene set is a list of lymphoid-specific oncogenes that was | |
| lymphoma | | | compiled by M. Cavazzana-Calvo and colleagues, Hôpital | |
| | | | Necker, Paris, France | |
| Sanger | 452 | Human | This gene set is from the Cancer Gene Census, a compilation | 43 |
| | | | from the scientific literature of “mutated genes that are causally | |
| | | | implicated in oncogenesis.” | |
| Waldman | 455 | Human | This gene set is from the Waldman gene database and lists cancer | |
| | | | genes sorted by chromosomal locus and includes links to OMIM | |
| AllOnco | 2,070 | Mouse and | This database is a master set of the seven sets described above in | |
| | | human | which all genes are converted to their human homologues | |

*Gene lists and links to original sources are available at The Bushman lab cancer gene list website (see Further information). CAN, cancer; CIS, common insertion site; References in the last column represent the reference number in Sadelain et al., Nature Revs Cancer. 2012; 12; 51-58.

In some embodiments, a GSH loci identified herein has any or more of the following properties: (i) outside a gene transcription unit; (ii) located between 5-50 kilobases (kb) away from the 5′ end of any gene; (iii) located between 5-300 kb away from cancer-related genes; (iv) located 5-300 kb away from any identified microRNA; and (v) outside ultra-conserved regions and long noncoding RNAs. In some embodiments, a GSH locus identified herein has any or more of the following properties: (i) outside a gene transcription unit; (ii) located >50 kilobases (kb) from the 5′ end of any gene; (iii) located >300 kb from cancer-related genes; (iv) located >300 kb from any identified microRNA; and (v) outside ultra-conserved regions and long noncoding RNAs. In studies of lentiviral vector integrations in transduced induced pluripotent stem cells, analysis of over 5,000 integration sites revealed that ˜17% of integrations occurred in safe harbors. The vectors that integrated into these safe harbors were able to express therapeutic levels of β-globin from their transgene without perturbing endogenous gene expression.

II. Functional Validation of a Candidate GSH Using In Vitro and In Vivo Assays

While not being limited to theory, a useful GSH region must permit sufficient transgene expression to yield desired levels of the vector-encoded protein or non-coding RNA, and should not predispose cells to malignant transformation nor significantly negatively alter cellular functions.

Methods and compositions for validating the candidate GSH regions disclosed herein include, but are not limited to; bioinformatics, in vitro gene expression assays, in vitro and in vivo expression arrays to query nearby genes, in vitro-directed differentiation or in vivo reconstitution assays in xenogeneic transplant models, transgenesis in syntenic regions and analyses of patient databases from individuals.

In one embodiment, the validation of the GSH is determined to check that there is no germline integration of the introduced gene, reducing risks that there is germline transmission of the gene therapy vector.

Following identification of a target loci or candidate GSH, a series of in vitro and in vivo assays can be used to establish safety and in particular, the absence of oncogenic potential. In vitro oncogenicity assays can be based on the experience in previous gene therapy T-cell product characterizations.

### A. In Vitro Assays to Validate the GSH

In some embodiments, the GSH can be validated by a number of assays. In some embodiments, functional assays are selected from any one or more of: (a) insertion of a marker gene into the loci in human cells and measure marker gene expression in vitro; (b) insertion of marker gene into orthologous loci in progenitor cells or stem cells and engraft the cells into immunodepleted mice and/or assess marker gene expression in all developmental lineages; (c) differentiate hematopoietic CD34+ cells into terminally differentiated cell types, wherein the hematopoietic CD34+ cells have a marker gene inserted into the candidate GSH loci; or (d) generate transgenic knock-in mouse wherein the genomic DNA of the mouse has a marker gene inserted in the candidate GSH locus, wherein the marker gene is operatively linked to a tissue specific or inducible promoter.

In some embodiments, a functional assay to validate the GSH involves insertion of a marker gene into the loci of a human cell and determination of expression of the marker in vitro. In some embodiments, the marker gene is introduced by homologous recombination. In some embodiments, the marker gene is operatively linked to a promoter, for example, a constitutive promoter or an inducible promoter. The determination and quantification of gene expression of the marker gene can be performed by any method commonly known to a person of ordinary skill in the art, e.g., gene expression using e.g., RT-PCR, Affymetrix gene array, transcriptome analysis; and/or protein expression analysis (e.g., western blot) and the like. In some embodiments, the effect of the integrated marker transgene on neighboring gene expression is determined in cultured cells in vitro.

In some embodiments, the cell the marker gene is introduced into is a mammalian cell, e.g., a human cell or a mouse cell or a rat cell. In some embodiments, the cell is a cell line, e.g., a fibroblast cell line, HEK293 cells and the like. In some embodiments, the cell used in the assay are pluripotent cells, e.g., iPSCs or clonable cell types, such as T lymphocytes. In some embodiments, the gene expression of the insertion of a marker gene into a variety of different cell populations, including primary cells is assessed. In some embodiments, a iPSC that has an introduced marker gene is differentiated into multiple lineages to check consistent and reliable gene expression of the marker gene in different lineages.

In some embodiments, a marker gene is inserted into a candidate GSH loci in the genome of hematopoietic cells, such as, for example, CD34+ cells, and differentiated into different terminally differentiated cell types.

In some embodiments, a cell population that has a marker gene introduced into the candidate GSH can be assessed for possible tissue malfunction and/or transformation. For example, a CD34+ cells or iPSCs are assessed for aberrant differentiation away from normal lineage differentiation, and/or increased proliferation which would indicate a risk of cancer.

In some embodiments, the gene expression levels of proximal genes are determined. For instance, in some embodiments, if the integrated marker gene results in aberrant gene expression of surrounding or neighboring gene expression, or other dysregulation, such as a downregulation or upregulation of gene expression of the neighboring genes, the candidate loci is not selected as a suitable GSH. In some embodiments, if no change is detected in the expression level of a neighboring gene, the candidate loci is nominated, or selected, as a GSH. In some embodiments, the gene expression of flanking, proximal or neighboring genes is determined, where a proximal or neighboring gene can be within about 350 kb, or about 300 kb, or about 250 kb or about 200 kb or about 100 kb, or between 10-100 kb, or between about 1-10 kb or less than 1 kb distance (upstream or downstream) from the site of insertion of the marker gene (i.e., genes or RNA sequences flanking either in the 5′ or 3′ of the insertion loci).

In some embodiments, the epigenetic features and profile of the targeted candidate GSH loci is assessed before and after introduction of the marker gene to determine whether the introduction of the marker gene affects the epigenetic signature of the GSH, and/or surrounding or neighboring genes within about 350 kb upstream and downstream of the site of integration.

In some embodiments, insertion of a marker gene into a candidate GSH loci is assessed to see if the loci can accommodate different integrated transcription units. In some embodiments, the gene expression of a marker gene operatively linked to a range of different genetic elements, including promoters, enhancers and chromatin determinants, including locus control regions, matrix attachments regions and insulator elements) is assessed, as well as, in some embodiments, the gene expression of neighboring genes within about 350 kb, or about 300 kb, or about 250 kb or about 200 kb or about 100 kb, or between 10-100 kb, or between about 1-10 kb or less than 1 kb distance (upstream or downstream) from the site of insertion of the marker gene.

In some embodiments, where a GSH loci is associated with a specific gene, knock-down of the gene can be assessed to validate that the gene is either not necessary or is dispensable. As an exemplary example, one candidate GSH is the PAX5 gene (also known as Paired Box 5, or “B-cell lineage specific activator protein,” or BSAP). In humans PAX5 is located on chromosome 9 at 9p13.2 and has orthologues across many vertebrate species, including, human, chimp, macaque, mouse, rat, dog, horse, cow, pig, opossum, platypus, chicken, lizard, xenopus, C. elegans, drosophila and zebrafish. PAX5 gene is located at Chromosome 9: 36,833,275-37,034,185 reverse strand (GRCh38:CM000671.2) or 36,833,272-37,034,182 in GRCh37 coordinates.

PAX5 gene is surrounded by several different coding genes and RNA genes, as shown in FIG. 1. Accordingly, in one embodiment, the effect on the cell function and gene expression of neighboring cells on RNAi knockdown of PAX5 could be assessed, and where knock-down of the candidate gene in the GSH loci does not have significant effect, the gene can be identified as a GSH. Also, in vitro assays using RNAi to knock-out the GSH gene are important to determine the dispensability of the disrupted gene, especially resulting from biallelic disruption, as is often the case with endonuclease-mediated targeting.

In some embodiments, because cancer chemotherapy cytotoxic agents have genotoxic and carcinogenic potential, standard in vitro studies for preclinical evaluations of these types of drugs can also be used to assess GSH locus disruption. For example, the ability of a primary T cell to grow without cytokines and cell signaling is a feature of carcinogenic transformation.

For example, in some embodiments, one can introduce the marker gene into the candidate GSH loci of T-cells, e.g., SB-728-T cells and culture without cytokine support for several weeks and demonstrate that normal cell death occurs.

In another embodiment, the classic biological cell transformation assay is anchorage-independent growth of fibroblasts and is a stringent test of carcinogenesis. Accordingly, in some embodiments, a marker gene can be inserted into a target GSH loci in fibroblasts and assessed for anchorage-independent growth. Other in vitro assays or tests for evaluating oncogenicity can be used, e.g., mouse micronucleus test, anchorage independent growth, and mouse lymphoma TK gene mutation assay.

In some embodiments, the marker gene is selected from any of fluorescent reporter genes, e.g., GFP, RFP and the like, as well as bioluminescence reporter genes. Exemplary marker genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreenl), HcRed, DsRed, cyan fluo-rescent protein (CFP), yellow fluorescent proteins (e.g., YFP, EYFP, Citrine, Venus YPet, PhiYFP, ZsYellowl), cyan fluorescent proteins (e.g., ECFP, Cerulean, CyPet AmCyanl, Midoriishi-Cyan) red fluorescent proteins (e.g., mKate, mKate2, mPlum, DsRed monomer, mCherry, mRFP1, DsRed-Express, DsRed2, HcRed-Tandem, HcRedl, AsRed2, eqFP611, mRaspberry, mStrawberry, Jred), orange fluorescent proteins (e.g., mOrange, mKO, Kusabira-Orange, monomeric Kusabira-Orange, mTangerine, tdTomato) and autofluorescent proteins including blue fluorescent protein (BFP).

In some embodiments, the marker gene, or reporter gene sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. In some embodiments, where the marker gene is green fluorescent protein or luciferase, the vector carrying the signal may be measured colorimetrically based on visible light absorbance or light production in a luminometer, respectively. Such reporters can, for example, be useful in verifying the tissue-specific targeting capabilities and tissue specific promoter regulatory activity of a nucleic acid.

In some embodiments, bioinformatics can be used to validate the GSH, for example, reviewing sequences of databases of patient-derived autologous iPSC, as described in Papapetrou et al., 2011, Na. Biotechnology, 29; 73-78, which is incorporated herein in its entirety.

Additionally, once a GSH and target integration site in GSH is identified, bioinformatics and or web-based tools can be used to identify potential off-target sites. For example, bioinformatics tools such as Predicted Report of Genome-wide Nuclease Off-Target Sites (PROGNOS, http://baolab.bme.gatech.edu/Research/BioinformaticTools/prognos.html) and CRISPOR (http://crispor.tefor.net/) for designing CRISPR/Cas9 target and predicting off-target sites. CRISPOR and PROGNOS can provide a report of potential genome-wide nuclease target sites for ZFNs and TALENs. Once a particular target site is identified, the programs can provide a list ranking potential off-target sites.

### B. In Vivo Assays to Validate the GSH

In some embodiments, in vivo assays to functionally validate the GSH should be done in parallel with in vitro assays. In some embodiments, in vivo evaluation of GSHs can be performed in transgenic mice bearing a transgene that are integrated into syntenic regions.

In some embodiments, an in vivo functional assay to validate the GSH involves insertion of a marker gene into the loci of a iPSC and transplantation to immunodeficient mice. In some embodiments, the insertion of a marker gene into a iPSC and the modified iPSC implanted into immunodeficient mice and assessed over a period of time. Such an in vivo assay allows any genotoxic event to be assessed, including atypical or aberrant differentiation (e.g., changes in hematopoietic transformation and/or clonal skewing of hematopoiesis), as well as the outgrowth of tumorigenic cells to be assessed from a rare event.

Such in vivo methods in immunodeficient mice with hematopoietic cells are well known to one of ordinary skill in the art, and are disclosed in Zhou, et al. “Mouse transplant models for evaluating the oncogenic risk of a self-inactivating XSCID lentiviral vector.” PloS one 8.4 (2013): e62333, which is incorporated herein in its entirety by reference, where the malignancy incidence from the introduced modified hematopoeitc cells or iPSC can be assessed as compared to control or cells where no marker gene is introduced at the target loci in the GSH. In some embodiments, hematopoietic malignancy can be assessed. In some embodiments, lineage distribution of peripheral blood cells in the recipient immunodeficient mice is assessed to determine myeloid skewing and a signal of insertional transformation or adverse effects due to the marker gene inserted at the GSH loci.

In some embodiments, because the recipient mouse strains are immunodeficient, if tumors do arise in such mice, one can characterize these tumors and evaluate whether they are of human origin. If tumors are of human origin, then it will be necessary to further evaluate their clonality with respect to the insertion of the marker gene at the GSH loci or any dysregulation gene expression (upregulation or downregulation) of on- or off-target sites, such as flanking RNA sequences or genes. However, clonality observed in a marker-gene introduced cell does not necessarily equal causality and may instead be an innocent label that merely reflects the tumor's clonal origin.

In some embodiments, in vivo assays can be used that rely on the fact that human T cells can be maintained in immunodeficient NOG mice. Such an assay requires the marker gene to be introduced into the target GSH loci and modified human T cells allowed to live and expand for months in the NOG model, and compared to non-modified T cells. In some embodiments, a model with human T-cell xeno-GVHD can be used, where 2 months is allowed for a maximal time for proliferation of cells before animals died of GVHD, and defining a dose and donors that gave reliable GVHD in the NOG mice. After 2 months, the animals are euthanized and tissues evaluated by histology for neoplasms, immunostaining to detect human cells, and gene expression analysis (e.g., Affymetrix array or RT-PCR of flanking genes surrounding the GSH insertion loci) for detection of modified gene expression of on-target and off-target sites.

In some embodiments, another in vivo assay to functionally validate the candidate loci as a GSH is generating knock-in transgenic animals or transgenic mice.

Testing for Successful Gene Editing into a GSH of an iPSC or T-Lymphocyte or Other Host Cell

Assays well known in the art can be used to test the efficiency of insertion of the marker gene in both in vitro and in vivo models. Expression of the marker gene can be assessed by one skilled in the art by measuring mRNA and protein levels of the desired transgene (e.g., reverse transcription PCR, western blot analysis, and enzyme-linked immunosorbent assay (ELISA)). In one embodiment, the expression of the marker or reporter protein that can be used to assess the expression of the desired transgene, for example by examining the expression of the reporter protein by fluorescence microscopy or a luminescence plate reader. For in vivo applications, protein function assays can be used to test the functionality of a given gene and/or gene product to determine if gene editing has successfully occurred. It is contemplated herein that the effects of gene editing in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

### III. Nucleic Acid Constructs, and Kits for Targeting Homologous Recombination at a GSH Loci

As described above, nucleases specific for the safe harbor genes can be utilized such that the transgene construct is inserted by either HDR- or NHEJ-driven processes.

### A. Vectors Comprising a Portion of the GSH Loci

In some embodiments, the disclosure herein relates to nucleic acid vector compositions, e.g., a nucleic acid vector composition comprising at least a portion or region of the GSH identified using the methods disclosed herein. The portion or region of the GSH can be modified, e.g., where a point mutation can disrupt or knock-out the gene function of the GSH gene identified herein. In other embodiments, the portion or region of the GSH in the vector can be modified to comprise a guide RNA (gRNA) inserted, e.g., a guide RNA for a nuclease as disclosed herein. In some embodiments, the GSH vector can comprise a target site for a guide RNA (gRNA) as disclosed herein, or alternatively, a restriction cloning site for introduction of a nucleic acid of interest as disclosed herein. In another embodiment, a recombinase recognition site such as loxP may be introduced to facilitate directed recombination using a Cre recombinase expressed from rAAV or other gene transfer vector. The loxP site inserted into the GSH may also be used by breeding with tg mice that express Cre in a tissue specific manner.

In all aspects as disclosed herein, the nucleic acid vector compositions can be a plasmid, cosmid, or artificial chromosome (e.g., BAC), minicircle nucleic acid, or recombinant viral vector (e.g., rAd, AAV, rHSV, BEV or variants thereof). In some embodiments, the vector can comprise recombinase recognition sites (RRS), for example, LoxP sites, attP, AttB sites and the like.

One aspect of the technology described herein relates to a recombinant nucleic acid comprising at least a portion of the GSH nucleic acid identified as a genomic safe harbor (GSH) in the methods described herein. For example, in some embodiments, the recombinant nucleic acid is present in a vector, e.g., a plasmid, cosmid or artificial chromosome, such as, for example, a BAC. In some embodiments, the nucleic acid composition comprises at least a target site of integration in a GSH, and 5′ and 3′ portions of the GSH nucleic acid flanking the target site of integration.

In some embodiments, the recombinant nucleic acid composition comprises a GSH nucleic acid sequence is between 30-1000 nucleotides, between 1-3 kb, between 3-5 kb, between 5-10 kb, or between 10-50 kb, between 50-100 kb, or between 100-300 kb or between 100-350 kb in size, or any integer between 30 base pairs and 350 kb.

In some embodiments, the recombinant nucleic acid composition comprises a nucleic acid sequence comprising a first nucleic acid sequence comprising a 5′ region of the GSH, and a second nucleic sequence comprising a 3′ region of the GSH. In some embodiments, the 5′ region is within close proximity and upsteam of a target site of integration and the 3′ region of the GSH is in close proximity and downstream of a target site of integration.

In some embodiments, the recombinant nucleic acid composition comprises at least a portion of the PAX5 human genomic DNA or a fragment thereof, wherein the PAX5 is located at Chromosome 9: 36,833,275-37,034,185 reverse strand (GRCh38.p7:CM000671.2) or 36,833,272-37,034,182 in GRCh37 coordinates (see FIG. 1). In some embodiments, the recombinant nucleic acid composition comprises a nucleic acid sequence corresponding to at least a portion of untranslated a sequence or an intron of the PAX5 gene. In some embodiments, the untranslated sequence is a 5′UTR or 3′UTR of the PAX5 gene.

In some embodiments, the recombinant nucleic acid sequence comprises the genomic nucleic acid sequence, or a portion thereof, of any of the genes listed in Table 1A and Table 1B herein.

B. Vectors for Integration of a Nucleic Acid of Interest into a GSH Loci

In alternative embodiments, the disclosure herein also relates to nucleic acid vector compositions comprising at GSH-5′ homology arm, and a 3′GSH homology arm flanking a nucleic acid comprising a restriction cloning site, where the vector can be used to integrate the flanked nucleic acid into the genome at a GSH by homologous recombination. In all aspects as disclosed herein, the nucleic acid vector compositions can be a plasmid, cosmid, or artificial chromosome (e.g., BAC), minicircle nucleic acid, or recombinant viral vector (e.g., rAd, AAV, rHSV, BEV or variants thereof).

Accordingly, one aspect of the technology described herein relates to a nucleic acid vector composition comprising: (a) a GSH 5′ homology arm (also referred to herein as “5′ GSH-specific homology arm” or “5′ GSH-HA”), (b) a nucleic acid sequence comprising a restriction cloning site, and (c) a GSH 3′ homology arm (also referred to herein as “3′ GSH-specific homology arm” or “3′ GSH-HA”), where the 5′ homology arm and the 3′ homology arm bind to a target site located in a genomic safe harbor locus identified according to the methods as disclosed herein, and wherein the 5′ and 3′ homology arms allow insertion (of the nucleic acid located between the homology arms) by homologous recombination into a loci located within the genomic safe. In some embodiments, a nucleic acid vector composition for integration of a nucleic acid of interest into a GSH loci comprises a nucleic acid of interest and/or an expressible transgene cassette (e.g., a sequence that encodes a gene editing molecule described herein, or a reporter protein), The vectors can comprise e.g., one or more gene editing molecules.

In some embodiments, a nucleic acid vector composition for integration of a nucleic acid of interest into a GSH loci as described herein comprises in this order: a) a 5′ GSH-specific homology arm, c) a restriction cloning site, and d) a 3′ GSH-specific homology arm—

In some embodiments, the 3′ and 5′ homology arms complementary base pair with regions of the GSH identified according to the methods as disclosed herein. In some embodiments, 3′ and 5′ homology arms flank a target site of integration, e.g., target insertion loci in the GSH as disclosed herein. In some embodiments, the 3′ homology arm complementary base pairs with a nucleic acid region 3′ (i.e., upstream) of a target site of integration or target insertion loci of the GSH, and 5′homology arm complementary base pairs with a nucleic acid region 5′ (i.e., downstream) of a target site of integration or target insertion loci of the GSH. In some embodiments, the 5′ and 3′ homology arms are complementary to, e.g., at least 60%, or at least 70%, or at least 80%, or at least 85%, or at least 90%, or at least 91%, or at least 92%, or at least 93%, or at least 94%, or at least 94%, or at least 96%, or at least 97%, or at least 98%, or at least 99%, or at least 99.5% complementary to portions of the GSH identified herein.

For integration of the nucleic acid located between the 5′ and 3′ homology arms of the vector, the 5′ and 3′ homology arms should be long enough for targeting to the GSH and allow (e.g., guide) integration into the genome by homologous recombination. For example, a nucleic acid vector composition for integration of a nucleic acid of interest into a GSH loci as described herein vector may contain nucleotides encoding 5′ and 3′ homology arms for directing integration by homologous recombination into the genome of the host cell at a precise location(s) in the GSH identified herein.

To increase the likelihood of integration at a precise location, the 5′ and 3′ homology arms may include a sufficient number of nucleic acids, such as 50 to 5,000 base pairs, or 100 to 5,000 base pairs, or 500 to 5,000 base pairs, which have a high degree of sequence identity or homology to the corresponding target sequence to enhance the probability of homologous recombination. The 5′ and 3′ homology arms may be any sequence that is homologous with the GSH target sequence in the genome of the host cell. That is, the 5′ and 3′ homology arms are complementary to portions of the GSH target sequence identified herein. Furthermore, the 5′ and 3′ homology arms may be non-encoding or encoding nucleotide sequences. In some embodiments, the homology between the 5′ homology arm and the corresponding sequence on the chromosome is at least any of 80%, 85%, 90%, 95%, 97%, 98%, 99%, or 100%. In embodiments, the homology between the 3′ homology arm and the corresponding sequence on the chromosome is at least any of 80%, 85%, 90%, 95%, 97%, 98%, 99%, or 100%. In embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 100, 200, 300, 400, or 500 bp away from the integration or DNA cleavage site, or partially or completely overlapping with the DNA cleavage site. In embodiments, the 3′ homology arm of the nucleotide sequence is proximal to the altered ITR.

In some embodiments, the 5′ and/or 3′ homology arm can be any length, e.g., between 30-2000 bp. In some embodiments, the 5′ and/or 3′ homology arms are between 200-350 bp long. Details study regarding length of homology arms and recombination frequency is e.g., reported by Zhang et al. “Efficient precise knockin with a double cut HDR donor after CRISPR/Cas9-mediated double-stranded DNA cleavage.” Genome biology 18.1 (2017): 35, which is incorporated herein in its entity by reference.

In some embodiments, the GSH 5′ homology arm and the GSH 3′ homology arm bind to target sites that are spatially distinct nucleic acid sequences in the genomic safe harbor identified according to the methods as disclosed herein. In some embodiments, a nucleic acid vector composition as described herein for integration of a nucleic acid of interest into a GSH locus comprises a 5′ GSH-specific homology arm and the GSH 3′ GSH-specific homology arm that are at least 65% complementary to a target sequence in the genomic safe harbor locus identified according to the methods disclosed herein. In some embodiments, a nucleic acid vector composition as described herein for integration of a nucleic acid of interest into a GSH loci as disclosed herein comprises a 5′ GSH-specific homology arm and the 3′ GSH-specific homology arm that bind to a target site located in the PAX5 genomic safe harbor sequence, or a gene listed in Table 1A or Table 1B herein. In one embodiment the nucleic acid vector composition as described herein for integration of a nucleic acid of interest into a GSH locus does not contain any prokaryotic DNA sequence elements, for example minicircle-DNA (mcDNA), but it is contemplated that some prokaryotic-sourced DNA may be inserted as an exogenous sequence. In some embodiments, a nucleic acid vector composition as described herein for integration of a nucleic acid of interest into a GSH loci is a plasmid or a double-stranded DNA. In one aspect, a nucleic acid vector composition for integration of a nucleic acid of interest into a GSH loci as described herein includes or is obtained from a plasmid encoding in this order: a nucleotide sequence of interest (for example an expression cassette of an exogenous DNA, gene editing sequence, or donor sequence) positioned between a 5′ homology arm and a 3′ homology arm.

### (i) Nucleic Acids of Interest

In some embodiments, a nucleic acid vector composition as described herein for integration of a nucleic acid of interest into a GSH loci comprises, between the restriction cloning sites, a nucleic acid of interest. In some embodiments, the nucleic acid of interest is gene editing nucleic acid sequence as disclosed herein, and in some embodiments, the nucleic acid of interest can be for example, a heterologous gene, a nucleic acid encoding a therapeutic protein, antibody, peptide, or an antisense oligonucleic acid, or the like.

In some embodiments, the nucleic acid of interest is a RNA, e.g., RNAi, antisense nucleic acid, miRNA and variants thereof. In some embodiments, a nucleic acid of interest may comprise any sequence of interest and can also be referred to herein as an “exogenous sequence”. Exemplary nucleic acid of interests include, but are not limited to any polypeptide coding sequence (e.g., cDNAs), promoter sequences, enhancer sequences, epitope tags, marker genes, cleavage enzyme recognition sites, epitope tags and various types of expression constructs. Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate cellular metabolism resulting in enhanced cell growth rates and/or gene amplification (e.g., dihydrofolate reductase). Epitope tags are fused to a protein of interest to facilitated detection and include, for example, one or more copies of FLAG, His, myc, Tap, HA or any detectable amino acid sequence.

In some embodiments, a nucleic acid of interest can comprise one or more sequences which do not encode polypeptides but rather any type of noncoding sequence, as well as one or more control elements (e.g., promoters). In addition, a nucleic acid of interest can produce one or more RNA molecules (e.g., small hairpin RNAs (shRNAs), inhibitory RNAs (RNAis), microRNAs (miRNAs), etc.).

In some embodiments, the nucleic acid of interest encodes a receptor, toxin, a hormone, an enzyme, or a cell surface protein or a therapeutic protein, peptide or antibody or fragment thereof. In some embodiments, a nucleic acid of interest for use in the vector compositions as disclosed herein encodes any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the above. The coding sequences may be, for example, cDNAs.

In some embodiments, a nucleic acid of interest for use in the vector compositions as disclosed herein encodes a polypeptide that is lacking or non-functional in the subject having a genetic disease, including but not limited to any of the following genetic diseases listed in Table 6 in FIG. 6.

In certain embodiments, a nucleic acid of interest for use in the vector compositions as disclosed herein comprises a nucleic acid sequence that encodes a marker gene (described herein), allowing selection of cells that have undergone targeted integration, and a linked sequence encoding an additional functionality. Non-limiting examples of marker genes include GFP, drug selection marker(s) and the like.

Furthermore, although not required for expression, a nucleic acid of interest may also comprise a transcriptional or translational regulatory sequences, for example, promoters, enhancers, insulators, internal ribosome entry sites, sequences encoding 2A peptides and/or polyadenylation signals.

In some aspects, a nucleic acid of interest as defined herein encodes a nucleic acid for use in methods of preventing or treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. The method involves administration of the nucleic acid of interest (e.g., a nucleic acid as described by the disclosure) that encodes one or more therapeutic peptides, polypeptides, siRNAs, microRNAs, antisense nucleotides, etc. in a pharmaceutically-acceptable carrier to the subject in an amount and for a period of time sufficient to treat the deficiency or disorder in the subject suffering from such a disorder.

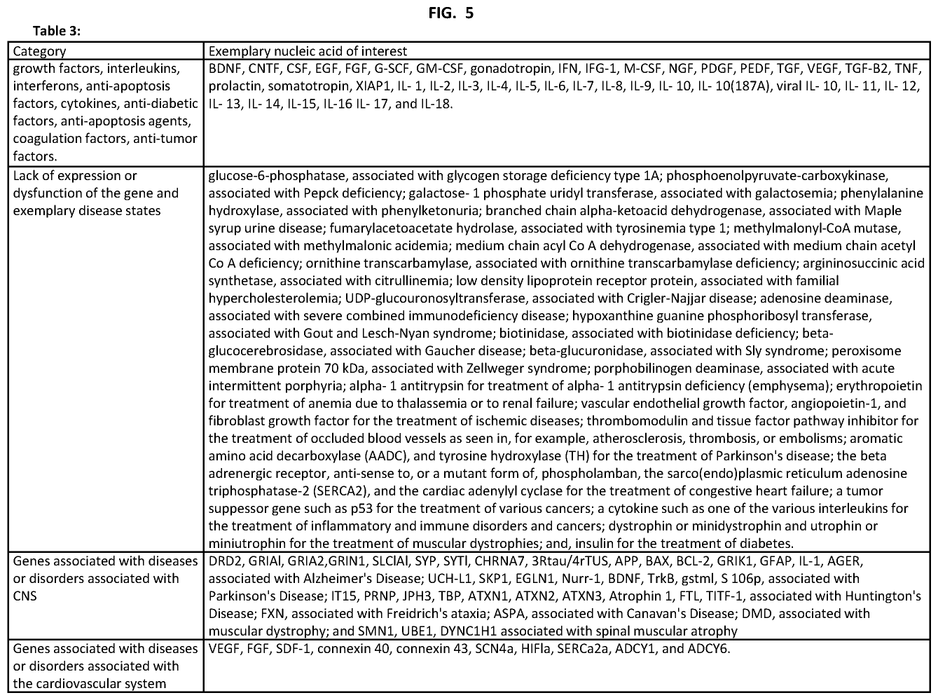

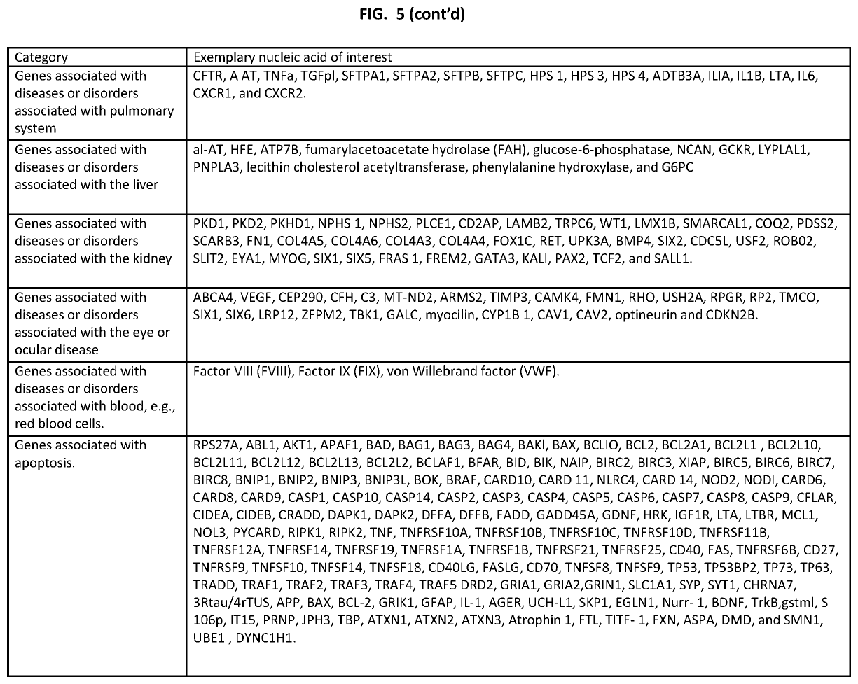

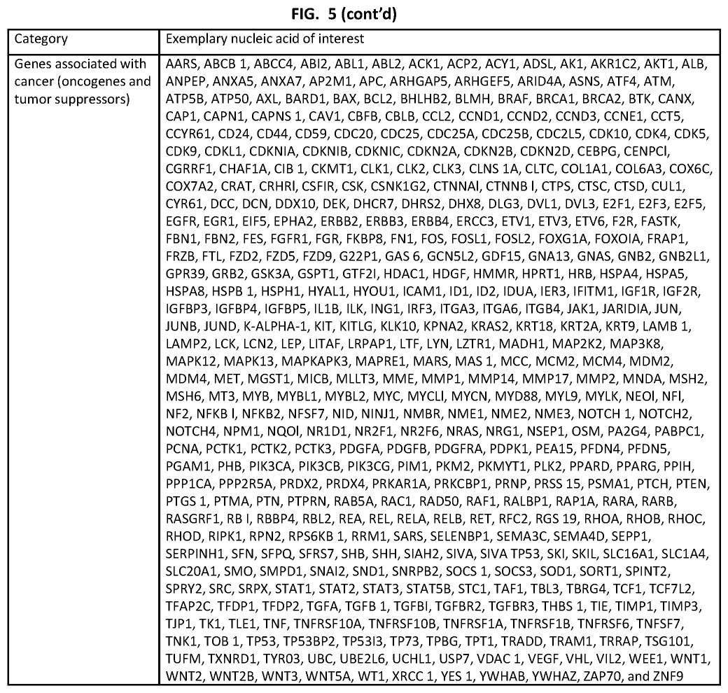

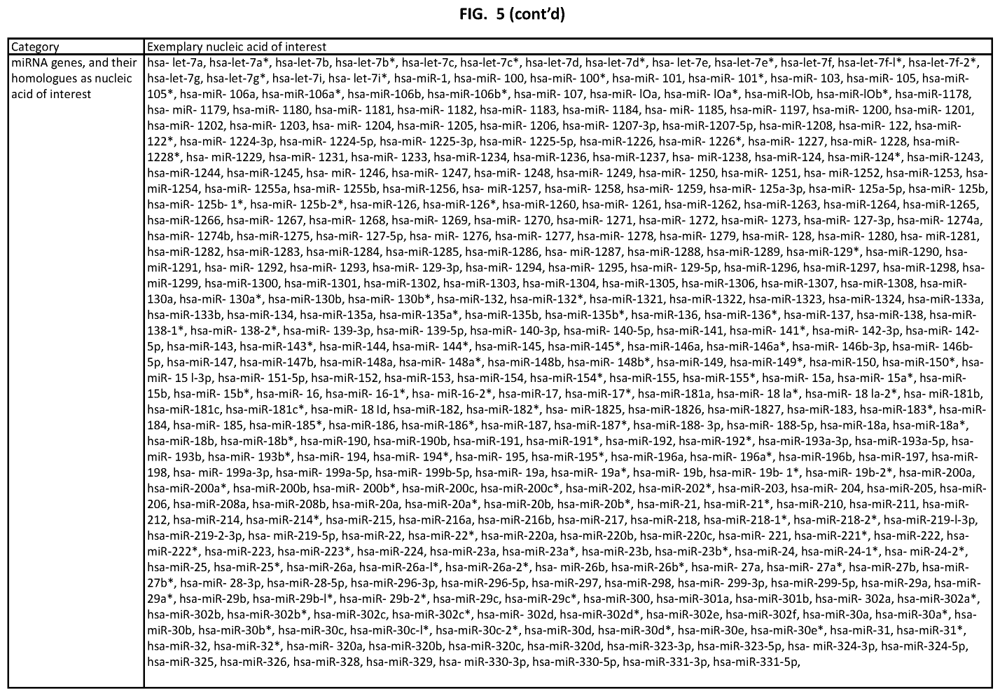

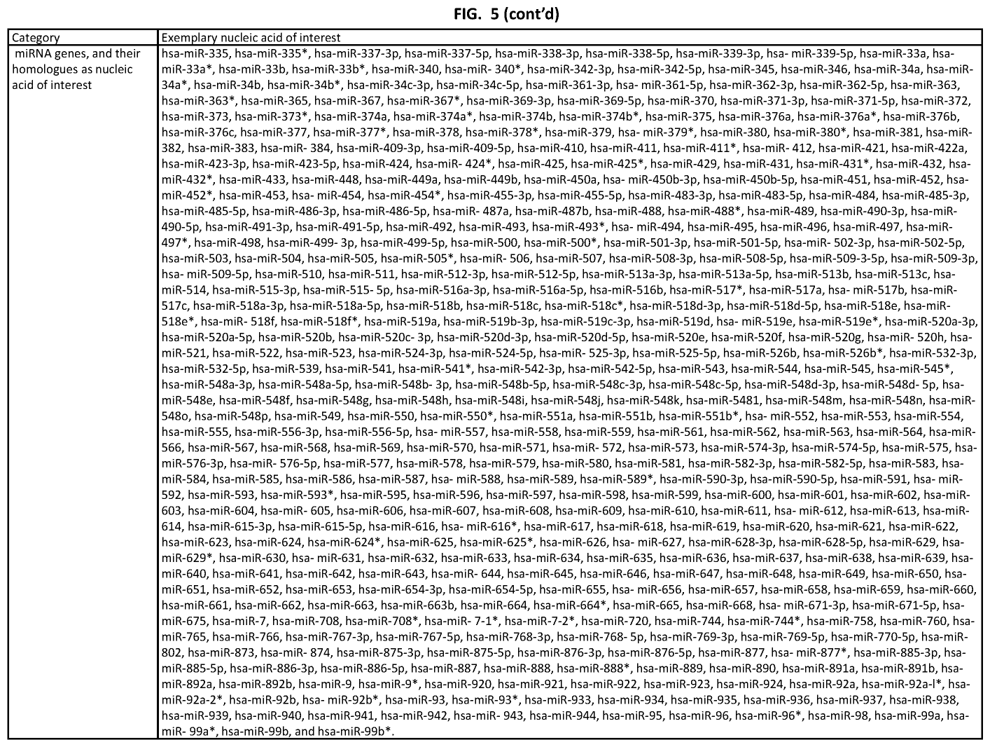

Thus in some embodiments, nucleic acids of interest for use in the vector compositions as disclosed herein can encode one or more peptides, polypeptides, or proteins, which are useful for the treatment or prevention of disease states in a mammalian subject. Exemplary nucleic acids of interest for use in the compositions and methods as disclosed herein are disclosed in the Table 3 in FIG. 5.

In some embodiments, a nucleic acid of interest for use in the vector compositions as disclosed herein can be used to restore the expression of genes that are reduced in expression, silenced, or otherwise dysfunctional in a subject (e.g., a tumor suppressor that has been silenced in a subject having cancer). A nucleic acid of interest for use in the vector compositions as disclosed herein can also be used to knockdown the expression of genes that are aberrantly expressed in a subject (e.g., an oncogene that is expressed in a subject having cancer). In some embodiments, a heterologous nucleic acid insert encoding a gene product associated with cancer (e.g., tumor suppressors) may be used to treat the cancer, by administering nucleic acid comprising the heterologous nucleic acid insert to a subject having the cancer. In some embodiments, a nucleic acid of interest as defined herein encodes a small interfering nucleic acid (e.g., shRNAs, miRNAs) that inhibits the expression of a gene product associated with cancer (e.g., oncogenes) may be used to treat the cancer. In some embodiments, a nucleic acid of interest as defined herein encodes a gene product associated with cancer (or a functional RNA that inhibits the expression of a gene associated with cancer) for use, e.g., for research purposes, e.g., to study the cancer or to identify therapeutics that treat the cancer.

A skilled artisan will also realize that the nucleic acids of interest can encode proteins or polypeptides, and that mutations that results in conservative amino acid substitutions may be made in a transgene to provide functionally equivalent variants, or homologs of a protein or polypeptide. In some aspects the disclosure embraces sequence alterations that result in conservative amino acid substitution of a transgene. In some embodiments, a nucleic acid of interest as defined herein encodes a gene having a dominant negative mutation. For example, a nucleic acid of interest as defined herein encodes a mutant protein that interacts with the same elements as a wild-type protein, and thereby blocks some aspect of the function of the wild-type protein.

In some embodiments, the nucleic acid of interest as disclosed herein also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

Table 3 in FIG. 5 discloses a non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods. A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, an small interfering nucleic acid that is substantially complementary to a miRNA is one that is capable of hybridizing with a miRNA, and blocking the miRNA's activity. In some embodiments, an small interfering nucleic acid that is substantially complementary to a miRNA is an small interfering nucleic acid that is complementary with the miRNA at all but 1, 2, 3, 4, 5, 6, 7, 8, 9, 10, 11, 12, 13, 14, 15, 16, 17, or 18 bases. In some embodiments, an small interfering nucleic acid sequence that is substantially complementary to a miRNA, is an small interfering nucleic acid sequence that is complementary with the miRNA at, at least, one base.

A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex. MicroRNA inhibitors can be expressed in cells from a transgenes of a nucleic acid, as discussed above. MicroRNA sponges specifically inhibit miRNAs through a complementary heptameric seed sequence (Ebert, M.S. Nature Methods, Epub Aug. 12, 2007). In some embodiments, an entire family of miRNAs can be silenced using a single sponge sequence. TuD RNAs achieve efficient and long-term-suppression of specific miRNAs in mammalian cells (See, e.g., Takeshi Haraguchi, et al., Nucleic Acids Research, 2009, Vol. 37, No. 6 e43, the contents of which relating to TuD RNAs are incorporated herein by reference). Other methods for silencing miRNA function (derepression of miRNA targets) in cells will be apparent to one of ordinary skill in the art.

In some embodiments, the vector as disclosed herein can further comprise, located between the restriction site, a suicide gene, operatively linked to an inducible promoter and/or tissue specific promoter. Thus, such a vector as disclosed herein can be used to kill cells upon a signal or induce cells to undergo apoptosis or programmed cell death upon a specific and discrete signal. Such a vector comprising a suicide gene can be used as an escape hatch should the gene targeting or gene editing system not function as expected.

Described herein are methods of targeted insertion of any sequence of interest into a cell. In some embodiments, a nucleic acid of interest is a nucleic acid that encodes a gene or groups of genes whose expression is known to be associated with a particular differentiation lineage of a stem cell. Sequences comprising genes involved in cell fate or other markers of stem cell differentiation can also be inserted. For example a promoterless construct containing such a gene can be inserted into a specified region (locus) such that the endogenous promoter at that locus drives expression of the gene product.

A significant number of genes and their control elements (promoters and enhancers) are known which direct the developmental and lineage-specific expression of endogenous genes. Accordingly, the selection of control element(s) and/or gene products inserted into stem cells will depend on what lineage and what stage of development is of interest. In addition, as more detail is understood on the finer mechanistic distinctions of lineage-specific expression and stem cell differentiation, it can be incorporated into the experimental protocol to fully optimize the system for the efficient isolation of a broad range of desired stem cells.

Any lineage-specific or cell fate regulatory element (e.g. promoter) or cell marker gene can be used in the compositions and methods described herein. Lineage-specific and cell fate genes or markers are well-known to those skilled in the art and can readily be selected to evaluate a particular lineage of interest. Non-limiting examples of include, but not limited to, regulatory elements obtained from genes such as Ang2, Flk1, VEGFR, MHC genes, aP2, GFAP, Otx2 (see, e.g., U.S. Pat. No. 5,639,618), Dlx (Porteus et al. (1991) Neuron 7:221-229), Nix (Price et al. (1991) Nature 351:748-751), Emx (Simeone et al. (1992) EMBO 1 11:2541-2550), Wnt (Roelink and Nuse (1991) Genes Dev. 5:381-388), En (McMahon et al.), Hox (Chisaka et al. (1991) Nature 350:473-479), acetylcholine receptor beta chain (ACHRI3) (Otl et al. (1994) J Cell. Biochem. Supplement 18A: 177). Other examples of lineage-specific genes from which regulatory elements can be obtained are available on the NCBI-GEO web site which is easily accessible via the Internet and well known to those skilled in the art.

In certain embodiments, genomic modifications (e.g., transgene integration) at a GSH locus identified herein allow integration of a nucleic acid of interest that may either utilize the promoter found at that safe harbor locus, or allow the expressional regulation of the transgene by an exogenous promoter or control element, as described herein, that is fused to the nucleic acid of interest prior to insertion. An exogenous nucleic acid of interest (i.e., in some embodiments, a target gene or transgene sequence) can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.). The exogenous nucleic acid sequence is introduced into the cell such that it is integrated into the genome of the cell at GSH loci identified according to the methods as disclosed herein, or at GSH loci listed in Table 1A or 1B.

### (ii) Nucleic Acid of Interest is a Gene Editing Gene

In some embodiments, integration of exogenous sequences can proceed through both homology-dependent and homology-independent mechanisms. Thus, the methods and vector compositions as disclosed herein can be used to insert a nucleic acid of interest or gene editing gene into a safe harbor locus identified herein, or listed in Table 1A or 1B using a CRISPR/Cas system. For example, in some embodiments, a vector composition as disclosed herein can comprise a single guide RNA comprise one or more sequences to target integration at a GSH loci identified herein, or listed in Table 1A or 1B. Non-limiting examples of single-guide RNA or guide RNA (sgRNA or gRNA) sequences suitable for targeting are shown in Table 1 in US Application 2015/0056705, which is incorporated herein in its entirety by reference.

Accordingly, in some embodiments, a nucleic acid vector composition as described herein for integration of a nucleic acid of interest into a GSH locus comprising a 3′- and 5′ GSH-specific homology arms described herein comprises at least one or more sequences for gene editing, for example, any one or more of the following: a gene editing nucleic acid sequence, a nucleic acid of interest or a guide RNA (gRNA) for a RNA-guided DNA endonuclease. In some embodiments, the gene editing nucleic acid sequence encodes a gene editing nucleic acid molecule selected from the group consisting of: a sequence specific nuclease, one or more guide RNA (gRNA), CRISPR/Cas, a ribonucleoprotein (RNP) or any combination thereof. In some embodiments, the sequence-specific nuclease comprises: a TAL-nuclease, a zinc-finger nuclease (ZFN), a meganuclease, a megaTAL, or an RNA guide endonuclease of a CRISPR/Cas sstem (e.g., Cas proteins e.g. CAS1-9, Csy, Cse, Cpf1, Cmr, Csx, Csf, cpf1, nCAS, or others). These gene editing systems are well known to those of skill in the art, See for example, TALENS described in International Patent Application No. PCT/US2013/038536, and U.S. Patent Publication No. 2017-0191078-A9 which are incorporated by reference in their entirety. CRISPR cas9 systems are known in the art and described in U.S. patent application Ser. No. 13/842,859 filed on March 2013, and U.S. Pat. Nos. 8,697,359, 8,771,945, 8,795,965, 8,865,406, 8,871,445 all of which are herein incorporated by reference in their entirety. The vectors of the present disclosure are also useful for deactivated nuclease systems, such as CRISPRi or CRISPRa dCas systems, nCas, or Cas13 systems.

In one embodiment, a nucleic acid vector composition as described herein for integration of a nucleic acid of interest into a GSH loci is provided that comprises, in the following order: a) a 5′ GSH homology arm b) a nucleic acid sequence comprising a gene editing nucleic acid directed to a GSH described herein (e.g. selected from Table 1A or Table 1B), and c) a 3′ GSH homology arm wherein the gene editing nucleic acid sequence encodes a gene editing molecule (e.g. protein or gRNA etc.) that binds to a target site located in a genomic safe harbor locus identified in the method of claim 1 or claim 11.

In some embodiments, a nucleic acid vector composition as described herein does not comprise the 3′- and 5′ GSH-specific homology arms to a GSH, but rather comprises at least one or more sequences for gene editing that target a GSH identified herein, for example, any one or more of the following sequences for gene editing: a gene editing nucleic acid sequence, a nucleic acid of interest or a guide RNA (gRNA) for a RNA-guided DNA endonuclease. Thus, in one embodiment, a nucleic acid vector composition as described herein comprises, in the following order: a portion of a GSH loci identified according to the method as disclosed herein, a guide RNA (gRNA), and a downstream portion of a GSH loci identified herein.

(iii) Guide RNAs (gRNAs)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds to a target sequence and e.g., a CRISPR associated protein that can form a ribonucleoprotein (RNP), for example, a CRISPR/Cas complex.

In some embodiments, the guide RNA (gRNA) sequence comprises a targeting sequence that directs the gRNA sequence to a desired site in the genome, is fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq.

A guide sequence can be selected to target any target sequence. In some embodiments, the target sequence is a sequence within a genome of a cell or within a GSH as disclosed herein. In some embodiments, the guide RNA can be complementary to either strand of the targeted DNA sequence. It will be appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub; Heigwer, F., et al. “E-CRISP: fast CRISPR target site identification” Nat. Methods 11, 122-123 (2014); Bae et al. “Cas-OFFinder: a fast and versatile algorithm that searches for potential off-target sites of Cas9 RNA-guided endonucleases” Bioinformatics 30(10):1473-1475 (2014); Aach et al. “CasFinder: Flexible algorithm for identifying specific Cas9 targets in genomes”BioRxiv (2014), among others).

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the single transcript further includes a transcription termination sequence, such as a polyT sequence, for example six T nucleotides. In some embodiments, a guide RNA can comprise two RNA molecules and is referred to herein as a “dual guide RNA” or “dgRNA.” In some embodiments, the dgRNA may comprise a first RNA molecule comprising a crRNA, and a second RNA molecule comprising a tracrRNA. The first and second RNA molecules may form a RNA duplex via the base pairing between the flagpole on the crRNA and the tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, the sgRNA can comprise a crRNA covalently linked to a tracrRNA. In some embodiments, the crRNA and tracrRNA can be covalently linked via a linker. In some embodiments, the sgRNA can comprise a stem-loop structure via the base-pairing between the flagpole on the crRNA and the tracrRNA. In some embodiments, a single-guide RNA is at least 50, at least 60, at least 70, at least 80, at least 90, at least 100, at least 110, at least 120 or more nucleotides in length (e.g., 75-120, 75-110, 75-100, 75-90, 75-80, 80-120, 80-110, 80-100, 80-90, 85-120, 85-110, 85-100, 85-90, 90-120, 90-110, 90-100, 100-120, 100-120 nucleotides in length). In some embodiments, a nucleic acid vector as described herein for integration of a nucleic acid of interest into a GSH loci, or composition thereof comprises a nucleic acid that encodes at least 1 gRNA. For example, the second polynucleotide sequence may encode between 1 gRNA and 50 gRNAs, or any integer between 1-50. Each of the polynucleotide sequences encoding the different gRNAs can be operably linked to a promoter. In some embodiments, the promoters that are operably linked to the different gRNAs may be the same promoter. The promoters that are operably linked to the different gRNAs may be different promoters. The promoter may be a constitutive promoter, an inducible promoter, a repressible promoter, or a regulatable promoter.

In one embodiment, a nucleic acid vector composition as described herein for integration of a nucleic acid of interest into a GSH loci encode or are administered in conjunction with another vector (e.g., an additional vector, a lentiviral vector, a viral vector, or a plasmid) that encodes a Cas nickase (nCas; e.g., Cas9 nickase or Cas9-D10A). It is contemplated herein that such an nCas enzyme is used in conjunction with a guide RNA that comprises homology to a vector as described herein and can be used, for example, to release physically constrained sequences or to provide torsional release. Releasing physically constrained sequences can, for example, “unwind” the vector such that a homology directed repair (HDR) template homology arm(s) are ex-posed for interaction with the genomic sequence. In addition, it is contemplated herein that such a system can be used to deactivate the vectors described herein, if necessary. It will be understood by one of skill in the art that a Cas enzyme that induces a double-stranded break in the vector would be a stronger deactivator of such vectors.

In one embodiment, the guide RNA comprises homology to the donor sequence or template. “Zinc finger nuclease” or “ZFN” as used interchangeably herein refers to a chimeric protein molecule comprising at least one zinc finger DNA binding domain effectively linked to at least one nuclease or part of a nuclease capable of cleaving DNA when fully assembled. “Zinc finger” as used herein refers to a protein structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

In embodiments, a nucleic acid vector composition as described herein for integration of a nucleic acid of interest into a GSH loci in accordance with the present disclosure include nucleotide sequences encoding zinc-finger recombinases (ZFR) or chimeric proteins suitable for introducing targeted modifications into the GSH identified herein. In embodiments, a nucleic acid vector composition as described herein for integration of a nucleic acid of interest into a GSH loci are suitable for use in nuclease free HDR systems such as those described in Porro et al., Promoterless gene targeting without nucleases rescues lethality of a Crigler-Najjar syndrome mouse model, EMBO Molecular Medicine, Jul. 27, 2017 (herein incorporated by reference in its entirety). In such embodiments, in vivo gene targeting approaches are suitable for the insertion of a donor sequence, without the use of nucleases. In some embodiments, the donor sequence may be promoterless.

In some embodiments, the nuclease located between the restriction sites can be a RNA-guided endonuclease. As used herein, the term “RNA-guided endonuclease” refers to an endonuclease that forms a complex with an RNA molecule that comprises a region complementary to a selected target DNA sequence, such that the RNA molecule binds to the selected sequence to direct endonuclease activity to a selected target DNA sequence in a GSH identified herein.

### (iv) CRISPR/Cas Systems

As known in the art, a CRISPR-CAS9 system includes a combination of protein and ribonucleic acid (“RNA”) that can alter the genetic sequence of an organism. CRISPR-Cas 9 provides a set of tools for Cas9-mediated genome editing via nonhomologous end joining (NHEJ) or homology-directed repair (HDR) in mammalian cells, as well as generation of modified cell lines for downstream functional studies. The CRISPR-CAS9 system continues to develop as a powerful tool to modify specific deoxyribonucleic acid (“DNA”) in the genomes of many organisms such as microbes, fungi, plants, and animals. One of ordinary skill in the art may select between a number of known CRISPR systems such as Type I, Type II, and Type III. In some embodiments, a nucleic acid vector composition as described herein for integration of a nucleic acid of interest into a GSH loci can be designed to include nucleotides encoding one or more components of these systems such as the guide sequence, tracr RNA, or Cas (e.g., Cas9). In embodiments, a single promoter drives expression of a guide sequence and tracr RNA, and a separate promoter drives Cas (e.g., Cas9) expression. One of skill in the art will appreciate that certain Cas nucleases require the presence of a protospacer adjacent motif (PAM) adjacent to a target nucleic acid sequence.

In embodiments, RNA-guided nucleases including Cas and Cas9 are suitable for use in a nucleic acid vector composition as described herein designed to provide one or more components for genome engineering using the CRISPR-Cas9 system See e.g. US publication 2014/0170753 herein incorporated by reference in its entirety.

The guide RNAs can be directed to the same strand of DNA or the complementary strand. The guide RNAs can be directed to e.g., sequences proceeding promoters, or homology domains etc.

In some embodiments, the methods and compositions described herein, e.g., a nucleic acid vector composition as described herein for integration of a nucleic acid of interest into a GSH loci can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9) that cannot generate a double strand break (DSB). This permits the endonuclease, in combination with the guide RNAs, to bind specifically to a target sequence in the genome and provide RNA-directed reversible transcriptional control.

Accordingly, in some embodiments a nucleic acid vector composition as described herein for integration of a nucleic acid of interest into a GSH loci can comprise a deactivated endonuclease, e.g., RNA-guided endonuclease and/or Cas9, wherein the deactivated endonuclease lacks endonuclease activity, but retains the ability to bind DNA in a site-specific manner, e.g., in combination with one or more guide RNAs and/or sgRNAs. In some embodiments, the vector can further comprise one or more tracrRNAs, guide RNAs, or sgRNAs. In some embodiments, the de-activated endonuclease can further comprise a transcriptional activation domain.

In embodiments, hybrid recombinases may be suitable for use a nucleic acid vector composition as described herein for integration of a nucleic acid of interest into a GSH loci to create integration cites on target DNA. For example, Hybrid recombinases based on activated catalytic domains derived from the resolvase/invertase family of serine recombinases fused to Cys2-His2 zinc-finger or TAL effector DNA-binding domains are a class of reagents capable improved targeting specificity in mammalian cells and achieve excellent rates of site-specific integration. Suitable hybrid recombinases encoded by codons in a nucleic acid vector composition as described herein for integration of a nucleic acid of interest into a GSH loci include those described in Gaj et al, Enhancing the Specificity of Recombinase-Mediated Genome Engineering through Dimer Interface Redesign, Journal of the American Chemical Society, Mar. 10, 2014 (herein incorporated by reference in its entirety).

The nucleases described herein can be altered, e.g., engineered to design sequence specific nuclease (see e.g., U.S. Pat. No. 8,021,867). Nucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

### (v) MegaTALS

In some embodiments, the endonuclease described herein can be a megaTAL. MegaTALs are engineered fusion proteins which comprise a transcription activator-like (TAL) effector domain and a meganuclease domain. MegaTALs retain the ease of target specificity engineering of TALs while reducing off-target effects and overall enzyme size and increasing activity. MegaTAL construction and use is described in more detail in, e.g., Boissel et al. 2014 Nucleic Acids Research 42(4):2591-601 and Boissel 2015 Methods Mol Biol 1239:171-196; each of which is incorporated by reference herein in its entirety. Protocols for megaTAL-mediated gene knockout and gene editing are known in the art, see, e.g., Sather et al. Science Translational Medicine 2015 7(307):ra156 and Boissel et al. 2014 Nucleic Acids Research 42(4):2591-601; each of which is incorporated by reference here-in in its entirety. MegaTALs can be used as an alternative endonuclease in any of the methods and compositions described herein.

### C. Additional Components of a Nucleic Acid Vector

In embodiments, a nucleic acid vector composition as described herein can also include a polyadenylation site upstream and proximate to the 5′ GSH-specific homology arm.

In some embodiments, a nucleic acid vector composition as described herein can comprise a Pol III promoter driven (such as U6 and H1) sgRNA expressing unit with optional orientation with respect to the transcription direction. An sgRNA target sequence for a “double mutant nickase” is optionally provided. Such embodiments increase annealing and promote HDR frequency. In some embodiments, a nucleic acid vector composition as described herein comprises, located within the restriction cloning site, a regulatory sequence operatively linked to the nucleic acid of interest, as described herein.

In embodiments, the regulatory sequence includes a suitable promoter sequence, being able to direct transcription of a gene operably linked to the promoter sequence, such as a nucleic acid of interest as that term is described herein. In embodiments, an enhancer sequence is provided upstream of the promoter to increase the efficacy of the promoter. In embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 18) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 19), and the like. In embodiments, these promoters are altered at their downstream intron containing end to include one or more nuclease cleavage sites. In embodiments, the DNA containing the nuclease cleavage site(s) is foreign to the promoter DNA.

A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as the promoters listed below. Such promoters and/or enhancers can be used for expression of any gene of interest, e.g., the gene editing molecules, donor sequence, therapeutic proteins etc.). For example, the vector may comprise a promoter that is operably linked to the DNA endonuclease or CRISPR/Cas9-based system. The promoter operably linked to the CRISPR/Cas9-based system or the site-specific nuclease coding sequence may be a promoter from simian virus 40 (SV40), a CAG promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a liver specific promoter, natural or synthetic. In one embodiment, delivery to the liver can be achieved using endogenous ApoE specific targeting of the composition comprising a vector to hepatocytes via the low density lipoprotein (LDL) receptor present on the surface of the hepatocyte.

### D. Vectors

Vectors disclosed herein, e.g., a nucleic acid vector comprising a portion of a GSH, or a nucleic acid vector composition comprising at GSH-5′ homology arm, and a 3′GSH homology arm flanking a nucleic acid comprising a restriction cloning site for integrating the flanked nucleic acid into the genome at a GSH by homologous recombination, as described herein, can be a viral vector or a non-viral vector. Viral vectors and non-viral vectors are well known in the art.

Any vector systems may be used including, but not limited to, plasmid vectors, retroviral vectors, lentiviral vectors, adenovirus vectors, poxvirus vectors; herpesvirus (HSV) vectors and adeno-associated virus vectors, vaccinia virus vectors, bacteriophage vectors etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleic acids of interests are introduced into the cell, if the nucleic acid of interest is a gene editing nucleic acid of interest, additional nucleases and/or donor sequences may be carried on the same vector or on different vectors. When multiple vectors are used, each vector may comprise one or more nucleic acid of interest as described herein.

A. Non-Viral Vectors:

Examples of non-viral vectors for use can transform prokaryotic or eukaryotic cells and be replication and/or expression. Vectors can be prokaryotic vectors, e.g., plasmids, or shuttle vectors, insect vectors, or eukaryotic vectors. Expression vectors can also be for administration to a plant cell, animal cell, preferably a mammalian cell or a human cell, fungal cell, bacterial cell, or protozoal cell using standard techniques described for example in Sambrook et al., supra and United States Patent Publications 20030232410; 20050208489; 20050026157; 20050064474; and 20060188987, and International Publication WO 2007/014275.

Other non-viral vectors encompassed for use as a nucleic acid composition as described herein include, for example, DNA plasmids, naked nucleic acid, naked phage DNA, minicircle DNA, and linear plasmids (e.g., disclosed in US2009/0263900) and nucleic acid complexed with a delivery vehicle such as a liposome or poloxamer. Circular DNA expression vectors or minicircle vectors are disclosed in WO2002/083889, WO2014/170,238, WO2004/099420, WO20102/026099, U.S. Pat. Nos. 6,143,530, 5,622,866, 7,622,252, 8,460,924, 6,277,608, US application 2003/0032092, 2004/0214329, which are incorporated herein in their entirety by reference.

Vectors suitable in the methods and compositions as disclosed herein include linear covalently closed DNA vectors, such as those described in Nafiseh, and Roderick Slavcev. “Construction and characterization of an in-vivo linear covalently closed DNA vector production system.” Microbial cell factories 11.1 (2012): 154, as well as linear covalently closed (LCC) mini-plasmids (Slavcev, Roderick, Chi Hong Sum, and Nafiseh Nafissi. “Optimized production of a safe and efficient gene therapeutic vaccine versus HIV via a linear covalently closed DNA minivector.” BMC Infectious Diseases 14.S2 (2014): P74), or DNA ministrings (described in U.S. Pat. No. 9,290,778 and Nafiseh, et al. “DNA ministrings: highly safe and effective gene delivery vectors.” Molecular Therapy—Nucleic Acids 3.6 (2014): e165; Wong, Shirley, et al. “Production of double-stranded DNA ministrings.” Journal of visualized experiments: JoVE 108 (2016)) or ceDNA vectors (Li L, et al, (2013) Production and Characterization of Novel Recombinant Adeno-Associated Virus Replicative-Form Genomes: A Eukaryotic Source of DNA for Gene Transfer. PLoS ONE 8(8): e69879).

Non-viral vectors encompassed for use in the methods and compositions as disclosed herein include, for example, minimized vectors, plasmids (including antibiotic free plamids), miniplasmids, minicircle, minivectors, such as those described in Hardee, Cinnamon L., et al. “Advances in non-viral DNA vectors for gene therapy.” Genes 8.2 (2017): 65. Examples of circular covalently closed vectors (CCC vectors) include minicircles, minivectors and miniknots. Examples of linear covalently closed (LCC) vectors include MIDGE, MiLV, ministring. Mini-intronic plasmids can also be used. These are described in Table 2 in Hardee, Cinnamon L., et al. “Advances in non-viral DNA vectors for gene therapy.” Genes 8.2 (2017): 65.

Non-viral vectors encompassed for use in the methods and compositions as disclosed herein include, for example, plasmids DNA vectors (pDNA expression vectors), as discussed in review article Gill, et al., “Progress and prospects: the design and production of plasmid vectors.” Gene therapy 16.2 (2009): 165-171, and Yin, Hao, et al. “Non-viral vectors for gene-based therapy.” Nature Reviews Genetics 15.8 (2014): 541-555.

Viral vectors include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11: 167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

B. Viral Vectors:

A viral vector refers to a virus or viral chromosomal material into which a fragment of foreign DNA can be inserted for transfer into a cell. Any virus that includes a DNA stage in its life cycle may be used as a viral vector in the subject methods and compositions. For example, the virus may be a single strand DNA (ssDNA) virus or a double strand DNA (dsDNA) virus. Also suitable are RNA viruses that have a DNA stage in their lifecycle, for example, retroviruses, e.g. MMLV, lentivirus, which are reverse-transcribed into DNA. The virus can be an integrating virus or a non-integrating virus.

Viral vectors encompassed for use in the methods and compositions as disclosed herein are discussed in review article Hendrie, Paul C., and David W. Russell. “Gene targeting with viral vectors.” Molecular Therapy 12.1 (2005): 9-17 and Perez-Pinera, “Advances in targeted genome editing.” Current opinion in chemical biology 16.3 (2012): 268-277.

Adeno-associated virus (“AAV”) vectors are encompassed for use as nucleic acid vector compositions as disclosed herein, and are useful for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). At least six viral vector approaches are currently available for gene transfer in clinical trials, which utilize approaches that involve complementation of defective vectors by genes inserted into helper cell lines to generate the transducing agent.

As one non-limiting example, one virus of interest is adeno-associated virus. By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

By a “recombinant AAV vector”, or “rAAV vector” it is meant an AAV virus or AAV viral chromosomal material comprising a polynucleotide sequence not of AAV origin (i.e., a polynucleotide heterologous to AAV), typically a nucleic acid sequence of interest to be integrated into the cell following the subject methods. In general, the heterologous polynucleotide is flanked by at least one, and generally by two AAV inverted terminal repeat sequences (ITRs). In some instances, the recombinant viral vector also comprises viral genes important for the packaging of the recombinant viral vector material. By “packaging” it is meant a series of intracellular events that result in the assembly and encapsidation of a viral particle, e.g. an AAV viral particle. Examples of nucleic acid sequences important for AAV packaging (i.e., “packaging genes”) include the AAV “rep” and “cap” genes, which encode for replication and encapsidation proteins of adeno-associated virus, respectively. The term rAAV vector encompasses both rAAV vector particles and rAAV vector plasmids.

A “viral particle” refers to a single unit of virus comprising a capsid encapsidating a virus-based polynucleotide, e.g. the viral genome (as in a wild type virus), or, e.g., the subject targeting vector (as in a recombinant virus). An “AAV viral particle” refers to a viral particle composed of at least one AAV capsid protein (typically by all of the capsid proteins of a wild-type AAV) and an encapsidated polynucleotide AAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

Recombinant adeno-associated virus (“rAAV”) vectors are encompassed for use as nucleic acid vector compositions as disclosed herein All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV2, AAV3, AAV4, AAV5, AAV6, AAV7, AAV8, AAV9 and AAVrh. 10 and any novel AAV serotype can also be used in accordance with the present invention.

Replication-deficient recombinant adenoviral vectors (Ad) are also encompassed for use herein, can be produced at high titer and readily infect a number of different cell types. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Retroviral vectors are encompassed for use as nucleic acid vector compositions as disclosed herein. pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)).

Vectors suitable in the methods and compositions as disclosed herein include lentivirus vectors, such as those disclosed in Picanço-Castro, “Advances in lentiviral vectors: a patent review.” Recent patents on DNA & gene sequences 6.2 (2012): 82-90. The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats (LTRs) with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al, J. Virol. 66:1635-1640 (1992); Sommerfelt et al, Virol. 176:58-59′ (1990); Wilson et al, J. Virol. 63:2374-2378 (1989); Miller et al, J. Virol. 65:2220-2224 (1991); PCT/US94/05700). Other retroviral vectors for use herein include foamy viruses, as disclosed in Sweeney, Nathan Paul, et al. “Delivery of large transgene cassettes by foamy virus vector.” Scientific reports 7 (2017): 8085.

Lentiviral transfer vectors can be produced generally by methods well known in the art. See, e.g., U.S. Pat. Nos. 5,994,136; 6,165,782; and 6,428,953, US application 2014/0315294 and described in Merten et al “Production of lentiviral vectors.” Molecular Therapy-Methods & Clinical Development 3 (2016): 16017 and Merten, et al. “Large-scale manufacture and characterization of a lentiviral vector produced for clinical ex vivo gene therapy application.” Human gene therapy 22.3 (2010): 343-356, each of which are incorporated herein in their entirety by reference. In some embodiments, the lentivirus is an integrase deficient lentiviral vector (IDLV). IDLVs may be produced as described, for example using lentivirus vectors that include one or more mutations in the native lentivirus integrase gene, for instance as disclosed in Leavitt et al. (1996) J. Virol. 70(2):721-728; Philippe et al. (2006) Proc. Nat 1I Acad. ScL USA 103(47): 17684-17689; and WO 06/010834. Lentiviruses for use in the methods and compositions as disclosed herein are disclosed in U.S. Pat. Nos. 6,207,455, 5,994,136, 7,250,299, 6,235,522, 6,312,682, 6,485,965, 5,817,491; 5,591,624,

Vectors suitable in the methods and compositions as disclosed herein include non-integrating lentivirus vectors (IDLV). See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222; U.S. Patent Publication No 2009/054985. In certain embodiments, the IDLV is an HIV lentiviral vector comprising a mutation at position 64 of the integrase protein (D64V), as described in Leavitt et al. (1996) J. Virol. 70(2):721-728. Additional IDLV vectors suitable for use herein are described in U.S. patent application Ser. No. 12/288,847, incorporated by reference herein.

Vectors suitable in the methods and compositions as disclosed herein include recombinant HCMV and RHCMV vectors, as disclosed in US 2013/0136,768.

Nucleic acid vectors useful herein for introduction of a nucleic acid of interest into a hematopoietic stem cell, e.g., CD34+ cells, include adenovirus Type 35. Nucleic acid vectors useful herein for introduction of a nucleic acid of interest into immune cells (e.g., T-cells) include non-integrating lentivirus vectors. See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

Vectors suitable in the methods and compositions as disclosed herein include baclulovirus expression vector systems (BEVS), which are discussed in Felberbaum. “The baculovirus expression vector system: a commercial manufacturing platform for viral vaccines and gene therapy vectors.” Biotechnology journal 10.5 (2015): 702-714.

Vectors suitable in the methods and compositions as disclosed herein include the HSV Type 1 (HSV-1)-AAV hybrid vectors, for example, as disclosed in Heister, Thomas, et al. “Herpes simplex virus type 1/adeno-associated virus hybrid vectors mediate site-specific integration at the adeno-associated virus preintegration site, AAV51, on human chromosome 19.” Journal of virology 76.14 (2002): 7163-7173, and 5,965,441. Other hybrid vectors can be used, e.g., disclosed in U.S. Pat. No. 6,218,186.

### IV. Kits

Another aspect of the technology described herein relates to kits, e.g., kits for insertion of a gene or nucleic acid sequence into a target GSH identified according to the methods as disclosed herein, as well as primer sets to determine integration of the gene or nucleic acid sequence.

In some embodiment, the kit comprises: (a) a vector composition as described herein, and primer pairs to determine integration by homologous recombination of nucleic acid located between the restriction site located between the 3′ GSH-specific homology arm and the 5′ GSH-specific homology arm of the vector. In some embodiments, the kit comprises primer pairs that span the site of integration, where the primer pair comprises at least a GSH 5′ primer and at least one GSH 3′ primer, wherein the GSH is identified according to the methods as disclosed herein, wherein the at least one GSH 5′ primer binds to a region of the GSH upstream of the site of integration, and the at least one GSH 3′ primer is at least binds to a region of the GSH downstream of the site of integration. Such primer pairs can function to act as a negative control and do produce a short PCR product when no integration has occurred, and produce no, or a long PCR product incorporating the inserted nucleic acid when nucleic acid insertion has occurred.

In some embodiments, the kit can comprise (a) a GSH-specific single guide and an RNA guided nucleic acid sequence comprised in one or more GSH vectors; and (b) GSH knock-in vector comprising GSH vector wherein one or more of the sequences of (a) or (b) are comprised on a vector as described herein. In some embodiments, the GSH vector is a GSH-CRISPR-Cas vector or other GSH-gene editing vector as comprising a gene editing gene as described herein. In some embodiments, the GSH CRISPR-Cas vector comprises a GSH-sgRNA nucleic acid sequence and Cas9 nucleic acid sequence.

In another embodiment, the kit can further comprise a GSH knockin donor vector comprising a GSH 5′ homology arm and a GSH 3′ homology arm, wherein the GSH 5′ homology arm and the GSH 3′ homology arm are at least 65% complementary to a sequence in the genomic safe harbor (GSH) identified according to the methods as disclosed herein, and where the GSH 5′ and 3′ homology arms allow (i.e., guide) insertion, by homologous recombination, of the nucleic acid sequence located between the GSH 5′ homology arm and a GSH 3′ homology arm into a loci located within the genomic safe harbor. In some embodiments, the GSH Cas9 knockin donor vector is a PAX5 Cas9 knockin donor vector comprising a PAX5 5′ homology arm and a PAX5 3′ homology arm, wherein the PAX5 5′ homology arm and the PAX5 3′ homology arm are at least 65% complementary to the PAX5 genomic safe harbor loci, and wherein the PAX5 5′ and 3′ homology arms guide insertion, by homologous recombination, of the nucleic acid located between the GSH 5′ homology arm and a GSH 3′ homology arm into a loci within the PAX5 genomic safe harbor.

In some embodiments, the kit comprises a GSH vector which is GSH Cas9 knock in donor vector.

In some embodiments, the kit further comprising at least one GSH 5′ primer and at least one GSH 3′ primer, wherein the at least one GSH 5′ primer is at least 80% complementary to a region of the GSH upstream of the site of integration, and the at least one GSH 3′ primer is at least 80% complementary to a region of the GSH downstream of the site of integration.

In some embodiments, the kit can comprise two primer pairs, each primer pair functioning as a positive control. For example, in some embodiments, the kit comprises (a) at least two GSH 5′ primers comprising a forward GSH 5′ primer that binds to a region of the GSH upstream of the site of integration, and a reverse GSH 5′ primer that binds to a sequence in the nucleic acid inserted at the site of integration in the GSH sequence, and (b) at least two GSH 3′ primers comprising a forward GSH 3′ primer that binds to a sequence located at the 3′ end of the nucleic acid inserted at the site of integration in the GSH sequence, and a reverse GSH 3′ primer binds to a region of the GSH downstream of the site of integration. In such an embodiment, the primer pairs can function to act as a positive and produce a PCR product only when integration has occurred, and no PCT product is produced when integration has not occurred.

In some embodiments, the kit can comprise at least two GSH 5′ primers comprising;

a forward GSH 5′ primer that is at least 80% complementary to a region of the GSH u-stream of the site of integration, and a reverse GSH 5′ primer that is at least 80% complementary to a sequence in the nucleic acid inserted at the site of integration in the GSH sequence.

In some embodiments, the kit can further comprise at least two GSH 3′ primers comprising; a forward GSH 3′ primer that is at least 80% complementary to a sequence located at the 3′ end of the nucleic acid inserted at the site of integration in the GSH sequence, and a reverse GSH 3′ primer that is at least 80% complementary to a region of the GSH down-stream of the site of integration.

In some embodiments, the kits as disclosed herein can comprise a GSH 5′ primer which is a PAX5 5′ primer and a GSH 3′ primer which is a PAX 3′ primer, wherein the PAX5 5′ primer and the PAX5 3′ primer flank the site of integration in the PAX5 genomic safe harbor.

### V. Transgenic Mice

Another aspect of the technology described herein relates to a transgenic animal, such as a transgenic mouse strain generated with a nucleic acid of interest inserted into a GSH identified according to the methods as disclosed herein.

In some embodiments, one aspect of the invention relates to a transgenic mouse comprising a nucleic acid of interest, such as but not limited to, a nucleic acid encoding a marker gene, therapeutic protein or inserted into the genomic DNA of the mouse at a GSH loci identified according to the methods disclosed herein, where the reporter gene is flanked by lox sites, e.g., LoxP sites. In some embodiments, the GSH loci is located in the genomic DNA of the host animal, e.g., mouse in any of the genes selected from Table 1A or Table 1B. In some embodiments, the GSH loci is located in the intronic or intragenic or untranslated region (e.g., 3′UTR, 5′UTR exonic) nucleic acid sequence of the PAX5 gene.

Another aspect of the invention as disclosed herein relates to a method of generating a genetically modified animal, such as, e.g., a transgenic mouse, comprising a nucleic acid interest inserted at a Genomic Safe Harbor (GSH) identified according to the methods disclosed herein, where the method comprises a) introducing into a host cell a vector as disclosed herein, and b) introducing the cell into a carrier animal to produce a genetically modified animal. In some embodiments, the host cell is a zygote or a pluripotent stem cell.

Another aspect relates to a genetically modified animal produced by the methods disclosed herein.

VI. Delivery of Nucleic acid Vectors

Various techniques and methods are known in the art for delivering nucleic acids to cells, and are encompassed for use in the delivery of the nucleic acid vectors described herein, including non-viral vectors comprising a portion of the GSH or nucleic acid vectors comprising 5′- and 3′GSH-specific homology arms. For example, nucleic acids can be formulated into lipid nanoparticles (LNPs), lipidoids, liposomes, lipid nanoparticles, lipoplexes, or core-shell nanoparticles. Typically, LNPs are composed of nucleic acid molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol). Exemplary lipid nanoparticles and methods for preparing the same are described, for example, in WO2015/074085, WO2016081029, WO2015/199952, WO2017/117528, WO2017/075531, WO2017/004143, WO2012/040184, WO2012/061259, WO2011/149733, WO2013/158579, WO2014/130607, WO2011/022460, WO2013/148541, WO2013/116126, WO2011/153120, WO2012/044638, WO2012/054365, WO2008/042973, WO2010/129709, WO2010/144740, WO2012/099755, WO2013/049328, WO2013/086322, WO2013/086354, WO2013/086373, WO2014/008334, WO2011/075656, WO2011/071860, WO2009/132131, WO2010/088537, WO2010/054401, WO2010/054384, WO2010/054406, WO2010/054405, WO2010/048536, WO2009/082607, WO2014/0144740, WO2012/016184, WO2014/152211, WO2017/049074, WO1996/040964, WO1999/018933, WO2009/086558, WO2010/129687, WO2010/147992 WO2010/042877, WO2009/108235, WO2014/081887, WO2005/120461, WO2011/000106, WO2011/000107, WO2015/011633, WO2005/120152, WO2011/141705, WO2016/197133, WO2015/011633, WO2013/126803, WO2012/000104, WO2011/141705, WO2006/007712, WO2011/038160, WO2005/121348, WO2005/120152, WO2011/066651, WO2009/127060, WO2011/141704, WO2006/074546, WO2005/121348, WO2006/069782, WO2009027337, WO2012030901, WO2012031043, WO2012/031046, WO2013/006825, WO2013/033563, WO2013/040429, WO2014/043544, WO2016/130963, WO2017/181026, and WO2013/089151, contents of all of which is incorporated herein by reference in their entireties. In some embodiments, the lipid nanoparticle, in addition to the nucleic acid, comprises lipids in the following molar ratio: 50% cationic lipid, 10% non-ionic lipid (e.g., phospholipid, such as distearoylphosphatidylcholine (DSPC)), 38.5% cholesterol and 1.5% PEG-lipid (e.g., 242-(w-methoxy(polyethyleneglyco12000)ethoxy 1-N,N-ditetradecylacetamide (PEG2000-DMA)).

Another method for delivering nucleic acids to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515, WO2017/177326 contents of all of which is incorporated herein by reference in their entirety.

Nucleic acids can also be delivered to a cell by electroporation. Generally, electroporation uses pulsed electric current to increase the permeability of cells, thereby allowing the nucleic acid to move across the plasma membrane. Electroporation techniques are well known in the art and are used to deliver nucleic acids in vivo and clinically. See, for example, Andre et al., Curr Gene Ther. 2010 10:267-280; Chiarella et al, Curr Gene Ther. 2010 10:281-286; Hojman, Curr Gene Ther. 2010 10: 128-138; contents of all of which are herein incorporated by reference in their entirety. Electroporation devices are sold by many companies worldwide including, but not limited to BTX® Instruments (Holliston, Mass.) (e.g., the AgilePulse In Vivo System) and Inovio (Blue Bell, Pa.) (e.g., Inovio SP-5P intramuscular delivery device or the CELLECTRA® 3000 intradermal delivery device). Electroporation can be used after, before and/or during administration of the nucleic acid vector. Additional exemplary methods and apparatus for delivering nucleic acids utilizing electroporation are described, for example, in U.S. Pat. Nos. 5,273,525, 6,520,950, 6,654,636 and 6,972,013, contents of all of which are incorporated herein by reference in their entirety.

Nucleic acids can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™(Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT4™ (Dharmacon), ESCORT™ III (Sigma, St. Louis, Mo.), and ESCORT™ IV (Sigma Chemical Co.). Nucleic acids, can also be delivered to a cell via microfluidics methods known to those of skill in the art.

Methods of non-viral delivery of nucleic acids in vivo or ex vivo include electroporation, lipofection (see, U.S. Pat. Nos. 5,049,386; 4,946,787 and commercially available reagents such as Transfectam™ and Lipofectin™), microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid:nucleic acid conjugates, naked DNA, artificial virions, viral vector systems (e.g., retroviral, lentivirus, adenoviral, adeno-associated, vaccinia and herpes simplex virus vectors as described in WO 2007/014275) and agent-enhanced uptake of DNA. Sonoporation using, e.g., the Sonitron 2000 system (Rich-Mar) can also be used for delivery of nucleic acids.

Vectors (e.g., retroviruses, adenoviruses, liposomes, etc.) comprising nucleic acids as described herein can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector composition as disclosed herein into hematopoietic stem cells are disclosed, for example, in U.S. Pat. No. 5,928,638.

The nucleic acid vector compositions as disclosed herein can be used for ex vivo cell transfection for diagnostics, research, or for gene therapy (e.g., via re-infusion of the transfected cells into the host organism). In some embodiments, cells are isolated from the subject organism, transfected with a nucleic acid vector a composition as disclosed herein, and re-infused back into the subject organism (e.g., patient or subject). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panb cells), GR-1 (granulocytes), and lad (differentiated antigen presenting cells) (see Inaba et al., J. Exp. Med. 176:1693-1702 (1992)). In one embodiment, the cell to be used is an oocyte. In other embodiments, cells derived from model organisms may be used. These can include cells derived from xenopus, insect cells (e.g., drosophilia) and nematode cells.

### VII. Use of GSH in Modified AAV

Current AAV-based therapeutic approaches to disease treatment contend with the fundamental challenge that mammalian immune systems detect, recognize and eliminate virus from the individual's system. In some cases, a patient may already have been naturally exposed to the same strain of AAV that forms the basis for the therapeutic, and so the viral-based therapeutic is cleared from the patient before it can have therapeutic effect. Expanding the diversity of recombinant AAV capsids may not only avoid this immune surveillance problem, but additionally may optimize the biodistribution of the viral therapeutic.

Recombinant dependoparvoviral vectors can be produced which use the capsid of one virus and the rest of the genome of another. Since each virus essentially undergoes a purifying selection during each infectious cycle in nature, each viral strain is continuously maintained in a state of “fitness” for its specific biological niche, and genetic engineering has exploited these differences to make a set of modified AAV for therapeutic purposes. However, the relatively limited number of strains also limits the number of these engineered vectors, and the likelihood of prior immune system sensitization to them remains significant. Previous efforts to generate less recognizable recombinant AAV-based vectors with desired properties have largely focused on completely artificial criteria unrelated to actual viral survival. One common approach has been to engineer rAAV vectors through the use of combinatorial libraries that introduce in most cases limited random codons into the vector-encoding nucleotides. The resulting vectors are then screened and selected from desirable phenotype(s) in vivo and/or in vitro. Another approach is capsid “shuffling”, in which fragmented capsid open reading frames (“ORFs”) from closely related AAV species are recombined and reassembled into full-length capsid ORFs with a correspondingly novel arrangement of motifs. A third approach uses rational capsid design to modify discrete capsid surface motifs and so tailor the phenotype in a controlled manner.

But all of these approaches rely on a fundamentally limited set of modern-day capsids from the currently known set of AAV. The invention affords an improved solution to the creation of novel capsids for rAAV vectors: the GSH sequences of the invention (essentially heritable dependoparvovirus capsid sequences) may be used in the construction of variant viral capsids. EVEs represent an infection of an individual animal of that species at least one generation prior to the current one, and if phyletic inheritance is seen, then the EVE was acquired pre-speciation. Thus, EVEs are the vestiges of ancient dependoparvovirus species that have either evolved into the modern circulating dependoparvovirus species or have become extinct in the intervening time. Further, they are host species co-adapted. Since rAAV and viral fitness are independently selected, then these ancestral dependoparvovirus capsids may contain evolutionarily “discarded” motifs that (i) are unlikely to have been previously seen by a potential patient's immune system, and (ii) may provide useful attributes to gene therapy vectors.

The GSH sequences and EVEs identified herein may be utilized as short linear sequences inserted into the surface-exposed region (e.g., a variable region) of a dependoparvovirus capsid. The variable region of the dependoparvovirus capsid may be selected from the capsid variable region of AAV I, II, III, IV, V, VI, VII, VIII, or IX. In another version of the approach, a GSH sequence or EVE sequence of the invention is used as a short linear sequence inserted into a tertiary structural element of a dependoparvovirus. The tertiary structural element can be a 3-fold axis of symmetry. Alternatively, the entire capsid may be reconstituted using the inferred or consensus Cap sequences from orthologous species. The icosahedral Ti symmetry AAV capsids are assembled from 60 subunits (VP1:VP2:VP3; 1:1:10 approximate ratio) with a conserved beta-barrel core composed of the anti-parallel βBDIG and βCHEF sheets. The VR, HI- and D-loops together with the capsid variable regions described above constitute the regions of greatest diversity among the capsids and may provide a convenient locus for modification with the GSH sequences and EVEs of the invention.

Some embodiments of the technology described herein can be defined according to any of the following numbered paragraphs:
1. A method to identify genomic safe harbor (GSH) regions in a mammalian genome, comprising;
   a. identifying the loci of the endogenous virus element (EVE) of the genome of ur-species or in related species within taxonomic rank order;
   b. identifying the interspecific conserved loci in the human or mouse genome;
   c. validating the loci as a genomic safe harbors in human or mouse germlines using at least one in vitro or in vivo assays selected from any one or more of:
      i. insertion of a marker gene into the loci in human cells and measure marker gene expression in vitro;
      ii. insertion of marker gene into orthologous loci in progenitor cells or stem cells and engraft the cells into immune-depleted mice and/or assess marker gene expression in all developmental lineages;
      iii. differentiate hematopoietic CD34+ cells into terminally differentiated cell types, wherein the hematopoietic CD34+ cells have a marker gene inserted into the loci identified in step b; or
      iv. generate transgenic knock-in mouse wherein the genomic DNA of the mouse has a marker gene inserted in the loci identified in step b, wherein the marker gene is operatively linked to a tissue specific or inducible promoter.
2. The method of paragraph 1, wherein the GSH is intragenic or intergenic.
3. The method of paragraph 1, wherein the EVE is a nucleic acid sequence encoding intronic or exonic viral nucleic acid, viral DNA or DNA copies of viral RNA.
4. The method of paragraph 3, wherein the viral nucleic acid is non-retroviral nucleic acid or non-retroviral provirus.
5. The method of paragraph 4, wherein the non-retroviral nucleic acid is from a parvovirus or circovirus.
6. The method of paragraph 5, wherein the parvovirus is selected from group consisting of B19, minute virus of mice (mvm), RA-1, AAV, bufavirus, hokovirus, bocovirus, or any of the parvoviruses listing in Table 2 or Table 4A or Table 4B.
7. The method of paragraph 6, wherein the parvovirus is AAV.
8. The method of paragraph 5, wherein the circovirus is porcrine circovirus (PCV) (e.g., PCV-1, PCV-2).
9. The method of paragraph 4, wherein the non-retroviral nucleic acid encodes non-structural and/or structural viral proteins, e.g., rep (replication) and/or cap (capsid) proteins.
10. The method of paragraph 1, wherein the ur-species are selected from any of the group of: Cetacea, Chiropetera, Lagomorpha, Macropodiadae.
11. A method to identify genomic safe harbor (GSH) regions in a mammalian genome, comprising;
a) performing comparative genomic approaches to:
   i) compare the interspecific introns of collinearly organized and/or synteny organized genes between species to identify an enlarged intron in one species relative to another species, and/or
   ii) compare intergenic distance (or space) between adjacent genes or selected genes that are collinearly organized or synteny organized between species to identify a large variation in the intergenic distance (or space);
b) selecting the enlarged intron in step a(i) or intergenic space between selected genes in step a(ii) as a loci for a genomic safe harbor;
c) validating the loci as a genomic safe harbor in human or mouse germlines using at least one in vitro or in vivo assays selected from any one or more of:
   i. insertion of a marker gene into the loci in human cells and measure marker gene expression in vitro;
   ii. insertion of marker gene into orthologous loci in progenitor cells or stem cells and engraft the cells into immune-depleted mice and/or assess marker gene expression in all developmental lineages;
   iii. differentiate hematopoietic CD34+ cells into terminally differentiated cell types, wherein the hematopoietic CD34+ cells have a marker gene inserted into the loci identified in step b; or
   iv. generate transgenic knock-in mouse wherein the genomic DNA of the mouse has a marker gene inserted in the loci identified in step b, wherein the marker gene is operatively linked to a tissue specific or inducible promoter.
12. A nucleic acid vector comprising at least a portion of the genomic safe harbor (GSH) nucleic acid identified as a genomic safe harbor in the method of any of paragraphs 1 to 11.
13. The nucleic acid vector of paragraph 12, wherein the vector is a viral vector or a non-viral vector.
14. The nucleic acid of paragraph 12, wherein the at least a portion of the GSH nucleic acid comprises the PAX5 genomic DNA or a fragment thereof
15. The nucleic acid vector of paragraph 12, wherein the GSH nucleic acid comprises an untranslated sequence or an intron of the PAX5 gene.
16. The nucleic acid of paragraph 12, wherein the at least a portion of the GSH nucleic acid comprises the Kif5 genomic DNA or a fragment thereof
17. The nucleic acid vector of paragraph 12, wherein the GSH nucleic acid comprises an untranslated sequence or an intron of the Kif5 gene.
18. The nucleic acid vector of paragraph 12, wherein the GSH nucleic acid is a nucleic acid selected from any of the nucleic acid sequences listed in Table 1A or Table 1B.
19. The nucleic acid vector of paragraph 12, wherein the at least portion of the GSH comprises at least one modification as compared to the wild-type GSH sequence.
20. The nucleic acid vector of paragraph 19, wherein the modification is a nucleic acid sequence comprising a restriction cloning site.
21. The nucleic acid vector of paragraph 19, wherein the modification is a nucleic acid sequence comprising one or more target sites for one or more nucleases.
22. The nucleic acid vector of paragraph 21, wherein the nuclease is selected from a zinc finger nuclease (ZFN), a TAL-effector domain nuclease (TALEN), or a CRISPR/Cas system.
23. The nucleic acid vector of any of paragraphs 12-21, wherein the portion of GSH nucleic acid is at least 1 kb in length.
24. The nucleic acid vector of any of paragraphs 12-22, wherein the portion of GSH nucleic acid is between 300-3 kb in length.
25. The nucleic acid vector of any of paragraphs 12-22, wherein the portion of the GSH is a target site for a guide RNA (gRNA).
26. The nucleic acid vector of paragraph 25, wherein the gRNA is for a sequence-specific nuclease selected from any of: a TAL-nuclease, a zinc-finger nuclease (ZFN), a meganuclease, a megaTAL, or an RNA guide endonuclease (e.g., CAS9, cpf1, nCAS9).
27. The nucleic acid vector of any of paragraphs 12-26, the nucleic acid vector is a non-viral vector selected from the group comprising: a plasmid, a minicircle, comsid, an artificial chromosome (e.g., BAC), a linear covalently closed (LCC) DNA vector (e.g., minicircles, minivectors and miniknots), a linear covalently closed (LCC) vector (e.g., MIDGE, MiLV, ministering, miniplasmids), a mini-intronic plasmid, a pDNA expression vector, or variants thereof
28. The nucleic acid vector of paragraph 12, wherein the viral vector is selected from any of the group comprising: rAd, rAAV, rHSV, poxvirus vectors, lentivirus, vaccinia virus vectors, HSV Type 1 (HSV-1)-AAV hybrid vectors, baclulovirus expression vector systems (BEVS), and variants thereof
29. The nucleic acid vector of any of paragraphs 12-27, wherein the vector composition is a minicircle.
30. The nucleic acid vector of any of paragraphs 12-28, wherein the vector composition is an AAV vector comprising a capsid protein.
31. A nucleic acid vector composition comprising, in the following order:
   a. a GSH 5′ homology arm,
   b. a nucleic acid sequence comprising a restriction cloning site,
   c. a GSH 3′ homology arm, and
   wherein the 5′ homology arm and the 3′ homology arm bind to a target site located in a genomic safe harbor (GSH) locus identified in the method of any of paragraphs 1 to 11, and wherein the 5′ and 3′ homology arms guide homologous recombination into a loci located within the genomic safe harbor.
32. The vector composition of paragraph 31, wherein the 5′ and 3′ homology arms are between 30-2000 bp in length.
33. The vector composition of paragraphs 31 or 32, further comprising, inserted at the restriction cloning site, at least one or more of the following:
   a gene editing nucleic acid sequence;
   a target site for one or more nucleases;
   a nucleic acid of interest; or
   a guide RNA (gRNA) for a RNA-guided DNA endonuclease.
34. The vector composition of paragraph 33, wherein the gene editing nucleic acid sequence encodes a gene editing nucleic acid molecule selected from the group consisting of: a sequence-specific nuclease, one or more guide RNA (gRNA), CRISPR/Cas, a ribonucleoprotein (RNP) or any combination thereof.
35. The vector composition of paragraph 34, wherein the sequence-specific nuclease comprises: a TAL-nuclease, a zinc-finger nuclease (ZFN), a meganuclease, a megaTAL, or an RNA guide endonuclease (e.g., CAS9, cpf1, nCAS9).
36. The vector composition of paragraph 33, wherein the nucleic acid of interest is a miRNA, RNAi, encodes a therapeutic protein, antibody, peptide, suicide gene, apoptosis gene or any gene or combination of genes listed in Table 3.
37. The vector composition of paragraph 31, further comprising a control element, promoter or regulatory element operatively linked to the nucleic acid of interest.
38. The vector composition of any of paragraphs 31-37, wherein nucleic acid of interest or gene editing nucleic acid sequence is in an orientation for integration in the GSH in a forward orientation.
39. The vector composition of any of paragraphs 31-38, wherein nucleic acid of interest or gene editing nucleic acid sequence is in an orientation for integration in the GSH in a reverse orientation.
40. The vector composition of any of paragraphs 31-39, wherein GSH 5′ homology arm and the GSH 3′ homology arm bind to target sites that are spatially distinct nucleic acid sequences in the genomic safe harbor identified in the method of any of paragraphs 1 to 11.
41. The vector composition of any of paragraphs 31-40, wherein the GSH 5′ homology arm and the GSH 3′ homology arm are at least 65% complementary to a target sequence in the genomic safe harbor locus identified in the method of any of paragraphs 1 to 11.
42. The vector composition of any of paragraphs 31-40, wherein the GSH 5′ homology arm and the 3′ homology arm bind to a target site located in the PAX5 genomic safe harbor sequence.
43. The vector composition of any of paragraphs 31-42, wherein the GSH 5′ homology arm and the GSH 3′ homology arm are at least 65% complementary to at least part the PAX5 genomic safe harbor sequence.
44. The vector composition of any of paragraphs 31-41, wherein the GSH 5′ homology arm and the GSH 3′ homology arm bind to a GSH of target site located in a gene selected from Table 1A or 1B.
45. The vector composition of any of paragraphs 31-44, wherein the nucleic acid vector is a non-viral vector selected from the group consisting of: a plasmid, a minicircle, comsid, an artificial chromosome (e.g., BAC), a linear covalently closed (LCC) DNA vector (e.g., minicircles, minivectors and miniknots), a linear covalently closed (LCC) vector (e.g., MIDGE, MiLV, ministering, miniplasmids), a mini-intronic plasmid, a pDNA expression vector, or variants thereof
46. The vector composition of any of paragraphs 31-44, wherein the nucleic acid is a viral vector selected from the group consisting of: rAd, rAAV, rHSV, poxvirus vectors, lentivirus, vaccinia virus vectors, HSV Type 1 (HSV-1)-AAV hybrid vectors, baclulovirus expression vector systems (BEVS) and variants thereof
47. The vector composition of any of paragraphs 31-44, wherein the vector composition is a minicircle.
48. The vector composition of any of paragraphs 31-44, wherein the vector composition is a AAV vector comprising a capsid protein.
49. A cell comprising the vector composition of any of paragraphs 12-48.
50. The cell of paragraph 49, wherein the cell is a red blood cell (RBC) or RBC precursor cell.
51. The cell of paragraph 50, wherein the RBC precursor cell is a CD44+ or CD34+ cell.
52. The cell of paragraph 49, wherein the cell is a stem cell.
53. The cell of paragraph 49, wherein the cell is an iPS cell or embryonic stem cell.
54. The cell of paragraph 54, wherein the iPS cell is a patient-derived iPSC.
55. The cell of any of paragraphs 49-54, wherein the cell is a mammalian cell.
56. The cell of paragraph 55, wherein the mammalian cell is a human cell.
57. A method for inserting a nucleic acid of interest or gene editing nucleic acid sequence into a genomic safe harbor (GSH) loci of a cell, the method comprising introducing the vector of any of paragraphs 31-48 into the cell, whereby homologous recombination of 3′ and 5′ homology arms with regions of the GSH integrate the nucleic acid sequence or gene editing nucleic acid sequence into the GSH loci.
58. The method of paragraph 57, wherein the nucleic acid sequence is integrated into the GSH in a forward orientation.
59. The method of paragraph 57, wherein the nucleic acid sequence is integrated into the GSH in a reverse orientation.
60. A cell comprising an integrated nucleic acid of interest or gene editing nucleic acid sequence located in a genomic safe harbor (GSH) loci selected from Table 1A or 1B.
61. The cell of paragraph 60, produced by the method of paragraph 56.
62. The cell of paragraphs 60 or 61, wherein the cell is a red blood cell (RBC) or RBC precursor cell.
63. The cell of paragraph 62, wherein the RBC precursor cell is a CD44+ or CD34+ cell.
64. The cell of any of paragraphs 60-61, wherein the cell is a stem cell.
65. The cell of any of paragraphs 60-61, wherein the cell is an iPS cell or embryonic stem cell.
66. The cell of any of paragraphs 60-65, wherein the iPS cell is a patient-derived iPSC.
67. The cell of any of paragraphs 60-66, wherein the cell is a mammalian cell.
68. The cell of paragraph 67, wherein the cell is a human cell.
69. A transgenic organism comprising an integrated nucleic acid of interest or gene editing nucleic acid sequence located in a genomic safe harbor loci selected from Table 1A or 1B.
70. The transgenic organism of paragraph 69, wherein the nucleic acid of interest or gene editing nucleic acid sequence is integrated into the GSH loci according to the method of paragraph 56.
71. A kit comprising:
   a. A vector composition of any of paragraphs 31-48; and
   b. at least one GSH 5′ primer and at least one GSH 3′ primer, wherein the GSH is identified by the method of any of paragraphs 1 to 11, wherein the at least one GSH 5′ primer binds to a region of the GSH upstream of the site of integration, and the at least one GSH 3′ primer is at least binds to a region of the GSH downstream of the site of integration; and/or
      i. at least two GSH 5′ primers comprising a forward GSH 5′ primer that binds to a region of the GSH upstream of the site of integration, and a reverse GSH 5′ primer that binds to a sequence in the nucleic acid inserted at the site of integration in the GSH sequence, wherein the GSH is identified by the method of any of paragraphs 1 to 11.
c. at least two GSH 3′ primers comprising a forward GSH 3′ primer that binds to a sequence located at the 3′ end of the nucleic acid inserted at the site of integration in the GSH sequence, and a reverse GSH 3′ primer binds to a region of the GSH downstream of the site of integration, and wherein the GSH is identified by the method of any of paragraphs 1 to 11.
72. A kit comprising: (a) a GSH-specific single guide and an RNA guided nucleic acid sequence comprised in one or more GSH vectors; and (b) GSH knock-in vector comprising GSH vector, wherein one or more of the sequences of (a) or (b) are comprised on a vector of any of paragraphs 31-48.
73. The kit of paragraph 72, wherein the GSH vector is a GSH-CRISPR-Cas vector.
74. The kit of paragraph 72, wherein the GSH CRISPR-Cas vector comprises a GSH-sgRNA nucleic acid sequence and Cas9 nucleic acid sequence.
75. The kit of paragraph 72, comprising a GSH knockin-donor vector comprising a GSH 5′ homology arm and a GSH 3′ homology arm, wherein the GSH 5′ homology arm and the GSH 3′ homology arm are at least 65% complementary to a sequence in the genomic safe harbor (GSH) identified in the method of any of paragraphs 1 to 11, and wherein the GSH 5′ and 3′ homology arms guide insertion by homologous recombination, of the nucleic acid sequence located between the GSH 5′ homology arm and a GSH 3′ homology arm into a loci located within the genomic safe harbor identified in the method of paragraph 1 or 11.
76. The kit of paragraph 72, wherein the GSH knockin-donor vector is a PAX5 knockin-donor vector comprising a PAX5 5′ homology arm and a PAX5 3′ homology arm, wherein the PAX5 5′ homology arm and the PAX5 3′ homology arm are at least 65% complementary to the PAX5 genomic safe harbor loci, and wherein the PAX5 5′ and 3′ homology arms guide insertion, by homologous recombination, of the nucleic acid located between the GSH 5′ homology arm and a GSH 3′ homology arm into a loci within the PAX5 genomic safe harbor.
77. The kit of paragraph 72, wherein the GSH knockin-donor vector is a knockin donor vector comprising a 5′ homology arm which binds to a GSH loci listed in Table 1A or 1B, and a 3′ homology arm which binds to a spatially distinct region of the same GSH loci that the 5′ homology arm binds to, wherein the 5′ and 3′ homology arms guide insertion, by homologous recombination, of the nucleic acid located between the GSH 5′ homology arm and a GSH 3′ homology arm into a GSH loci listed in Table 1A or 1B.
78. The kit of paragraph 72, wherein the GSH vector is GSH Cas9 knock in donor vector. The kit of any of paragraphs 72-78, further comprising at least one GSH 5′ primer and at least one GSH 3′ primer, wherein the GSH is identified by the method of any of paragraphs 1 to 11, wherein the at least one GSH 5′ primer is at least 80% complementary to a region of the GSH upstream of the site of integration, and the at least one GSH 3′ primer is at least 80% complementary to a region of the GSH downstream of the site of integration.
79. The kit of any of paragraphs 72-79, further comprising at least two GSH 5′ primers comprising;
   a. a forward GSH 5′ primer that is at least 80% complementary to a region of the GSH upstream of the site of integration, and
   b. a reverse GSH 5′ primer that is at least 80% complementary to a sequence in the nucleic acid inserted at the site of integration in the GSH sequence,
   wherein the GSH is identified by the method of any of paragraphs 1 to 11.
80. The kit of any of paragraphs 72-80, further comprising at least two GSH 3′ primers comprising;
   a. a forward GSH 3′ primer that is at least 80% complementary to a sequence located at the 3′ end of the nucleic acid inserted at the site of integration in the GSH sequence, and
   b. a reverse GSH 3′ primer that is at least 80% complementary to a region of the GSH downstream of the site of integration, and
   wherein the GSH is identified by the method of any of paragraphs 1 to 11.
81. The kit of any of paragraphs 72-81, wherein the GSH 5′ primer is a PAX5 5′ primer and the GSH 3′ primer is a PAX 3′ primer, wherein the PAX5 5′ primer and the PAX5 3′ primer flank the site of integration in the PAX5 genomic safe harbor.
82. A transgenic mouse comprising a marker gene inserted into the genomic DNA of the mouse at a GSH loci identified according to the methods of any of paragraphs 1 to 11, wherein the reporter gene is flanked by lox sites.
83. The transgenic mice of paragraph 83, wherein the lox sites are LoxP sites.
84. The transgenic mice of paragraph 83, wherein the GSH loci is located in the genomic DNA of any of the genes selected from Table 1A or 1B.
85. The transgenic mice of paragraph 83, wherein the GSH loci is located in the intronic or untranslated region (e.g., 3′UTR, 5′UTR exonic) nucleic acid sequence of the PAX5 gene or Kif1 gene.
86. A method of generating a genetically modified animal comprising a nucleic acid interest inserted at a Genomic Safe Harbor (GSH) loci identified according to the method of any of paragraphs 1 to 11, comprising a) introducing into a host cell a vector of any of paragraphs 24-42, and b) introducing the cell generated in (a) into a carrier animal to produce a genetically modified animal.
87. The method of paragraph 87, wherein the host cell is a zygote or a pluripotent stem cell.
88. A genetically modified animal produced by the method of paragraph 87.
89. A recombinant dependoparvovirus vector comprising a capsid, wherein the capsid comprises at least one GSH nucleic acid sequence.
90. The recombinant dependoparvovirus vector of paragraph 90, wherein the GSH nucleic acid sequence is identified by the method of any of paragraphs 1-11.
91. The recombinant dependoparvovirus vector of paragraph 90, wherein the GSH nucleic acid sequence is an EVE.
92. The recombinant dependoparvovirus vector of paragraph 91 or 92, wherein the capsid comprises sequence that is not found in the capsids of any of wild-type AAV I, II, III, IV, V, VI, VII, VIII or IX.
93. The recombinant dependoparvovirus vector of any of paragraphs 90-93, wherein the dependoparvovirus is an AAV.

### Definitions

The term “Genomic Safe Harbor” is also interchangeably referred to herein as “GSH” or “safe harbor gene” or “safe harbor locus” refers to a location within a genome, including a region of genomic DNA or a specific site, that can be used for integrating an exogenous nucleic acid wherein the integration does not cause any significant deleterious effect on the growth of the host cell by the addition of the exogenous nucleic acid alone. That is, a GSH refers to a gene or loci in the genome that a nucleic acid sequence can be inserted such that the sequence can integrate and function in a predictable manner (e.g., express a protein of interest) without significant negative consequences to endogenous gene activity, or the promotion of cancer. For example, a genomic safe harbor (GSHs) is a site in the host cells genome that is able to accommodate the integration of new genetic material in a manner that ensures that the newly inserted genetic elements (i) function predictably and (ii) do not cause significant alterations of the host genome thereby averting a risk to the host cell or organism, and (iii) preferably the inserted nucleic acid is not perturbed by any read-through expression from neighboring genes, and (iv), does not activate nearby genes. GSHs can be a specific site, or can be a region of the genomic DNA. A GSH can be a chromosomal site where transgenes can be stably and reliably expressed in all tissues of interest without adversely affecting endogenous gene structure or expression. In some embodiments, a safe harbor gene is also a loci or gene where an inserted nucleic acid sequence can be expressed efficiently and at higher levels than a non-safe harbor site.

The term “loci” is the plural of “locus” and refers to the position in a chromosome of a particular gene, target site of integration, or GSH.

The term “GSH loci” refers to a region of the chromosome of where integration does not cause any significant effect on the growth or differentiation of the target cell by the addition of the nucleic acid alone.

The term “endogenous viral element” or “EVE” is a DNA sequence derived from a virus, and present within the germline of a non-viral organism. EVEs may be entire viral genomes (proviruses), or fragments of viral genomes. They arise when a viral DNA sequence becomes integrated into the genome of a germ cell that goes on to produce a viable organism. The newly established EVE can be inherited from one generation to the next as an allele in the host species, and may even reach fixation.

The term “provirus” refers to the genome of a virus when it is integrated or inserted into a host cell's DNA. Provirus refers to the duplex DNA form of the retroviral genome linked to a cellular chromosome. The provirus is produced by reverse transcription of the RNA genome and subsequent integration into the chromosomal DNA of the host cell.

The term “parvovirus” refers to any species of the family (Parvoviridae) comprising or consisting of DNA viruses with linear single-stranded DNA genomes that include the causative agents of fifth disease in humans, panleukopenia in cats, and parvovirus infection in dogs and other carnivore host species.

The term “circovirus” is a genus of DNA viruses with a single-stranded circular genome (family Circoviridae), various species of which cause potentially lethal infections in swine, fowls, pigeons, and psittacine birds.

The term “proto-species” as disclosed herein refers to an ancestral species that gave rise to a group of related species or organisms consisting that may or may not be capable of exchanging genetic information and cross-breeding. The species is the principal natural taxonomic unit, ranking below a genus and denoted by a Latin binomial, e.g., Homo sapiens.

The term “orthologous” refers to genes in different species or organisms derived from a common ancestral gene following speciation from a common ancestral gene. Commonly, orthologues retain the same function in the course of evolution and are genes with similar sequence, however, as the host species evolved, the same gene may have been adapted to perform a different role. For example, piRNA (a crystalline gene of the eye) is a gene that is adapted to perform a different role, has it comprises a complex path of domain proteins. Orthologues in divergent species often have an identical function and in some embodiments, are often interchangeable between species without losing function, for example Metazomes in bacteria. Once a phylogenic tree used to establish phylogenetic relationships between species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the orthologue can be deduced from the identified function of the reference sequence. Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence).

The term “taxonomic order” refers to orderly classification of plants and animals according to their presumed natural relationships. Species relatedness, based on analysis of genomic sequence data provides a quantitative alternative approach to the natural relationships deduced from physical relationships.

The term “cetacea” refers to the taxonomic (infra)order of aquatic marine mammals comprising among others, baleen whales, toothed whales, dolphins and porpoises, and related forms and that have a torpedo-shaped nearly hairless body, paddle-shaped forelimbs but no hind limbs, one or two nares opening externally at the top of the head, and a horizontally flattened tail used for locomotion.

The term “chiroptera” refers to the taxonomic order of mammals capable of true flight, and comprise bats.

The term “lagomorpha” refers to the taxonomic order of gnawing herbivorous mammals having two pairs of incisors in the upper jaw one behind the other, usually soft fur, and short or rudimentary tail, made up of two families (Leporidae and Ochotonidae genera that comprise the Leporidae family) comprising the rabbits, hares, and pikas, and was formerly considered a suborder of the order Rodentia.

The term “Macropodidae” refers to the taxonomic family of diprotodont marsupial mammals comprising the kangaroos, wallabies, and rat kangaroos that are all saltatory animals with long hind limbs and weakly developed forelimbs and are typically inoffensive terrestrial herbivores.

The term “Rodentia” is of the taxonomic order of relatively small gnawing mammals (such as a mouse, squirrel, or beaver) that have in both jaws a single pair of incisors with a chisel-shaped edge. It includes all rodents.

The term “primates” is the taxonomic order of mammals that are characterized especially by advanced development of binocular vision resulting in stereoscopic depth perception, specialization of the hands and feet for grasping, and enlargement of the cerebral hemispheres and include humans, apes, monkeys, and related forms (such as lemurs and tarsiers).

The term “monotremata” refers to the taxonomic order of egg-laying mammals comprising the platypuses and echidnas.

The term “syntenic” refers to similar organization or ordering of a series of genes in different species.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hy-brids, or a polymer including purine and pyrimidine bases or other natural, chemically or biochemi-cally modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleo-tide. Oligonucleotides are also known as “oligomers” or “oligos” and may be isolated from genes, or chemically synthesized by methods known in the art. The terms “polynucleotide” and “nucleic ac-id” should be understood to include, as applicable to the embodiments being described, single-stranded (such as sense or antisense) and double-stranded polynucleotides.

By “nucleic acid of interest” is meant any nucleic acid sequence (including DNA and RNA sequences) which encodes a protein, RNA or other molecule which is desirable for delivery to a mammalian host cell. The sequence is generally operatively linked to other sequences which are needed for its expression such as a promoter. The phrase “nucleic acid of interest” is not meant to be limiting to DNA, but includes any nucleic acid (e.g., RNA or DNA) that encodes a protein or other molecule desirable for administration.

The term “nucleic acid construct” as used herein refers to a nucleic acid molecule, either single- or double-stranded, which is isolated from a naturally occurring gene or which is modified to con-tain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA]. In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the con-text of tRNA anti-codon base-pairing with codons in mRNA. In the context of this disclosure, a guanine (G) of a protein-binding segment (dsRNA duplex) of a subject DNA-targeting RNA mole-cule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is in-stead considered to be complementary.

The terms “peptide,” “polypeptide,” and “protein” are used interchangeably herein, and refer to a polymeric form of amino acids of any length, which can include coded and non-coded amino ac-ids, chemically or biochemically modified or derivatized amino acids, and polypeptides having modified peptide backbones.

A DNA sequence that “encodes” a particular RNA or protein gene product is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, a “promoter sequence” is a DNA regulatory region capable of binding RNA polymerase and initiating transcription of a downstream (3′ direction) coding or non-coding sequence. A promoter sequence may be bounded at its 3′ terminus by the transcription initiation site and ex-tends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present disclosure.

As used herein, the term “gene editing functionality” refers to the insertion, deletion or replacement of DNA at a specific site in the genome with a loss or gain of function. The insertion, deletion or replacement of DNA at a specific site can be accomplished e.g. by homology-directed repair (HDR) or non-homologous endjoining (NHEJ), or single base change editing. In some embodiments, a do-nor template is used, for example for HDR, such that a desired sequence within the donor template is inserted into the genome by a homologous recombination event. In one embodiment, a “donor template” or “repair template” comprises two homology arms (e.g., a 5′ homology arm and a 3′ homology arm) flanking on either side of a donor sequence comprising a desired mutation or insertion in the nucleic acid sequence to be introduced into the host genome. The 5′ and 3′ homology arms are substantially homologous to the genomic sequence of the target gene at the site of endo-nuclease mediated cutting. The 3′ homology arm is generally immediately downstream of the pro-tospacer adjacent motif (PAM) site where the endonuclease cuts (e.g., a double stranded DNA cut), or in some embodiments, nicks the DNA. The terms “DNA regulatory sequences,” “control elements,” and “regulatory elements,” used inter-changeably herein, refer to transcriptional and translational control sequences, such as promoters, enhancers, polyadenylation signals, terminators, protein degradation signals, and the like, that pro-vide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide. Typical “control elements” include, but are not limited to transcription promoters, transcription enhancer elements, cis-acting transcription regulating elements (transcription regulators, a cis-acting element that affects the transcription of a gene, for example, a region of a promoter with which a transcription factor interacts to modulate expression of a gene), transcription termination signals, as well as polyadenylation sequences (located 5′ to the translation stop codon), sequences for optimization of initiation of translation (located 5′ to the coding sequence), translation enhancing sequences, and translation termination sequences. Control elements are derived from any include functional fragments thereof, for example, polynucleotides between about 5 and about 50 nucleotides in length (or any integer therebetween); preferably between about 5 and about 25 nucleotides (or any integer therebetween), even more preferably between about 5 and about 10 nucleotides (or any integer therebetween), and most preferably 9-10 nucleotides. Transcription promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the promoter controls the level of transcription of the coding sequence in response to the presence or absence of one or more transcriptional regulatory factors on the promoter sequence. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

An “expression cassette” includes an exogenous DNA sequence that is operably linked to a promoter or other regulatory sequence sufficient to direct transcription of the transgene in the vector. Suitable promoters include, for example, tissue specific promoters. Promoters can also be of AAV origin. A vector expression cassette for use in the vectors described herein can include, for example, an expressible exogenous sequence (e.g., open reading frame) that encodes a protein that is either absent, inactive, or insufficient activity in the recipient subject or a gene that encodes a protein having a desired biological or a therapeutic effect. The exogenous sequence such as a donor sequence can encode a gene product that can function to correct the expression of a defective gene or transcript. The expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a marker protein (also referred to as a reporter protein) to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art. A “marker gene” or “reporter gene” or “reporter sequence” are used interchangeably herein, and refers to any sequence that produces a protein product that is easily measured, preferably in a routine assay. Suitable marker genes include, but are not limited to, Mel1, chloramphenicol acetyl transferase (CAT), light generating proteins such as GFP, luciferase and/or β-galactosidase. Suitable marker genes may also encode markers or enzymes that can be measured in vivo such as thymidine kinase, measured in vivo using PET scanning, or luciferase, measured in vivo via whole body luminometric imaging. Selectable markers can also be used instead of, or in addition to, reporters. Positive selection markers are those polynucleotides that encode a product that enables only cells that carry and express the gene to survive and/or grow under certain conditions. For example, cells that express neomycin resistance (Ned) gene are resistant to the compound G418, while cells that do not express Ned are skilled by G418. Other examples of positive selection markers including hygromycin resistance and the like will be known to those of skill in the art. Negative selection markers are those polynucleotides that encode a product that enables only cells that carry and express the gene to be killed under certain conditions. For example, cells that express thymidine kinase (e.g., herpes simplex virus thymidine kinase, HSV-TK) are killed when gancyclovir is added. Other negative selection markers are known to those skilled in the art. The selectable marker need not be a transgene and, additionally, reporters and selectable markers can be used in various combinations.

In principle, the expression cassette can include any gene that encodes a protein, polypeptide or RNA that is either reduced or absent due to a mutation or which conveys a therapeutic benefit when overexpressed is considered to be within the scope of the disclosure. The vector may comprise a template or donor nucleotide sequence used as a correcting DNA strand to be inserted after a double-strand break (or nick) provided by a nuclease. The vector may include a template nucleotide sequence used as a correcting DNA strand to be inserted after a double-strand break (or nick) provided by a guided RNA nuclease, meganuclease, or zinc finger nuclease. Preferably, non-inserted bacterial DNA is not present and preferably no bacterial DNA is present in the vector compositions provided herein. In some instances, the protein can change a codon without a nick.

Sequences provided in the expression cassette, expression construct, or donor sequence of a vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database.

Many organisms display a bias for use of particular codons to code for insertion of a particular amino acid in a growing peptide chain. Codon preference or codon bias, differences in codon us-age between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organ-ism based on codon optimization.

Given the large number of gene sequences available for a wide variety of animal, plant and microbial species, it is possible to calculate the relative frequencies of codon usage (Nakamura, Y., et al. “Codon usage tabulated from the international DNA sequence databases: status for the year 2000” Nucl. Acids Res. 28:292 (2000)).

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence.

As used herein, the term “host cell”, includes any cell type that is susceptible to transformation, transfection, transduction, and the like with a nucleic acid construct or vector of the present disclosure. As non-limiting examples, a host cell can be an isolated primary cell, pluripotent stem cells, CD34+ cells), induced pluripotent stem cells, or any of a number of immortalized cell lines (e.g., HepG2 cells). Alternatively, a host cell can be an in situ or in vivo cell in a tis-sue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a bio-logical system such as a cell or organism in which it is not normally found and one wishes to intro-duce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times. 100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues in the homology arm that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, Clus-talW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm of a repair template, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

As used herein, a “homology arm” refers to a polynucleotide that is suitable to target a donor sequence to a genome through homologous recombination. Typically, two homology arms flank the donor sequence, wherein each homology arm comprises genomic sequences upstream and down-stream of the loci of integration.

As used herein, “a donor sequence” refers to a polynucleotide that is to be inserted into, or used as a repair template for, a host cell genome. The donor sequence can comprise the modification which is desired to be made during gene editing. The sequence to be incorporated can be introduced into the target nucleic acid molecule via homology directed repair at the target sequence, thereby causing an alteration of the target sequence from the original target sequence to the sequence comprised by the donor sequence. Accordingly, the sequence comprised by the donor sequence can be, relative to the target sequence, an insertion, a deletion, an indel, a point mutation, a repair of a mutation, etc. The donor sequence can be, e.g., a single-stranded DNA molecule; a double-stranded DNA molecule; a DNA/RNA hybrid molecule; and a DNA/modRNA (modified RNA) hybrid molecule. In one embodiment, the donor sequence is foreign to the homology arms. The editing can be RNA as well as DNA editing. The donor sequence can be endogenous to or exogenous to the host cell genome, depending upon the nature of the desired gene editing.

“Heterologous,” as used herein, means a nucleotide or polypeptide sequence that is not found in the native nucleic acid or protein, respectively.

By “transformed cell” is meant a cell into which (or into an ancestor of which) has been introduced, by means of recombinant nucleic acid techniques, a nucleic acid molecule, i.e., a sequence of codons formed of nucleic acids (e.g., DNA or RNA) encoding a protein of interest. The introduced nucleic acid sequence may be present as an extrachromosomal or chromosomal element.

By “transformed cell” is meant a cell into which (or into an ancestor of which) has been introduced, by means of recombinant nucleic acid techniques, a nucleic acid molecule, i.e., a sequence of codons formed of nucleic acids (e.g., DNA or RNA) encoding a protein of interest. The introduced nucleic acid sequence may be present as an extrachromosomal or chromosomal element.

A “vector” or “expression vector” is a replicon, such as plasmid, bacmid, phage, virus, virion, or cosmid, to which another DNA segment, i.e. an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a plasmid or viral vector. The term “vector” encompasses any genetic element that is capable of replication when associated with the proper control elements and that can transfer gene sequences to cells. In some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, “Rep” refers to any AAV non-structural replicase or Rep protein or combination of AAV Rep proteins, e.g., Rep 78 and/or Rep 68 which is/are capable of providing the necessary function(s) to allow for replication of the viral genome, for example if an AAV ITR is used. In some embodiments, a different rolling circle replication protein is used (replicative protein sites), for example when the ITR is not an AAV ITR. Rep may also be used on non-AAV ITRs.

The terms “Correcting”, “genome editing” and “restoring” as used herein refers to changing a mutant gene that encodes a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

The phrase “Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually re-pairs the break accurately, yet imprecise repair leading to loss of nucleotides may also occur, but is much more common when the overhangs are not compatible “Nuclease mediated NHEJ” as used herein refers to NHEJ that is initiated after a nuclease, such as a cas9 or other nuclease, cuts double stranded DNA. In a CRISPR/CAS system NHEJ can be targeted by using a single guide RNA sequence.

“Homology-directed repair” or “HDR” as used interchangeably herein refers to a mechanism in cells to repair double strand DNA lesions when a homologous piece of DNA is present in the nucleus. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, non-homologous end joining may take place instead. In a CRISPR/Cas system one guide RNA, or two different guide RNAS can be used for HDR.

“Repeat variable diresidue” or “RVD” as used interchangeably herein refers to a pair of adjacent amino acid residues within a DNA recognition motif (also known as “RVD module”), which includes 33-35 amino acids, of a TALE DNA-binding domain. The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region.

“Site-specific nuclease” or “sequence specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems, that use various natural and unnatural Cas enzymes.

By “promoter” is meant a minimal DNA sequence sufficient to direct transcription. “Promoter” is also meant to encompass those promoter elements sufficient for promoter-dependent gene expression controllable for cell-type specific, tissue-specific or inducible by external signals or agents; such elements may be located in the 5′ or 3′ regions of the native gene.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.,” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Example 1: Identifying GSH

Herein, the inventors have discovered that all Cetacea have an intronic AAV EVE in the PAX5 gene. The inventors assessed if this EVE locus, e.g., the PAX5 gene is a safe-harbor by inserting a reporter gene into the orthologous region in human progenitor cells. Using mouse and human lymphomyeloid stem cells, the inventors will insert a marker gene into the PAX5 gene ex vivo and then engrafted the cells into immune-cell depleted mice. The lymphomyeloid cells differentiate and repopulate the lineages which are easily characterized with cell surface markers. The inventors are also to assess transgenic mice with a marker gene inserted into the PAX5 gene to test of the breadth of the safe-harbor.

### Example 2: Making DNA Vectors in General

An exemplary vector with a 5′ GSH-specific homology arm and a 3′ GSH-specific homology arm are made where the 5′ GSH-specific homology arm and a 3′ GSH-specific homology arm are specific to a GSH identified herein, e.g., Pax5 or a GSH identified in Table 1A or Table 1B. In such an experiment, Plasmids that comprise in this order: a 5′ GSH-specific homology arm, a nucleic acid of interest (e.g. a therapeutic nucleic acid), a 3′ GSH-specific homology arm. The plasmid may further comprise, a gene editing molecule, e.g. one or more of, at least one guide RNA directed to the GSH, and a nuclease (e.g., Cas9) CRISPR/Cas, ZFN or Tale nucleic acid sequences.

### Example 3: Vectors with a 5′- and 3′ GSH-Specific Homology Arms Express a Transgene or Nucleic Acid of Interest In Vivo

In vivo protein expression from vectors described above are determined in mice. 1002921A nucleic acid of interest-expressing open reading frame is inserted into the vector, flanked by 5′- and 3′ GSH-specific homology arms which bind to a GSH identified herein to facilitate HDR within the GSH loci. In some embodiments, the 5′- and 3′ GSH-specific homology arms are large (up to 2 Kb each). In experiments, the nucleic acid of interest in the vector is a nuclease expressing the open reading frame of a reporter protein, along with any needed adjunct components such as sgRNA, with the nuclease specific for a site at or near the GSH locus and effective to increase recombination. In some experiments, the vector is delivered in lipid nanoparticles (LNPs).

An exemplary test vector expression unit can be assessed in accordance with the present disclosure where the nucleic acid of interest is flanked by 5′ and 3′ GSH-specific homology arms complementary or substantially complementary to the GSH to allow for homologous recombination. In some embodiments, negative controls can be established, e.g., where a control vector can comprise scrambled homology arm sequences or no homology arms to check the efficiency of recombination may be more appropriate. In alternative embodiments, control vectors comprising only the 5′ GSH-specific homology arm; and/or a control vector containing only the 3′ GSH-specific homology arm, can be used to check for, and serve as a negative control for effective targeting by the other vector to target the GSH. An expression unit, such as a nucleic acid of interest can be a marker gene, (also referred to herein as a reporter gene), e.g., GFP, including a promoter, WPRE element, pA, can be used to experimentally confirm expression.

In some embodiments, validation of the GSH can be performed by assessing off-target sites, and/or using next generation sequencing with tag-specific sequences that amplify the GSH locus with an inserted transgene or reporter gene. Such analysis is useful for assessing specificity and/or efficiency of targeting a GSH locus with a vector with 3′- and 5-GSH specific homology arms.

A nuclease expressing unit can be delivered in trans, such Cas9 mRNA, zinc-finger nucleases (ZFN), transcription activator-like effector nucleases (TALEN), mutated “nickase” endonuclease, class II CRISPR/Cas system (CPF1). In experiments, LNPs can be used as a delivery option. The transport into the nuclei can be increased by using a nuclear localization signal (NLS) fused into the 5′ or 3′ enzyme peptide sequence, according to methods commonly known to persons of ordinary skill in the art. In another embodiment, the NLS can be inserted internally such that the NLS is exposed on the surface of the nuclease and does not interfere with its function as a nuclease.

Where appropriate for the nuclease, to induce double-stranded break (DSB) at the desired site one or more single guided RNA are delivered in trans as well; Either as an sgRNA expressing vector or chemically synthesized synthetic sgRNA. (sgRNA=single guide-RNA target sequence) as described herein. sgRNA can be selected using freely available software/algorithm, e.g., such as at tools.genome-engineering.org, can be used to select suitable single guide-RNA sequences.

The 5′ GSH-specific homology arm can be approximately 350 bp long, and can be in range between 50 to 2000 bp, as described herein. In some embodiments, the 3′ GSH-specific homology arm can be the same length or longer or shorter than the 5′ GSH-specific homology arm, and can be approximately 2000 bp long, or in the range of between 50 to 2000 bp, as described herein. Details study regarding length of homology arms and recombination frequency is e.g., reported by Jian-Ping Zhang et al., Genome Biology, 2017.

In further experiments, a therapeutic nucleic acid of interest ORF is substituted. In experiments, WPRE and polyadenylation signal, such as BGHpA can be added. In experiments, expression can also be regulated by the endogenous promoter of the GSH. In alternative embodiments, the promoter is a very strong promoter. In experiments, a translation enhancing element, such as WPRE is added 3′ of the ORF. In experiments, also, a polyadenylation signal (e.g., BGH-pA) is added needed as well.

In some embodiments, the GSH loci is PAX5 or any GSH listed in Table 1A or 1B. The hypothesis is the insert into an intron site without any effects on the target cell or tissue.

### Example 4

In some embodiments, expression constructs are made for titration of self-inactivating features of the nuclease activity by introducing sgRNA sequences in the intron of the synthetic promoter unit, e.g., the CAG promoter that regulates nuclease expression. The degree of inactivation is determined by the number of sgRNA seq or combination and/or mutated (de-optimized) sgRNA target seq. (Zhang et al, NatPro, 2013 Regulation of Cas9 activity by using de-optimized sgRNA recognition target sequence.)

In some embodiments, a vector is made containing a nuclease expression unit (including hashed nuclease element) and an intron downstream of the promoter having the illustrated sgRNA targeting sequence. The features can include, but are not limited to, Pol III promoter (U6 or H1) driven sgRNA expressing unit with optional orientation in regard the transcription direction; Synthetic promoter driven nuclease (e.g., Cas9, double mutant Nickase, Talen, or other mutants) expression unit that may contain sgRNA targeting sequences with or w/o de-optimization (in experiments, located other than as indicated); A nucleic acid of interest, (e.g, a transgene) potentially fused to a selection marker (e.g., NeoR) through a viral 2A peptide cleavage site (2A) flanked by 0.05 to 6 kb stretching homology arms. (On 2A systems: Chan et al, Comparison of IRES and F2A-Based Locus-Specific Multicistronic Expression in Stable Mouse LinesHSV-TK suicide, PLOS 2011 HSV-TK suicide gene system; Fesnak et al, Engineered T Cells: The Promise and Challenges of Cancer Immunotherapy, NatRevCan 2016.) If suitable, a selection marker (e.g., HSV TK) and expressing unit that allows to control and select for successful integration into the GSH can be positioned inside the 5′- and 3′ GSH-specific homology arms.

The 5′- and 3′ GSH-specific homology arms in the vector allow for an anticipated site of insertion by homologous recombination. However, if instead there is random integration, the entire vector with negative selectable marker is integrated into the genome. Such mis-transfected cells can be killed with appropriate drugs, such as GVC for the HSV TK negative selectable marker. In some embodiments, a negative selection marker can be replaced with a sgRNA target sequence for a “double mutant nickase” where the introduction of single stranded DNA cut (nicking) can help to release torsion downstream of the 3′ GSH-specific homology arm and increase annealing and therefore increase HDR frequency. In experiments, the negative marker is used with the sgRNA target sequence for “double mutant nickase.”

### Example 5: Transcriptomic Analysis

Safe harbor sites provide genomic loci for insertion of one or more transgenes of interest without disrupting other nearby loci. However, the ability to insert a gene at a locus safely does not necessarily indicate that that gene will be transcribed at a measurable or desired rate. Accordingly, studies were undertaken to examine transcription of an inserted marker at the identified genomic safe harbor sites kif6 and Pax as compared to transcription from the same marker gene at other insertion sites, including the known genomic safe harbor Adeno-Associated Virus integration Site 1 (AAV51), and two arbitrary control loci (DCTN and SRF), selected for their similar functionality type to Kif6 (structural protein) and Pax5 (regulatory protein). Briefly, HEK293 cells were engineered to have a green fluorescent protein (GFP) gene inserted at one of those loci, and by monitoring the presence of the GFP transcript the degree of expression of a gene inserted at that locus can be assessed.

Briefly, whole RNA sequencing (RNA-seq) was performed on cells having a GFP insertion at one of the loci of interest, using standard techniques. All paired end RNA-seq reads were initially assessed for quality with FASTQC (Andrews, 2010). Samples that passed through the quality threshold of 30 (Q>30) were aligned using the STAR Spliced Transcripts Alignment to a Reference) aligner software (Dobin et al., Bioinformatics 29(1): 15-21 (2013)) to the Ensembl human genome reference (GRCh38) and associated gene transfer format (GTF) file (GRCh38.94). Count data for each sample were generated from STAR-aligned BAM files using the internal flag in STAR. Multidimensional scaling (MDS) plots were generated using the Glimma software package (Su et al., Bioinforma, Oxf. Engl. 33: 2050-52 (2017)) in the R language using counts per million (CPM) data. Counts were made on a minimum of 3 samples to reflect all three replicates per cell line. Differential gene expression (DE) was identified with the software package EdgeR (McCarthy et al., Nucl. Acids Res. 40: 4288-97 (2012); Robinson et al., Bioinforma. Oxf. Engl. 26: 139-40 (2010)) using generalized linear models (GLMs) available through R/Bioconductor (R Core Team, 2016). Pairwise differences among means and linear combinations of model parameters were used to evaluate the DE between wildtype and the edited GSH cell lines with GFP integrated at the four candidate loci or the AAVs1 loci. Further analysis of the transcriptomes across different categories of expressed genes in the Kif6-inserted cells as well as the other cells further demonstrated no clustering in any one category of genes, indicating that especially in the case of Kif6, no categories of biological functions were particularly impaired by the insertion.

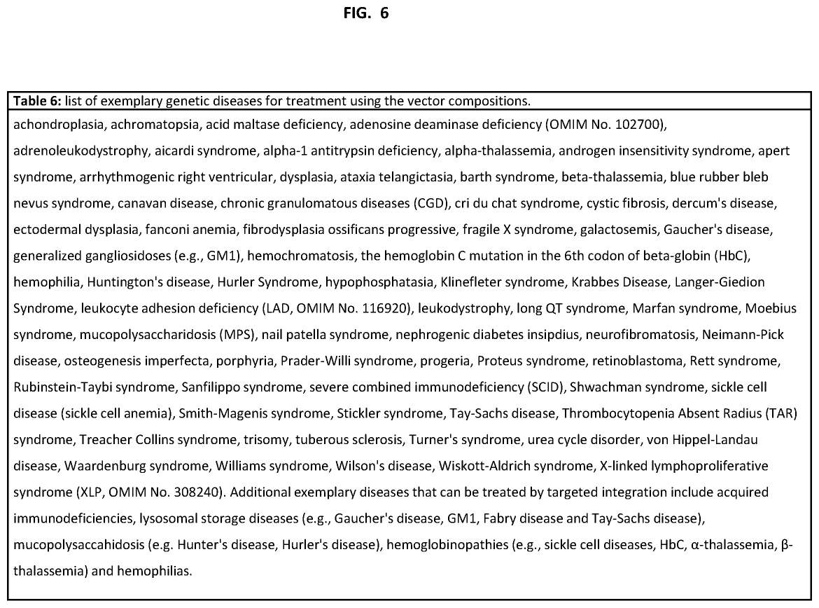

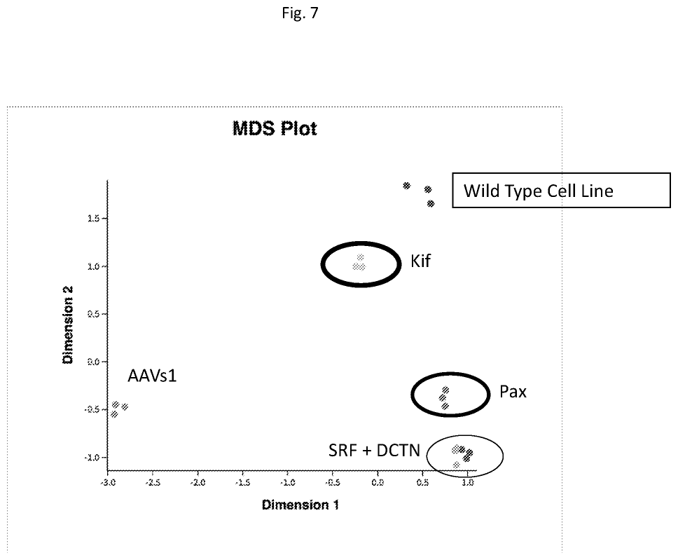

The results of the analysis are shown in FIG. 7. The transcriptomes from cells with the insertions at the arbitrary control loci DCTN or SRF demonstrated very similar profiles in the MDS plot (FIG. 7), but differed substantially from both AAV51 and wildtype cells. The cells with insertions at Kif6 and Pax5 were dissimilar to one another, with Pax5 near to the control samples and differing substantially from the AAVs 1-inserted cells, but Kif6 looking most similar to wild type cell transcriptomes. This suggested that insertion of a gene at the Kif6 locus had the least effect of any of the loci studied on the resulting cell expression profile and thus the least degree of cellular perturbation in response to the insertion at Kif6.

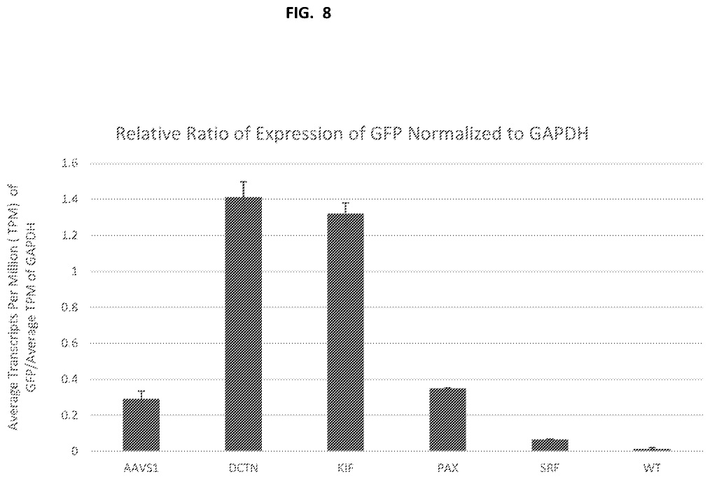

Next, the expression level of the GFP inserted at each of the loci was measured. To estimate GFP counts with respect to edited cell lines, the GTF file was amended to include GFP CDS and mapped back to the transcripts using the Salmon analysis tool (Patro et al., Nat. Methods 14: 417-419 (2017) and GAPDH as a comparator. The resulting transcripts per million (TPM) normalized data were collated and suitable comparisons charted to determine expression of the GFP transgene from integration at multiple loci. The results are shown in FIG. 8. Both the AAV51- and the Pax5-inserted cells displayed a moderate expression of GFP. The SRF-inserted cells had minimal GFP expression. Both DCTN and Kif6 had high levels of GFP expression (FIG. 8). These data suggested that both the Pax5 locus and the Kif6 locus are suitable safe harbor sites and can facilitate expression of genes inserted there, and Kif6 locus in particular has a near wild-type transcriptome and excellent expression of genes inserted there.

### REFERENCES

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.
Weitzman, et al., (2011). “Adeno-Associated Virus Biology”. In Snyder, R. O.; Moullier, P. Adeno-associated virus methods and protocols. Totowa, N.J.: Humana Press. ISBN 978-1-61779-370-7;
Mori S, et al., (2004). “Two novel adeno-associated viruses from cynomolgus monkey: pseudotyping characterization of capsid protein”. Virology. 330 (2): 375-83).
Chiorini, J. A., S. M. Wiener, R. A. Owens, S. R. Kyostio, R. M. Kotin, and B. Safer. 1994. ‘Sequence requirements for stable binding and function of Rep68 on the adeno-associated virus type 2 inverted terminal repeats’, J Virol, 68: 7448-57.
Chiorini, J. A., L. Yang, B. Safer, and R. M. Kotin. 1995. ‘Determination of adeno-associated virus Rep68 and Rep78 binding sites by random sequence oligonucleotide selection’, J Virol, 69: 7334-8.
DeKelver, et al., 2010. ‘Functional genomics, proteomics, and regulatory DNA analysis in isogenic settings using zinc finger nuclease-driven transgenesis into a safe harbor locus in the human genome’, Genome Res, 20: 1133-42.
Im, D. S., and N. Muzyczka. 1989. ‘Factors that bind to adeno-associated virus terminal repeats’, J Virol, 63: 3095-104.
Im, Dong-Soo, and Nicholas Muzyczka. “The AAV origin binding protein Rep68 is an ATP-dependent site-specific endonuclease with DNA helicase activity.” Cell 61.3 (1990): 447-457.
Im, D. S., and N. Muzyczka. “Partial purification of adeno-associated virus Rep78, Rep52, and Rep40 and their biochemical characterization.” Journal of virology 66.2 (1992): 1119-1128.
Kotin, R. M., and K. I. Berns. 1989. ‘Organization of adeno-associated virus DNA in latently infected Detroit 6 cells’, Virology, 170: 460-7.
Kotin, R. M., R. M. Linden, and K. I. Berns. 1992. ‘Characterization of a preferred site on human chromosome 19q for integration of adeno-associated virus DNA by non-homologous recombination’, EMBO J, 11: 5071-8.
Kotin, R. M., J. C. Menninger, D. C. Ward, and K. I. Berns. 1991. ‘Mapping and direct visualization of a region-specific viral DNA integration site on chromosome 19q13-qter’, Genomics, 10: 831-4.
Kotin, R. M., M. Siniscalco, R. J. Samulski, X. D. Zhu, L. Hunter, C. A. Laughlin, S. McLaughlin, N. Muzyczka, M. Rocchi, and K. I. Berns. 1990. ‘Site-specific integration by adeno-associated virus’, Proc Natl Acad Sci USA, 87: 2211-5.
Urcelay, E., P. Ward, S. M. Wiener, B. Safer, and R. M. Kotin. 1995. ‘Asymmetric replication in vitro from a human sequence element is dependent on adeno-associated virus Rep protein’, J Virol, 69: 2038-46.
Wang, J., G. Friedman, Y. Doyon, N. S. Wang, C. J. Li, J. C. Miller, K. L. Hua, J. J. Yan, J. E. Babiarz, P. D. Gregory, and M. C. Holmes. 2012. ‘Targeted gene addition to a predetermined site in the human genome using a ZFN-based nicking enzyme’, Genome Res, 22: 1316-26.
Weitzman, M. D., S. R. Kyostio, R. M. Kotin, and R. A. Owens. 1994. ‘Adeno-associated virus (AAV) Rep proteins mediate complex formation between AAV DNA and its integration site in human DNA’, Proc Natl Acad Sci USA, 91: 5808-12.
Zou, J., C. L. Sweeney, B. K. Chou, U. Choi, J. Pan, H. Wang, S. N. Dowey, L. Cheng, and H. L. Malech. 2011. ‘Oxidase-deficient neutrophils from X-linked chronic granulomatous disease iPS cells: functional correction by zinc finger nuclease-mediated safe harbor targeting’, Blood, 117: 5561-72.

1. A method to identify genomic safe harbor (GSH) regions in a mammalian genome, comprising;
a. identifying the loci of the endogenous virus element (EVE) of the genome of ur-species or in related species within taxonomic rank order;
b. identifying the interspecific conserved loci in the human or mouse genome;
c. validating the loci as a genomic safe harbors in human or mouse germlines using at least one in vitro or in vivo assays selected from any one or more of:
i. insertion of a marker gene into the loci in human cells and measure marker gene expression in vitro;
ii. insertion of marker gene into orthologous loci in progenitor cells or stem cells and engraft the cells into immune-depleted mice and/or assess marker gene expression in all developmental lineages;
iii. differentiate hematopoietic CD34+ cells into terminally differentiated cell types, wherein the hematopoietic CD34+ cells have a marker gene inserted into the loci identified in step b; or
iv. generate transgenic knock-in mouse wherein the genomic DNA of the mouse has a marker gene inserted in the loci identified in step b, wherein the marker gene is operatively linked to a tissue specific or inducible promoter.

2. The method of claim 1, wherein the GSH is intragenic or intergenic.

3. The method of claim 1, wherein the EVE is a nucleic acid sequence encoding intronic or exonic viral nucleic acid, viral DNA or DNA copies of viral RNA.

4. The method of claim 3, wherein the viral nucleic acid is non-retroviral nucleic acid or non-retroviral provirus.

5. The method of claim 4, wherein the non-retroviral nucleic acid is from a parvovirus or circovirus.

6. The method of claim 5, wherein the parvovirus is selected from group consisting of B19, minute virus of mice (mvm), RA-1, AAV, bufavirus, hokovirus, bocovirus, or any of the parvoviruses listing in Table 2 or Table 4A or Table 4B.

7. The method of claim 6, wherein the parvovirus is AAV.

8. The method of claim 5, wherein the circovirus is porcrine circovirus (PCV) (e.g., PCV-1, PCV-2).

9. The method of claim 4, wherein the non-retroviral nucleic acid encodes non-structural and/or structural viral proteins, e.g., rep (replication) and/or cap (capsid) proteins.

10. The method of claim 1, wherein the ur-species are selected from any of the group of: Cetacea, Chiropetera, Lagomorpha, Macropodiadae.

11. A method to identify genomic safe harbor (GSH) regions in a mammalian genome, comprising;
a) performing comparative genomic approaches to:
i) compare the interspecific introns of collinearly organized and/or synteny organized genes between species to identify an enlarged intron in one species relative to another species, and/or
ii) compare intergenic distance (or space) between adjacent genes or selected genes that are collinearly organized or synteny organized between species to identify a large variation in the intergenic distance (or space);
b) selecting the enlarged intron in step a(i) or intergenic space between selected genes in step a(ii) as a loci for a genomic safe harbor;
c) validating the loci as a genomic safe harbor in human or mouse germlines using at least one in vitro or in vivo assays selected from any one or more of:
i. insertion of a marker gene into the loci in human cells and measure marker gene expression in vitro;
ii. insertion of marker gene into orthologous loci in progenitor cells or stem cells and engraft the cells into immune-depleted mice and/or assess marker gene expression in all developmental lineages;
iii. differentiate hematopoietic CD34+ cells into terminally differentiated cell types, wherein the hematopoietic CD34+ cells have a marker gene inserted into the loci identified in step b; or
iv. generate transgenic knock-in mouse wherein the genomic DNA of the mouse has a marker gene inserted in the loci identified in step b, wherein the marker gene is operatively linked to a tissue specific or inducible promoter.

12. A nucleic acid vector comprising at least a portion of the genomic safe harbor (GSH) nucleic acid identified as a genomic safe harbor in the method of any of claims 1 to 11.

13. The nucleic acid vector of claim 12, wherein the vector is a viral vector or a non-viral vector.

14. The nucleic acid of claim 12, wherein the at least a portion of the GSH nucleic acid comprises the PAX5 genomic DNA or a fragment thereof.

15. The nucleic acid vector of claim 12, wherein the GSH nucleic acid comprises an untranslated sequence or an intron of the PAX5 gene.

16. The nucleic acid of claim 12, wherein the at least a portion of the GSH nucleic acid comprises the Kif5 genomic DNA or a fragment thereof.

17. The nucleic acid vector of claim 12, wherein the GSH nucleic acid comprises an untranslated sequence or an intron of the Kif5 gene.

18. The nucleic acid vector of claim 12, wherein the GSH nucleic acid is a nucleic acid selected from any of the nucleic acid sequences listed in Table 1A or Table 1B.

19. The nucleic acid vector of claim 12, wherein the at least portion of the GSH comprises at least one modification as compared to the wild-type GSH sequence.

20. The nucleic acid vector of claim 19, wherein the modification is a nucleic acid sequence comprising a restriction cloning site.

21. The nucleic acid vector of claim 19, wherein the modification is a nucleic acid sequence comprising one or more target sites for one or more nucleases.

22. The nucleic acid vector of claim 21, wherein the nuclease is selected from a zinc finger nuclease (ZFN), a TAL-effector domain nuclease (TALEN), or a CRISPR/Cas system.

23. The nucleic acid vector of any of claims 12-21, wherein the portion of GSH nucleic acid is at least 1 kb in length.

24. The nucleic acid vector of any of claims 12-22, wherein the portion of GSH nucleic acid is between 300-3 kb in length.

25. The nucleic acid vector of any of claims 12-22, wherein the portion of the GSH is a target site for a guide RNA (gRNA).

26. The nucleic acid vector of claim 25, wherein the gRNA is for a sequence-specific nuclease selected from any of: a TAL-nuclease, a zinc-finger nuclease (ZFN), a meganuclease, a megaTAL, or an RNA guide endonuclease (e.g., CAS9, cpf1, nCAS9).

27. The nucleic acid vector of any of claims 12-26, the nucleic acid vector is a non-viral vector selected from the group comprising: a plasmid, a minicircle, comsid, an artificial chromosome (e.g., BAC), a linear covalently closed (LCC) DNA vector (e.g., minicircles, minivectors and miniknots), a linear covalently closed (LCC) vector (e.g., MIDGE, MiLV, ministering, miniplasmids), a mini-intronic plasmid, a pDNA expression vector, or variants thereof.

28. The nucleic acid vector of claim 12, wherein the viral vector is selected from any of the group comprising: rAd, rAAV, rHSV, poxvirus vectors, lentivirus, vaccinia virus vectors, HSV Type 1 (HSV-1)-AAV hybrid vectors, baclulovirus expression vector systems (BEVS), and variants thereof.

29. The nucleic acid vector of any of claims 12-27, wherein the vector composition is a minicircle.

30. The nucleic acid vector of any of claims 12-28, wherein the vector composition is an AAV vector comprising a capsid protein.

31. A nucleic acid vector composition comprising, in the following order:
a. a GSH 5′ homology arm,
b. a nucleic acid sequence comprising a restriction cloning site,
c. a GSH 3′ homology arm, and
wherein the 5′ homology arm and the 3′ homology arm bind to a target site located in a genomic safe harbor (GSH) locus identified in the method of any of claims 1 to 11, and wherein the 5′ and 3′ homology arms guide homologous recombination into a loci located within the genomic safe harbor.

32. The vector composition of claim 31, wherein the 5′ and 3′ homology arms are between 30-2000 bp in length.

33. The vector composition of claim 31 or 32, further comprising, inserted at the restriction cloning site, at least one or more of the following:
a gene editing nucleic acid sequence;
a target site for one or more nucleases;
a nucleic acid of interest; or
a guide RNA (gRNA) for a RNA-guided DNA endonuclease.

34. The vector composition of claim 33, wherein the gene editing nucleic acid sequence encodes a gene editing nucleic acid molecule selected from the group consisting of: a sequence-specific nuclease, one or more guide RNA (gRNA), CRISPR/Cas, a ribonucleoprotein (RNP) or any combination thereof.

35. The vector composition of claim 34, wherein the sequence-specific nuclease comprises: a TAL-nuclease, a zinc-finger nuclease (ZFN), a meganuclease, a megaTAL, or an RNA guide endonuclease (e.g., CAS9, cpf1, nCAS9).

36. The vector composition of claim 33, wherein the nucleic acid of interest is a miRNA, RNAi, encodes a therapeutic protein, antibody, peptide, suicide gene, apoptosis gene or any gene or combination of genes listed in Table 3.

37. The vector composition of claim 31, further comprising a control element, promoter or regulatory element operatively linked to the nucleic acid of interest.

38. The vector composition of any of claims 31-37, wherein nucleic acid of interest or gene editing nucleic acid sequence is in an orientation for integration in the GSH in a forward orientation.

39. The vector composition of any of claims 31-38, wherein nucleic acid of interest or gene editing nucleic acid sequence is in an orientation for integration in the GSH in a reverse orientation.

40. The vector composition of any of claims 31-39, wherein GSH 5′ homology arm and the GSH 3′ homology arm bind to target sites that are spatially distinct nucleic acid sequences in the genomic safe harbor identified in the method of any of claims 1 to 11.

41. The vector composition of any of claims 31-40, wherein the GSH 5′ homology arm and the GSH 3′ homology arm are at least 65% complementary to a target sequence in the genomic safe harbor locus identified in the method of any of claims 1 to 11.

42. The vector composition of any of claims 31-40, wherein the GSH 5′ homology arm and the 3′ homology arm bind to a target site located in the PAX5 genomic safe harbor sequence.

43. The vector composition of any of claims 31-42, wherein the GSH 5′ homology arm and the GSH 3′ homology arm are at least 65% complementary to at least part the PAX5 genomic safe harbor sequence.

44. The vector composition of any of claims 31-41, wherein the GSH 5′ homology arm and the GSH 3′ homology arm bind to a GSH of target site located in a gene selected from Table 1A or 1B.

45. The vector composition of any of claims 31-44, wherein the nucleic acid vector is a non-viral vector selected from the group consisting of: a plasmid, a minicircle, comsid, an artificial chromosome (e.g., BAC), a linear covalently closed (LCC) DNA vector (e.g., minicircles, minivectors and miniknots), a linear covalently closed (LCC) vector (e.g., MIDGE, MiLV, ministering, miniplasmids), a mini-intronic plasmid, a pDNA expression vector, or variants thereof.

46. The vector composition of any of claims 31-44, wherein the nucleic acid is a viral vector selected from the group consisting of: rAd, rAAV, rHSV, poxvirus vectors, lentivirus, vaccinia virus vectors, HSV Type 1 (HSV-1)-AAV hybrid vectors, baclulovirus expression vector systems (BEVS) and variants thereof.

47. The vector composition of any of claims 31-44, wherein the vector composition is a minicircle.

48. The vector composition of any of claims 31-44, wherein the vector composition is a AAV vector comprising a capsid protein.

49. A cell comprising the vector composition of any of claims 12-48.

50. The cell of claim 49, wherein the cell is a red blood cell (RBC) or RBC precursor cell.

51. The cell of claim 50, wherein the RBC precursor cell is a CD44+ or CD34+ cell.

52. The cell of claim 49, wherein the cell is a stem cell.

53. The cell of claim 49, wherein the cell is an iPS cell or embryonic stem cell.

54. The cell of claim 54, wherein the iPS cell is a patient-derived iPSC.

55. The cell of any of claims 49-54, wherein the cell is a mammalian cell.

56. The cell of claim 55, wherein the mammalian cell is a human cell.

57. A method for inserting a nucleic acid of interest or gene editing nucleic acid sequence into a genomic safe harbor (GSH) loci of a cell, the method comprising introducing the vector of any of claims 31-48 into the cell, whereby homologous recombination of 3′ and 5′ homology arms with regions of the GSH integrate the nucleic acid sequence or gene editing nucleic acid sequence into the GSH loci.

58. The method of claim 57, wherein the nucleic acid sequence is integrated into the GSH in a forward orientation.

59. The method of claim 57, wherein the nucleic acid sequence is integrated into the GSH in a reverse orientation.

60. A cell comprising an integrated nucleic acid of interest or gene editing nucleic acid sequence located in a genomic safe harbor (GSH) loci selected from Table 1A or 1B.

61. The cell of claim 60, produced by the method of claim 56.

62. The cell of claim 60 or 61, wherein the cell is a red blood cell (RBC) or RBC precursor cell.

63. The cell of claim 62, wherein the RBC precursor cell is a CD44+ or CD34+ cell.

64. The cell of any of claims 60-61, wherein the cell is a stem cell.

65. The cell of any of claims 60-61, wherein the cell is an iPS cell or embryonic stem cell.

66. The cell of any of claims 60-65, wherein the iPS cell is a patient-derived iPSC.

67. The cell of any of claims 60-66, wherein the cell is a mammalian cell.

68. The cell of claim 67, wherein the cell is a human cell.

69. A transgenic organism comprising an integrated nucleic acid of interest or gene editing nucleic acid sequence located in a genomic safe harbor loci selected from Table 1A or 1B.

70. The transgenic organism of claim 69, wherein the nucleic acid of interest or gene editing nucleic acid sequence is integrated into the GSH loci according to the method of claim 56.

71. A kit comprising:
a. A vector composition of any of claims 31-48; and
b. at least one GSH 5′ primer and at least one GSH 3′ primer, wherein the GSH is identified by the method of any of claims 1 to 11, wherein the at least one GSH 5′ primer binds to a region of the GSH upstream of the site of integration, and the at least one GSH 3′ primer is at least binds to a region of the GSH downstream of the site of integration; and/or
i. at least two GSH 5′ primers comprising a forward GSH 5′ primer that binds to a region of the GSH upstream of the site of integration, and a reverse GSH 5′ primer that binds to a sequence in the nucleic acid inserted at the site of integration in the GSH sequence, wherein the GSH is identified by the method of any of claims 1 to 11.
c. at least two GSH 3′ primers comprising a forward GSH 3′ primer that binds to a sequence located at the 3′ end of the nucleic acid inserted at the site of integration in the GSH sequence, and a reverse GSH 3′ primer binds to a region of the GSH downstream of the site of integration, and wherein the GSH is identified by the method of any of claims 1 to 11.

72. A kit comprising:
(a) a GSH-specific single guide and an RNA guided nucleic acid sequence comprised in one or more GSH vectors; and
(b) GSH knock-in vector comprising GSH vector,
wherein one or more of the sequences of (a) or (b) are comprised on a vector of any of claims 31-48.

73. The kit of claim 72, wherein the GSH vector is a GSH-CRISPR-Cas vector.

74. The kit of claim 72, wherein the GSH CRISPR-Cas vector comprises a GSH-sgRNA nucleic acid sequence and Cas9 nucleic acid sequence.

75. The kit of claim 72, comprising a GSH knockin-donor vector comprising a GSH 5′ homology arm and a GSH 3′ homology arm, wherein the GSH 5′ homology arm and the GSH 3′ homology arm are at least 65% complementary to a sequence in the genomic safe harbor (GSH) identified in the method of any of claims 1 to 11, and wherein the GSH 5′ and 3′ homology arms guide insertion by homologous recombination, of the nucleic acid sequence located between the GSH 5′ homology arm and a GSH 3′ homology arm into a loci located within the genomic safe harbor identified in the method of claim 1 or 11.

76. The kit of claim 72, wherein the GSH knockin-donor vector is a PAX5 knockin-donor vector comprising a PAX5 5′ homology arm and a PAX5 3′ homology arm, wherein the PAX5 5′ homology arm and the PAX5 3′ homology arm are at least 65% complementary to the PAX5 genomic safe harbor loci, and wherein the PAX5 5′ and 3′ homology arms guide insertion, by homologous recombination, of the nucleic acid located between the GSH 5′ homology arm and a GSH 3′ homology arm into a loci within the PAX5 genomic safe harbor.

77. The kit of claim 72, wherein the GSH knockin-donor vector is a knockin donor vector comprising a 5′ homology arm which binds to a GSH loci listed in Table 1A or 1B, and a 3′ homology arm which binds to a spatially distinct region of the same GSH loci that the 5′ homology arm binds to, wherein the 5′ and 3′ homology arms guide insertion, by homologous recombination, of the nucleic acid located between the GSH 5′ homology arm and a GSH 3′ homology arm into a GSH loci listed in Table 1A or 1B.

78. The kit of claim 72, wherein the GSH vector is GSH Cas9 knock in donor vector.

79. The kit of any of claims 72-78, further comprising at least one GSH 5′ primer and at least one GSH 3′ primer, wherein the GSH is identified by the method of any of claims 1 to 11, wherein the at least one GSH 5′ primer is at least 80% complementary to a region of the GSH upstream of the site of integration, and the at least one GSH 3′ primer is at least 80% complementary to a region of the GSH downstream of the site of integration.

80. The kit of any of claims 72-79, further comprising at least two GSH 5′ primers comprising;
a. a forward GSH 5′ primer that is at least 80% complementary to a region of the GSH upstream of the site of integration, and
b. a reverse GSH 5′ primer that is at least 80% complementary to a sequence in the nucleic acid inserted at the site of integration in the GSH sequence,
wherein the GSH is identified by the method of any of claims 1 to 11.

81. The kit of any of claims 72-80, further comprising at least two GSH 3′ primers comprising;
a. a forward GSH 3′ primer that is at least 80% complementary to a sequence located at the 3′ end of the nucleic acid inserted at the site of integration in the GSH sequence, and
b. a reverse GSH 3′ primer that is at least 80% complementary to a region of the GSH downstream of the site of integration, and
wherein the GSH is identified by the method of any of claims 1 to 11.

82. The kit of any of claims 72-81, wherein the GSH 5′ primer is a PAX5 5′ primer and the GSH 3′ primer is a PAX 3′ primer, wherein the PAX5 5′ primer and the PAX5 3′ primer flank the site of integration in the PAX5 genomic safe harbor.

83. A transgenic mouse comprising a marker gene inserted into the genomic DNA of the mouse at a GSH loci identified according to the methods of any of claims 1 to 11, wherein the reporter gene is flanked by lox sites.

84. The transgenic mice of claim 83, wherein the lox sites are LoxP sites.

85. The transgenic mice of claim 83, wherein the GSH loci is located in the genomic DNA of any of the genes selected from Table 1A or 1B.

86. The transgenic mice of claim 83, wherein the GSH loci is located in the intronic or untranslated region (e.g., 3′UTR, 5′UTR exonic) nucleic acid sequence of the PAX5 gene or Kif1 gene.

87. A method of generating a genetically modified animal comprising a nucleic acid interest inserted at a Genomic Safe Harbor (GSH) loci identified according to the method of any of claims 1 to 11, comprising a) introducing into a host cell a vector of any of claims 24-42, and b) introducing the cell generated in (a) into a carrier animal to produce a genetically modified animal.

88. The method of claim 87, wherein the host cell is a zygote or a pluripotent stem cell.

89. A genetically modified animal produced by the method of claim 87.

90. A recombinant dependoparvovirus vector comprising a capsid, wherein the capsid comprises at least one GSH nucleic acid sequence.

91. The recombinant dependoparvovirus vector of claim 90, wherein the GSH nucleic acid sequence is identified by the method of any of claims 1-11.

92. The recombinant dependoparvovirus vector of claim 90, wherein the GSH nucleic acid sequence is an EVE.

93. The recombinant dependoparvovirus vector of claim 91 or 92, wherein the capsid comprises sequence that is not found in the capsids of any of wild-type AAV I, II, III, IV, V, VI, VII, VIII or IX.

94. The recombinant dependoparvovirus vector of any of claims 90-93, wherein the dependoparvovirus is an AAV.
